SEQUENCE LISTING (286 sequences)

SEQ ID NO: 1 - protein, 30 aa, synthetic construct
  VARIANT 1: note = X can be D, S or A
  VARIANT 4: note = X can be I or V
  VARIANT 5: note = X can be V, T, M, or A
  VARIANT 6: note = X can be D, N, or T
  VARIANT 7: note = X can be L or V
  VARIANT 8: note = X can be D or E
  VARIANT 10: note = X can be A or P
  VARIANT 11: note = X can be V, I, or G
  VARIANT 12: note = X can be D, E, or S
  VARIANT 13: note = X can be E or D
  VARIANT 14: note = X can be E, L, V, or absent
  VARIANT 16: note = X can be A or V
  VARIANT 17: note = X can be A, E, or K
  VARIANT 18: note = X can be L, V, or A
  VARIANT 19: note = X can be S, L, or V
  VARIANT 20: note = X can be V, I, G, T, or A
  VARIANT 21: note = X can be G or M
XLDXXXXXLXXXXXLXXXXXXGLGNTEVGA

SEQ ID NO: 2 - protein, 30 aa, synthetic construct
  VARIANT 6: note = X can be D, N, or T
  VARIANT 10: note = X can be A or P
  VARIANT 11: note = X can be V, I, or G
  VARIANT 18: note = X can be L, V, or A
DLDIVXLDLXXDEELAAXSVGGLGNTEVGA

SEQ ID NO: 3 - protein, 30 aa, synthetic construct
DLDIVDLDLAVDEELAALSVGGLGNTEVGA

SEQ ID NO: 4 - protein, 30 aa, synthetic construct
DLDIVNLDLPIDEELAAVSVGGLGNTEVGA

SEQ ID NO: 5 - protein, 30 aa, synthetic construct
DLDIVDLDLPIDEELAAVSIGGLGNTEVGA

SEQ ID NO: 6 - protein, 29 aa, synthetic construct
SLDVTTVELPGEDLVEALGMGLGNTEVGA

SEQ ID NO: 7 - protein, 29 aa, synthetic construct
SLDVMTVELPGEDLVKALGMGLGNTEVGA

SEQ ID NO: 8 - protein, 30 aa, synthetic construct
SLDVATVELPGSDLLVEAVTMGLGNTEVGA

SEQ ID NO: 9 - protein, 30 aa, synthetic construct
ALDVATVELPGSEVLVEAVAMGLGNTEIGA

SEQ ID NO: 10 - skipped

SEQ ID NO: 11 - protein, 7 aa, synthetic construct
  VARIANT 6: note = X can be V or L
  VARIANT 5: note = X can be Y, W, F, or H
  VARIANT 3: note = X can be any amino acid
  REGION >3: note = X can be repeated three to hundred times and wherein the last of the three to hundred amino acids is a positively charged amino acid
SGXSXXI

SEQ ID NO: 12 - protein, 9 aa, synthetic construct
  VARIANT 5: note = X can be any amino acid
  REGION >5: note = X can be repeated three to hundred times
  VARIANT 8: note = X can be V or L
SGFFXSWXI

SEQ ID NO: 13 - protein, 15 aa, synthetic construct
GGGGSGGGGSGGGGS

SEQ ID NO: 14 - protein, 8 aa, synthetic construct
GGGGGGGG

SEQ ID NO: 15 - protein, 6 aa, synthetic construct
GGGGGG

SEQ ID NO: 16 - protein, 15 aa, synthetic construct
EAAAKEAAAKEAAAK

SEQ ID NO: 17 - protein, 5 aa, synthetic construct
  REGION >5: note = The entire sequence of amino acids 1-5 can be repeated one to three times
EAAAK

SEQ ID NO: 18 - protein, 46 aa, synthetic construct
AEAAAKEAAAKEAAAKEAAAKALEAEAAAKEAAAKEAAAKEAAAKA

SEQ ID NO: 19 - protein, 46 aa, synthetic construct; identical to SEQ ID NO: 18

SEQ ID NO: 20 - protein, 15 aa, synthetic construct
GGGGSGGGGSGGGGS

SEQ ID NO: 21 - protein, 46 aa, synthetic construct; identical to SEQ ID NO: 18

SEQ ID NO: 22 - protein, 5 aa, synthetic construct
GGGGS

SEQ ID NO: 23 - protein, 5 aa, synthetic construct
PAPAP

SEQ ID NO: 24 - protein, 12 aa, synthetic construct
AEAAAKEAAAKA

SEQ ID NO: 25 - protein, 5 aa, synthetic construct
  REGION >5: note = The entire sequence of amino acids 1-5 can be repeated one, two or four times
GGGGS

SEQ ID NO: 26 - protein, 10 aa, synthetic construct
  REGION 1..2: note = The AP residues can be repeated five to seventeen times
APAPAPAPAP

SEQ ID NO: 27 - protein, 17 aa, synthetic construct
VSQTSKLTRAETVFPDV

SEQ ID NO: 28 - protein, 6 aa, synthetic construct
PLGLWA

SEQ ID NO: 29 - protein, 10 aa, synthetic construct
EDVVCCSMSY

SEQ ID NO: 30 - protein, 8 aa, synthetic construct
GGIEGRGS

SEQ ID NO: 31 - protein, 10 aa, synthetic construct
TRHRQPRGWE

SEQ ID NO: 32 - protein, 10 aa, synthetic construct
AGNRVRRSVG

SEQ ID NO: 33 - protein, 9 aa, synthetic construct
RRRRRRRRR

SEQ ID NO: 34 - protein, 4 aa, synthetic construct
GFLG

SEQ ID NO: 35 - protein, 46 aa, synthetic construct; identical to SEQ ID NO: 18

SEQ ID NO: 36 - protein, 42 aa, synthetic construct
MDNVVTEAAEFADLDIDDFDLAVDEELAALSVGGLGNTEVGA

SEQ ID NO: 37 - protein, 14 aa, synthetic construct
  VARIANT 12: note = X can be Y, W, F, or H
SCNCFCYICCSXLI

SEQ ID NO: 38 - protein, 9 aa, synthetic construct
  VARIANT 7: note = X can be Y, W, F or H
SNFYISXLI

SEQ ID NO: 39 - protein, 12 aa, synthetic construct
SGRGKIQASWLI

SEQ ID NO: 40 - protein, 12 aa, synthetic construct
SGANGVKTAWLI

SEQ ID NO: 41 - protein, 16 aa, synthetic construct
  MOD_RES 16: note = Dehydroalanine
VESLTAGHGMTEVGAX

SEQ ID NO: 42 - skipped

SEQ ID NO: 43 - protein, 14 aa, synthetic construct
  VARIANT 3: note = X can be any amino acid
  VARIANT 5: note = X can be any amino acid
  VARIANT 7: note = X can be any amino acid
  VARIANT 12: note = X can be Y, W, F, or H
SCXCXCXICCSXLI

SEQ ID NO: 44 - protein, 9 aa, synthetic construct
  VARIANT 7: note = X can be Y, W, F or H
  VARIANT 2..4: note = X can be any amino acid
SXXXISXLI

SEQ ID NO: 45 - protein, 11 aa, synthetic construct
  VARIANT 3: note = X can be any amino acid
  VARIANT 7: note = X can be any amino acid
  REGION >3: note = The amino acid 3 can be repeated one to three times or absent
  REGION >7: note = The amino acid 7 can be repeated one to three times or absent
SGXRGDXSWLI

SEQ ID NO: 46 - protein, 11 aa, synthetic construct
  VARIANT 3: note = X can be any amino acid
  VARIANT 7: note = X can be any amino acid
  REGION >3: note = The amino acid 3 can be repeated one to three times or absent
  REGION >7: note = The amino acid 7 can be repeated one to three times or absent
CGXRGDXCWLI

SEQ ID NO: 47 - protein, 10 aa, synthetic construct
CGRGDRCWLI

SEQ ID NO: 48 - protein, 12 aa, synthetic construct
CGFRGDAGCWLI

SEQ ID NO: 49 - protein, 12 aa, synthetic construct
CGRGDFVGCWLI

SEQ ID NO: 50 - protein, 13 aa, synthetic construct
CGRGDFVAGCWLI

SEQ ID NO: 51 - protein, 10 aa, synthetic construct
SGRGDRSWLI

SEQ ID NO: 52 - protein, 13 aa, synthetic construct
SGFRGDAGSCWLI

SEQ ID NO: 53 - protein, 12 aa, synthetic construct
SGRGDFVGSWLI

SEQ ID NO: 54 - protein, 13 aa, synthetic construct
SGRGDFVAGSWLI

SEQ ID NO: 55 - protein, 40 aa, synthetic construct
SDLDIVDLDLAVDEELAALSVGGLGNTEVGASGWLGSWVI

SEQ ID NO: 56 - protein, 48 aa, synthetic construct
MRRRGSMDNVVTEAAEFADLDIVDLDLAVDEELAALSVGGLGNTEVGA

SEQ ID NO: 57 - protein, 10 aa, synthetic construct
SGFFGRSWLI

SEQ ID NO: 58 - protein, 9 aa, synthetic construct
SGLGHSWLI

SEQ ID NO: 59 - protein, 9 aa, synthetic construct
SGRANSWLI

SEQ ID NO: 60 - protein, 9 aa, synthetic construct
SGVKFSWLI

SEQ ID NO: 61 - protein, 10 aa, synthetic construct
SGVKADSWLI

SEQ ID NO: 62 - protein, 9 aa, synthetic construct
SGPKGSWLI

SEQ ID NO: 63 - protein, 9 aa, synthetic construct
SGAWRSWLI

SEQ ID NO: 64 - protein, 10 aa, synthetic construct
SGHAAHSWLI

SEQ ID NO: 65 - protein, 10 aa, synthetic construct
SGMPRGSWLI

SEQ ID NO: 66 - protein, 10 aa, synthetic construct
SGNERGSWLI

SEQ ID NO: 67 - protein, 10 aa, synthetic construct
SGKLGFSWLI

SEQ ID NO: 68 - protein, 10 aa, synthetic construct
SGGRENSWLI

SEQ ID NO: 69 - protein, 9 aa, synthetic construct
SGRYHSWLI

SEQ ID NO: 70 - protein, 10 aa, synthetic construct
SGAQQHSWLI

SEQ ID NO: 71 - protein, 10 aa, synthetic construct
SGVKAWSWLI

SEQ ID NO: 72 - protein, 10 aa, synthetic construct
SGHWANSWLI

SEQ ID NO: 73 - protein, 11 aa, synthetic construct
SGKAPGRSWLI

SEQ ID NO: 74 - protein, 11 aa, synthetic construct
SGAGLNRSWLI

SEQ ID NO: 75 - protein, 10 aa, synthetic construct
SGDGWRSWLI

SEQ ID NO: 76 - protein, 11 aa, synthetic construct
SGKGKQASWLI

SEQ ID NO: 77 - protein, 10 aa, synthetic construct
SGIYHNSWLI

SEQ ID NO: 78 - protein, 10 aa, synthetic construct
SGPMHYSWLI

SEQ ID NO: 79 - protein, 10 aa, synthetic construct
SGHQFKSWLI

SEQ ID NO: 80 - protein, 11 aa, synthetic construct
SGIKNDASWLI

SEQ ID NO: 81 - protein, 11 aa, synthetic construct
SGQGRNVSWLI

SEQ ID NO: 82 - protein, 11 aa, synthetic construct
SGRGYALSWLI

SEQ ID NO: 83 - protein, 10 aa, synthetic construct
SGMARYSWLI

SEQ ID NO: 84 - protein, 12 aa, synthetic construct
SGDDRDGGSWLI

SEQ ID NO: 85 - protein, 12 aa, synthetic construct
SGIAKAGQSWLI

SEQ ID NO: 86 - protein, 11 aa, synthetic construct
SGKALEFSWLI

SEQ ID NO: 87 - protein, 11 aa, synthetic construct
SGPRANYSWLI

SEQ ID NO: 88 - protein, 11 aa, synthetic construct
SGFIRNASWLI

SEQ ID NO: 89 - protein, 11 aa, synthetic construct
SGNAWIKSWLI

SEQ ID NO: 90 - protein, 12 aa, synthetic construct
SGYANRAGSWLI

SEQ ID NO: 91 - protein, 12 aa, synthetic construct
SGERGMGKSWLI

SEQ ID NO: 92 - protein, 11 aa, synthetic construct
SGREFANSWLI

SEQ ID NO: 93 - protein, 12 aa, synthetic construct
SGAVRFNGSWLI

SEQ ID NO: 94 - protein, 12 aa, synthetic construct
SGRVALEASWLI

SEQ ID NO: 95 - protein, 11 aa, synthetic construct
SGGYRPHSWLI

SEQ ID NO: 96 - protein, 11 aa, synthetic construct
SGRYAGWSWLI

SEQ ID NO: 97 - protein, 12 aa, synthetic construct
SGNMHPGKSWLI

SEQ ID NO: 98 - protein, 11 aa, synthetic construct
SGEMHQHSWLI

SEQ ID NO: 99 - protein, 12 aa, synthetic construct
SGQPQRLGSWLI

SEQ ID NO: 100 - protein, 12 aa, synthetic construct
SGQDVANRSWLI

SEQ ID NO: 101 - protein, 11 aa, synthetic construct
SGLKFYHSWLI

SEQ ID NO: 102 - protein, 12 aa, synthetic construct
SGGWRVAHSWLI

SEQ ID NO: 103 - protein, 12 aa, synthetic construct
SGFDRAGHSWLI

SEQ ID NO: 104 - protein, 12 aa, synthetic construct
SGIAKEVQSWLI

SEQ ID NO: 105 - protein, 11 aa, synthetic construct
SGQYKFRSWLI

SEQ ID NO: 106 - protein, 12 aa, synthetic construct
SGHDNQARSWLI

SEQ ID NO: 107 - protein, 12 aa, synthetic construct
SGYRRRAGSWLI

SEQ ID NO: 108 - protein, 12 aa, synthetic construct
SGPKRWGHSWLI

SEQ ID NO: 109 - protein, 12 aa, synthetic construct
SGLQHGKYSWLI

SEQ ID NO: 110 - protein, 12 aa, synthetic construct
SGDAMRWASWLI

SEQ ID NO: 111 - protein, 12 aa, synthetic construct
SGYHFHNVSWLI

SEQ ID NO: 112 - protein, 12 aa, synthetic construct
SGHYKPQASWLI

SEQ ID NO: 113 - protein, 11 aa, synthetic construct
SGYDRQYSWLI

SEQ ID NO: 114 - protein, 11 aa, synthetic construct
SGQGRNRSWLI

SEQ ID NO: 115 - protein, 11 aa, synthetic construct
SGKALERSWLI

SEQ ID NO: 116 - protein, 11 aa, synthetic construct
SGREFARSWLI

SEQ ID NO: 117 - protein, 11 aa, synthetic construct
SGGYRPRSWLI

SEQ ID NO: 118 - protein, 11 aa, synthetic construct
SGRYAGRSWLI

SEQ ID NO: 119 - protein, 12 aa, synthetic construct
SGIAKEVRSWLI

SEQ ID NO: 120 - protein, 12 aa, synthetic construct
SGLQHGKRSWLI

SEQ ID NO: 121 - protein, 12 aa, synthetic construct
SGDAMRWRSWLI

SEQ ID NO: 122 - protein, 12 aa, synthetic construct
SGYHFHNRSWLI

SEQ ID NO: 123 - protein, 12 aa, synthetic construct
SGHYKPQRSWLI

SEQ ID NO: 124 - protein, 11 aa, synthetic construct
SGYDRQRSWLI

SEQ ID NO: 125 - DNA, 28 bp, synthetic construct
  source 1..28: mol_type = other DNA
gaggaagagttggcgaaagatccacgta

SEQ ID NO: 126 - DNA, 20 bp, synthetic construct
  source 1..20: mol_type = other DNA
taatacgactcactataggg

SEQ ID NO: 127 - DNA, 19 bp, synthetic construct
  source 1..19: mol_type = other DNA
gctagttattgctcagcgg

SEQ ID NO: 128 - DNA, 20 bp, synthetic construct
  source 1..20: mol_type = other DNA
gagcgcattcagggtctgtt

SEQ ID NO: 129 - DNA, 20 bp, synthetic construct
  source 1..20: mol_type = other DNA
tgctttgtgcagcaggttcc

SEQ ID NO: 130 - DNA, 20 bp, synthetic construct
  source 1..20: mol_type = other DNA
gtgctggtattcactgaagc

SEQ ID NO: 131 - DNA, 20 bp, synthetic construct
  source 1..20: mol_type = other DNA
tgttgactcgttcccgctca

SEQ ID NO: 132 - DNA, 53 bp, synthetic construct
  source 1..53: mol_type = other DNA
cttccaatcccgtcgtcgtggatccatggacaacgtcgtgactgaagccgccg

SEQ ID NO: 133 - DNA, 54 bp, synthetic construct
  source 1..54: mol_type = other DNA
cagtggtggtggtggtggtgctcgagtcaaatcacccagctgcccagccaaccg

SEQ ID NO: 134 - DNA, 27 bp, synthetic construct
  source 1..27: mol_type = other DNA
aaggatccatggacaatgcggctacgg

SEQ ID NO: 135 - DNA, 37 bp, synthetic construct
  source 1..37: mol_type = other DNA
aactcgagtcagattaaccaggaacgaccaaaaaatc

SEQ ID NO: 136 - DNA, 53 bp, synthetic construct
  source 1..53: mol_type = other DNA
cagaatacgtggtagagattggactgccatgactcgagtcgacaagcttgcgg

SEQ ID NO: 137 - DNA, 42 bp, synthetic construct
  source 1..42: mol_type = other DNA
gtgggcggtgccggcatggatccggattggaagtacaggttc

SEQ ID NO: 138 - DNA, 42 bp, synthetic construct
  source 1..42: mol_type = other DNA
gaacctgtacttccaatccggatccatgccggcaccgcccac

SEQ ID NO: 139 - DNA, 53 bp, synthetic construct
  source 1..53: mol_type = other DNA
ccgcaagcttgtcgactcgagtcatggcagtccaatctctaccacgtattctg

SEQ ID NO: 140 - DNA, 30 bp, synthetic construct
  source 1..30: mol_type = other DNA
aaggatccatgttacgtcaccgcggtcaag

SEQ ID NO: 141 - DNA, 32 bp, synthetic construct
  source 1..32: mol_type = other DNA
aactcgagttatggtgtctcttgctctggcac

SEQ ID NO: 142 - DNA, 26 bp, synthetic construct
  source 1..26: mol_type = other DNA
aaggatccatgacacgtaccccgccg

SEQ ID NO: 143 - DNA, 30 bp, synthetic construct
  source 1..30: mol_type = other DNA
aactcgagtcagcgacggtcgttgacaaac

SEQ ID NO: 144 - DNA, 53 bp, synthetic construct
  source 1..53: mol_type = other DNA
ggatctgagaacctgtacttccaatccgatttggacattgttgatctggacct

SEQ ID NO: 145 - DNA, 54 bp, synthetic construct; identical to SEQ ID NO: 133
  source 1..54: mol_type = other DNA

SEQ ID NO: 146 - DNA, 53 bp, synthetic construct; identical to SEQ ID NO: 144
  source 1..53: mol_type = other DNA

SEQ ID NO: 147 - DNA, 53 bp, synthetic construct
  source 1..53: mol_type = other DNA
cagtggtggtggtggtggtgctcgagtcaaatcacgccgctgcccagccaacc

SEQ ID NO: 148 - DNA, 53 bp, synthetic construct; identical to SEQ ID NO: 144
  source 1..53: mol_type = other DNA

SEQ ID NO: 149 - DNA, 49 bp, synthetic construct
  source 1..49: mol_type = other DNA
cagtggtggtggtggtggtgctcgagtcaaatgccccagctgcccagcc

SEQ ID NO: 150 - DNA, 53 bp, synthetic construct; identical to SEQ ID NO: 144
  source 1..53: mol_type = other DNA

SEQ ID NO: 151 - DNA, 48 bp, synthetic construct
  source 1..48: mol_type = other DNA
gctgggcagctgggtgggctgactcgagcaccaccaccaccaccactg

SEQ ID NO: 152 - DNA, 47 bp, synthetic construct
  source 1..47: mol_type = other DNA
gattttttggtcgttgtgggttaatctgactcgagcaccaccaccac

SEQ ID NO: 153 - DNA, 42 bp, synthetic construct
  source 1..42: mol_type = other DNA
gagtcagattaacccacaacgaccaaaaaatccgcacgcacc

SEQ ID NO: 154 - DNA, 39 bp, synthetic construct
  source 1..39: mol_type = other DNA
gttgttggttaggctgactcgagcaccaccaccaccacc

SEQ ID NO: 155 - DNA, 41 bp, synthetic construct
  source 1..41: mol_type = other DNA
gctcgagtcagcctaaccaacaacgaccaaaaaatccgcac

SEQ ID NO: 156 - DNA, 56 bp, Thermobispora bispora
  source 1..56: mol_type = other DNA
aattcctgcagtaatacgactcactataggtcccgtcgtctagaggcctaggacgc

SEQ ID NO: 157 - DNA, 58 bp, Thermobispora bispora
  source 1..58: mol_type = other DNA
  modified_base 1: mod_base = OTHER; note = Uracil
  modified_base 1..2: mod_base = OTHER; note = 2-Prime-O-methyl nucleotides
tggtagtcccgagcggattcgaaccgccgttaccgccttgagagggcggcgtcctagg

SEQ ID NO: 158 - protein, 11 aa, synthetic construct
GASGWLGSWVI

SEQ ID NO: 159 - protein, 31 aa, synthetic construct
SDLDIVDLDLAVDEELAALSVGGLGNTEVGA

SEQ ID NO: 160 - protein, 40 aa, synthetic construct
SDLDIVDLDLAVDEELAALSVGGLGNTEVGASGWLGSGVI

SEQ ID NO: 161 - protein, 40 aa, synthetic construct
SDLDIVDLDLAVDEELAALSVGGLGNTEVGASGWLGSWGI

SEQ ID NO: 162 - protein, 40 aa, synthetic construct
SDLDIVDLDLAVDEELAALSVGGLGNTEVGASGWLGSWVG

SEQ ID NO: 163 - protein, 6 aa, synthetic construct
DDRDGG

SEQ ID NO: 164 - protein, 6 aa, synthetic construct
IAKAGQ

SEQ ID NO: 165 - protein, 6 aa, synthetic construct
YANRAG

SEQ ID NO: 166 - protein, 6 aa, synthetic construct
ERGMGK

SEQ ID NO: 167 - protein, 6 aa, synthetic construct
AVRFNG

SEQ ID NO: 168 - protein, 6 aa, synthetic construct
RVALEA

SEQ ID NO: 169 - protein, 6 aa, synthetic construct
NMHPGK

SEQ ID NO: 170 - protein, 6 aa, synthetic construct
QPQRLG

SEQ ID NO: 171 - protein, 6 aa, synthetic construct
QDVANR

SEQ ID NO: 172 - protein, 6 aa, synthetic construct
GWRVAH

SEQ ID NO: 173 - protein, 6 aa, synthetic construct
FDRAGH

SEQ ID NO: 174 - protein, 6 aa, synthetic construct
IAKEVQ

SEQ ID NO: 175 - protein, 6 aa, synthetic construct
HDNQAR

SEQ ID NO: 176 - protein, 6 aa, synthetic construct
YRRRAG

SEQ ID NO: 177 - protein, 6 aa, synthetic construct
PKRWGH

SEQ ID NO: 178 - protein, 6 aa, synthetic construct
LQHGKY

SEQ ID NO: 179 - protein, 6 aa, synthetic construct
DAMRWA

SEQ ID NO: 180 - protein, 6 aa, synthetic construct
YHFHNV

SEQ ID NO: 181 - protein, 6 aa, synthetic construct
HYKPQA

SEQ ID NO: 182 - protein, 6 aa, synthetic construct
IAKEVR

SEQ ID NO: 183 - protein, 6 aa, synthetic construct
LQHGKR

SEQ ID NO: 184 - protein, 6 aa, synthetic construct
DAMRWR

SEQ ID NO: 185 - protein, 6 aa, synthetic construct
YHFHNR

SEQ ID NO: 186 - protein, 6 aa, synthetic construct
HYKPQR

SEQ ID NO: 187 - protein, 42 aa, synthetic construct
MDNAATEATEFADLDIVNLDLPIDEELAAVSVGGLGNTEVGA

SEQ ID NO: 188 - protein, 42 aa, synthetic construct
MDNVVTEAAEFADLDIVDLDLAVDEELAALSVGGLGNTEVGA

SEQ ID NO: 189 - protein, 9 aa, synthetic construct
SGWLGSWVI

SEQ ID NO: 190 - skipped

SEQ ID NO: 191 - protein, 11 aa, synthetic construct
  MOD_RES 8: note = Dehydroalanine
GASGWLGAWVI

SEQ ID NO: 192 - DNA, 76 bp, Micromonospora rosaria
  source 1..76: mol_type = other DNA
ggccccgtcgtctagcggcccaggacgccgccctctcaaggcggtagcgccggttcgaatccggtcggggctacca

SEQ ID NO: 193 - DNA, 76 bp, Thermobispora bispora
  source 1..76: mol_type = other DNA
ggtcccgtcgtctagaggcctaggacgccgccctctcaaggcggtaacggcggttcgaatccgctcgggactacca

SEQ ID NO: 194 - protein, 27 aa, synthetic construct
SGFFGNGNGNGNGNGNGNGNGNRSWLI

SEQ ID NO: 195 - protein, 9 aa, synthetic construct
SNFYISWLI

SEQ ID NO: 196 - protein, 51 aa, synthetic construct
MATNEPFHLDLDSLDVMTVELPGEDLVKALGMGLGNTEVGASGYMRTSWVV

SEQ ID NO: 197 - protein, 52 aa, synthetic construct
MEDMAVEATEFADLDIVDLDLPIDEELAAVSIGGLGNTEVGASGFWGRSWLI

SEQ ID NO: 198 - protein, 52 aa, synthetic construct
MATEEPLRLDLDTLDVMTVELPGEDLVKALGMGLGNTEVGASGITGRTSWLI

SEQ ID NO: 199 - protein, 52 aa, synthetic construct
MDLHEEALDLDALDVATVELPGSEVLVEAVAMGLGNTEIGASGCTSGKSWLI

SEQ ID NO: 200 - protein, 52 aa, synthetic construct
MATNEPLRLDLDSLDVMTVELPGEDLVQALGMGLGNTEVGASGLTGRTSWLI

SEQ ID NO: 201 - protein, 58 aa, synthetic construct
MAMHEEALNLDALDLDSLDVATVELPGSDLLVEAVTMGLGNTEVGASGAWTSRTSWLV

SEQ ID NO: 202 - protein, 58 aa, synthetic construct
MAMHEEALNLDALDLDSLDVATVELPGSDVLVEAVTMGLGNTEVGASGAWTSRTSWLV

SEQ ID NO: 203 - protein, 58 aa, synthetic construct
MAMHEEVLDLDALDLDSLDVATVELPGSEVLVEAVTMGLGNTEIGASGTWTSSKSWLV

SEQ ID NO: 204 - protein, 78 aa, synthetic construct
MDEIVDRAYGGDRHRYGPLRRVRMGVOMATNEPLRLDLDALDVMTVELPGEDLVKALGMGLGNTEVGASGIARTSWLI

SEQ ID NO: 205 - protein, 65 aa, synthetic construct
MTIVPEIARETGEOMANDEPLHLDLNSLDVTTVELPGEDLVEALGMGLGNTEVGASGIGRTSWLI

SEQ ID NO: 206 - protein, 13 aa, synthetic construct
SGFFGNGGRSWLI

SEQ ID NO: 207 - protein, 14 aa, synthetic construct
SGFFGNGGNRSWLI

SEQ ID NO: 208 - protein, 15 aa, synthetic construct
SGFFGNGNGNRSWLI

SEQ ID NO: 209 - protein, 17 aa, synthetic construct
SGFFGNGNGNGNRSWLI

SEQ ID NO: 210 - protein, 20 aa, synthetic construct
SGFFGNGNGNGGNGNRSWLI

SEQ ID NO: 211 - protein, 22 aa, synthetic construct
SGFFGNGNGNGGNGNGNRSWLI

SEQ ID NO: 212 - protein, 25 aa, synthetic construct
SGFFGNGNGNGNGNGNGNGNRSWLI

SEQ ID NO: 213 - protein, 27 aa, synthetic construct
SGFFGNGNGNGNGNGNGNGNGNRSWLI

SEQ ID NO: 214 - protein, 25 aa, synthetic construct
  VARIANT 5..16: note = X can be any amino acid except Cys, Ser or Thr
SGFFXXXXXXXXXXXXGNGNRSWLI

SEQ ID NO: 215 - protein, 25 aa, synthetic construct
SGFFHGQNMIEYWKPAGNGNRSWLI

SEQ ID NO: 216 - protein, 25 aa, synthetic construct
SGFFWDIAFVPEYMGNGNGNRSWLI

SEQ ID NO: 217 - protein, 25 aa, synthetic construct
SGFFNEKQVGAIRMWPGNGNRSWLI

SEQ ID NO: 218 - protein, 25 aa, synthetic construct
SGFFRWNKIYLMEGPQGNGNRSWLI

SEQ ID NO: 219 - protein, 25 aa, synthetic construct
SGFFYLIHKGPNRAQDGNGNRSWLI

SEQ ID NO: 220 - protein, 25 aa, synthetic construct
SGFFVQNLMHPDWIEAGNGNRSWLI

SEQ ID NO: 221 - protein, 25 aa, synthetic construct
SGFFNQRHDKIEFLWGGNGNRSLWI

SEQ ID NO: 222 - protein, 25 aa, synthetic construct
SGFFRNILGQEKPDFVGNGNRSWLI

SEQ ID NO: 223 - protein, 25 aa, synthetic construct
SGFFGWPIEVMYQKNLGNGNRSWLI

SEQ ID NO: 224 - protein, 49 aa, synthetic construct
MDLNDLPMDVFELADSGVAVESLTAGHGMTEVGASCNCFCYICCSCSSA

SEQ ID NO: 225 - protein, 56 aa, synthetic construct
MDNVVTEAAEFADLDIDDFDLAVDEELAALSVGGLGNTEVGASCNCFCYICCSWLI

SEQ ID NO: 226 - protein, 62 aa, synthetic construct
MRRRGSMDNVVTEAAEFADLDIDDFDLAVDEELAALSVGGLGNTEVGASCNCFCYICCSWLI

SEQ ID NO: 227 - DNA, 168 bp, synthetic construct
  source 1..168: mol_type = other DNA
ggatccatggacaacgtcgtgactgaagccgccgagttcgcggatttggacattgttgatctggaccttgcagtagatgaagaacttgccgctttgagcgtcgggggtttgggtaatacagaggtcggtgcgagcggttggctgggcagctgggtgatttgactcgag

SEQ ID NO: 228 - DNA, 171 bp, synthetic construct
  source 1..171: mol_type = other DNA
ggatccatggacaatgcggctacggaagctacggagtttgcagaccttgatattgtgaacctggacctccctatcgacgaagaacttgccgctgtcagcgtcgggggtttgggtaatacagaggtcggtgcgtccggattttttggtcgttcctggttaatctgactcgag

SEQ ID NO: 229 - DNA, 2602 bp, synthetic construct
  source 1..2602: mol_type = other DNA
ggatccatgccggcaccgcccacggcagcgacgggcccagttgtcgtggtgcgtgtcgcggcattgccgacgcaggccttagatgatactgcggcgcccgccagctgggcaatggtacaggctattctggcgtcccgccgtcgtgtagcggaagccggcgcccgtcttgcggatgaagttcacggtttcattgccgatccggcgcttgcgggtgcccgtcctgaactggttgctttacgccgtgccttacataatcgtcgccgtcctggccatcgcgcatggcctggcggccatgctgaattacttccggcccggtttcgcagtgagttaaccggttggacggctgagttggaccgtgcggccgcgctgacccgtcagctgccagatctgctggatgctgagtggacacgctctttgcgcgcgctgcgccgttggagtgcgaccgaagttttcgaatttggcttattgcagtcatctgaagacctgttgcatgcacttctgcgttggcgtgatcagccggaaggaaccccaccgcgtgctcaggtcgcactgcgtctggcgaaatatctggcgcgcgcagtggcaaagacctctccgcaagccacctttatgatgtctggcctgtgccgctggagtgatgcaccgaccgcggtgcagcctacaggtcgctgggcgtggcgctccgtagtcgagccgaacgtcccgctcctgcaggcgctgactcgccgtctgccagctgattcaacggccaccggtcgcctgatggtggtaaccacgccggacgcggacgtggctgatggtcggatctggtttgctcatccgcgtcgtgagcgcattcagggtctgttactcacgcctccggtgcgcactgcgatcgaagcagttggcggcggggccccactggctgaagtaggacgccgccttgcgggcgatacggccgtcacgttcgatcgtctcgtcgaggtcggcttattcgaaatccagccaccttttgccgatcaagcagcggatcaccttcacgacttacgcgcatggctggggcaggaagcacctgcggccggaccgctgcgtgcgttagcggacgccctggatgcctaccctgcaccaggtaccgatgcgtctcgccgtcgtgagctccaccgccgtattcgcacagaatttcaggccgtccacgggcctgttggcgatacgccgctgcctgcgcgcaacctgtttcgcgaaaacgccgtattcaccgctgaacccgtgcgcctgggcggggtgccgtggcggccggcggtcgctgatttgcacgtagttcatcgtttactcgcgctgtttcaacattctaccgtagtccgcctggtggcacatcgtgttgtcgtggatcgctatggtgtagatacgcgtgttgggttactcgaactgcatcgggaactgagccgtctggcggttgatgcggagcccggcacagccgtggcagaattggccggcctgttactgctgcgtccggggcagcctccggacccagctcgcagcacccttccggaggtccgttggctgcaccgcttacgtcaacaagcattggctgctctttgggctgggcctgaaacgccgccggagwggcgtgacggtggatccggctcgcgtgcgtgctctggccgatggctggccgccatgggtccgtccggcgccggcgcttgcatgctttgtgcagcaggttccggcagcggacggcttgcgcctggtattgaacggcgtgggtgcgggacacggcgctgttcgttcacgcgtccatcgcttactggcaaaggtggcgccagaaggtgccggcaccggagagctgcctaaagccacgccgacccctgcgccggccggctacctggcggagattaccggcttgtttggttcgaccgtgaacctgcgtagcccggctgtggattttgaaatttcgcatcccggtactgtatctgcacgccctccgcagcatcgtatcgccctcgacgatctggttgttactgcggatggtacgacccgtagtcttcgcctgcgtcgccgttcagatgatggcgaggtacgcattgcgcatacaggtatgatggcaggcgctcggttgccgcgcttggcgcggttcctgattcacttgtttggcgatgtcccgaacgccgccgcgcctacggcgcagatctttgccggtaccccggagcaccagccgggcgcagtgcggcgccaaccgcgcctgtctattggccgtgtaactgtaagtcgcgcttcatggcgcgtccgtgccgacctcgttccgcgtcgtgctaaaggcgtcgatgatgcgggttacctgttggccctggctgcatggttcgccgatcaccgcatcccgccacgcacctatgtaaccgcaaccggcccgacgccgcccggtacgcctccggtgccagtaaaaccgatctatttggacctggccaatctgctgctggtgcgcctgtttgaacggctgctgcgtgaaccggatagcgtgctggtattcactgaagcgctgcctcagctggctgatgctccatgttttggaacggaaggacgccatgtaacagaatacgtggtagagattggactgccatgactcgag

SEQ ID NO: 230 - DNA, 1068 bp, synthetic construct
  source 1..1068: mol_type = other DNA
ggatccatgttacgtcaccgcggtcaagcgcgcgatcgtgttcgtcgtcgcgatcgtccgcctgtaacaccaccgcccacttggcttagttggcacgcgttccaccacggtgaccatacgcgcctgattaccggtgcagtagacccgctcgttgcgagtctgcaggcagacggccgtattgcacagttcttctttatccgttactgggaaggcggtcctcacctgcgcttacgtctgttgcccactcacgcggagtactcaactgaagtggcggcacgcgctcgttcggccttagagcgtcatcttgcgcgctttccgtcgcctccctccgcgcctgcgggccaatatgccgctttggctgagcgctacgcgcgcttagaagggttagccgatcatgaccgcctgcgcccgccggatgtagttgaggcagtcccttaccacccagagtaccccgtgtttggcggccgcgccgccactcgtgccgtagagcgtcacttcacggactctagccgcctggcgttgagcgtccttgcgcgtcgcgcgccccaacaccgtagccgcctcgcagcgctggcccttgcctcgacccttgcggcgtggcagccagaccgtcgccgcttagcgcacctgttgactcgttcccgctcattttgggagccagccgagggccgcggtctgcgtcgcgagagctaccaacgtcaacgcgcagccctgcgtcgtctcgtttcgggctgttggccgccgacccctggcccaccaccccaagatcccgatccgttggccgctgcctggtctgatagcgtccaccgtcttcacgcggagctgacagacctgcgtcgtgccggggatttccatcctgacttgcgtgcggtggctacgcgctttgccgccgataccggtcccacgcgcgacgagcaaggtttactggttgtgcttctccgctgcgtgcaccttctgtgtaatcgcttaggtcttggtgtggaccaggagacccaattacgttacttagtgtgtgccgcctgtgcggacatcgaggccgtgccagagcaagagacaccataactcgag

SEQ ID NO: 231 - DNA, 1112 bp, synthetic construct
  source 1..1112: mol_type = other DNA
ggatccatgacacgtaccccgccgcatcgcgaagaaccgggttggcatagtatccaaattcggtatcatgctgagaacaaagacgatttgattctggatgcggttcacccattacttacggccctggccgacgcggttgaccaacctcacctgttacgccactggcgccggggcccgcacctccgcatcaacctgcgtaccaccccgcagctctgggcagaagttgttcgcccgaccgcccagcgcgatcttaccgcgtacctgcatgataacccatcaaccgccagcttacgcgaagccgaacatctggcggcccaccgccgcctggcagtgcgtgaggcggatcatggacctctgactccctggtaccctgataatacagttcaatttgaaccgcatgaagatcggcagcatgttctgggtagcccggcgcttgcaggtttgctggcggatggctatgcacgctcgactgccttaaccgtggatacgctggctggcgttcgttctggagcggtggatcgcgtagggcatgctcttgatttaatgtttgcatttggtcatctgtctgtgccgccgatttctcgcggctacatgtccttccgctcacatgtggaaagttttttgggttataccgctgatccggatgccgtccgctcgaccttcgatgagcgttaccatcggcaccggcccgcgctgcgcgacctttacatactacgcgcgccattgttgcgggaagtcgccgtgatccgctggtggaggcatggctggaaatcgtgcgtcgccagaaagctctggcggaaccgctgtttacgtctggcgccattgatctggaccatcttgatcagcgcgggatggaattacgccaccgcatcgagtttcacgatattctgcgcgagaccactgcacatcgtcgtgaggtactttcacaggtgtggtttcgctgtcatcgcttagccattaaccaactgtacagccatttgtctcgcatcggtatcgtgcccactcagcgctacttactctgccatctggtagctcgcaccgtggaggaagaatatgatatcagtccggtggcgctcgcccgtcagtttgtcaacgaccgtcgctgactcgag

SEQ ID NO: 232 - DNA, 1365 bp, Thermobispora bispora
  source 1..1365: mol_type = other DNA
atgattcgtgtgcgtttcgcaccgtctccgaccggcatgttccacgtcggtggtgcccgctctgccctgtacaactgggccttcgccctgcgtcacggcggcaccttcgtcctgcgcatcgaggacaccgaccagtctcgcaaccgtccggagtggaccgagggcatcatctctgccctggcctggctgggcatcagcaaggagtctccgcacttcgagggtccgtacttccagtcctcttacgagaagctgcaccgcgaggcggcccagcgtctgctgtctgagggtaaggcatactactgcacctgcacccgtgacgacgtgaaggcccgtaccggctccgagtacccagggttacgacggttactgccgtgatcgtggcctgaccgagggtgcgatccgtttccgcaccccggacgacggtgttaccgttgtggacgacgtggtccgtggccgtgtggagttcccgaacgcggcgatggaggacttcgtcatcgtgcgcggtgacggctctccgctgttcatcctggccaacgttgtggacgacatggagatgcgtatcacccacgtgatccgtgccgaagagcatctgagcaacactccgaagcagcaactgctgtgggaagctctgggtgctcgtccgccggtgtgggctcacgtcccggtgatcgtcaacgagaagcgtcagaagctgtccaagcgtcgtgacaaggtcgcgctggagtcctaccgcgaggagggctacctgccggaggcgatggtcaactacctgatgctgctgggctggggtccgggtggcgatcgtgagatcatgccgtggccgagatggtgcagtgcttccgtctggaggacgtcaacccgtctccggcgttcttcgacgagaagaagctgcgcgttcaacggtgagtacatccgtccctgtctccggaggacttcgccgcccgttgcgagccgtacctggatccgtcttgggatcgtaaggtgttcgcacgcgtcgctccgctggcacagacccgtatctccgtgctgtctgagatccgtgactacgtagacttcctgttcctggacgaaccggttttcgatcaggcatcctgggacaaggctatgaagccgggtgcagccgagatcctggccgagtacgctgagcgtctggagtctgtggagtggaccccggaagcgctgaagactgcgctggaggaggtgggtgcagctcacggtctcaaactggctaaagctcaggctccggttcgtgtggctgtaaccggtcgtactgtcggtctgccgctgttcgaatctatcgaggtgctgggtcgtgagcgctctctggcacgtgtgcgcgctgcgctggccaagctgtccgcttaa

SEQ ID NO: 233 - DNA, 1245 bp, synthetic construct
  source 1..1245: mol_type = other DNA
atgggcagcagccatcatcatcatcatcacagcagcggcctggtgccgcgcggcagccatatgaaaatcgaagaaggtaaactggtaatctggattaacggcgataaaggctataacggtctcgctgaagtcggtaagaaattcgagaaagataccggaattaaagtcaccgttgagcatccggataaactggaagagaaattcccacaggttgcggcaactggcgatggccctgacattatcttctgggcacacgaccgctttggtggctacgctcaatctggcctgttggctgaaatcaccccggacaaagcgttccaggacaagctgtatccgtttacctgggatgccgtacgttacaacggcaagctgattgcttacccgatcgctgttgaagcgttatcgctgatttataacaaagatctgctgccgaacccgccaaaaacctgggaagagatcccggcgctggataaagaactgaaagcgaaaggtaagagcgcgctgatgttcaacctgcaagaaccgtacttcacctggccgctgattgctgctgacgggggttatgcgttcaagtatgaaaacggcaagtacgacattaaagacgtgggcgtggataacgctggcgcgaaagcgggtctgaccttcctggttgacctgattaaaaacaaacacatgaatgcagacaccgattactccatcgcagaagctgcctttaataaaggcgaaacagcgatgaccatcaacggcccgtgggcatggtccaacatcgacaccagcaaagtgaattatggtgtaacggtactgccgaccttcaagggtcaaccatccaaaccgttcgttggcgtgctgagcgcaggtattaacgccgccagtccgaacaaagagctggcgaaagagttcctcgaaaactatctgctgactgatgaaggtctggaagcggttaataaagacaaaccgctgggtgccgtagcgctgaagtcttacgaggaagagttggcgaaagatccacgtattgccgccaccatggaaaacgcccagaaaggtgaaatcatgccgaacatcccgcagatgtccgctttctggtatgccgtgcgtactgcggtgatcaacgccgccagcggtcgtcagactgtcgatgaagccctgaaagacgcgcagactaattcgagctcccaccatcaccatcaccacgcgaattcggtaccgctggttccgcgtggatctgagaacctgtacttccaatcc

SEQ ID NO: 234 - DNA, 1872 bp, Thermobispora bispora
  source 1..1872: mol_type = other DNA
atgattcgtgtgcgtttcgcaccgtctccgaccggcatgttccacgtcggtggtgcccgctctgccctgtacaactgggccttcgccctgcgtcacggcggcaccttcgtcctgcgcatcgaggacaccgaccagtctcgcaaccgtccggagtggaccgagggcatcatctctgccctggcctggctgggcatcagcaaggagtctccgcacttcgagggtccgtacttccagtcctcttacgagaagctgcaccgcgaggcggcccagcgtctgctgtctgagggtaaggcatactactgcacctgcacccgtgacgacgtgaaggcccgtaccggctccgagtaccagggttacgacggttactgccgtgatcgtggcctgaccgagggtgcgatccgtttccgcaccccggacgacggtgttaccgttgtggacgacgtggtccgtggccgtgtggagttcccgaacgcggcgatggaggacttcgtatcgtgcgcggtgacggctctccgctgttcatcctggccaacgttgtggacgacatggagatgcgtatcacccacgtgatccgtgccgaagagcatctgagcaacactccgaagcacaactgctgtgggaagctctgggtgctcgtccgccggtgtgggctcacgtcccggtgatcgtcaacgagaagcgtcagaagctgtccaagcgtcgtgacaaggtcgcgctggagtcctaccgcgaggagggctacctgccggaggcgatggtcaactacctgatgctgctgggctggggtccgggtggcgatcgtgagatcatgccgtggccggagatggtgcagtgcttccgtctggaggacgtcaacccgtctccggcgttcttcgacgagaagaagctgcgcgcgttcaacggtgagtacatccgtgccctgtctccggaggacttcgccgcccgttgcgagccgtacctggatccgtcttgggatcgtaaggtgttcgcacgcgtcgctccgctggcacagacccgtatctccgtgctgtctgagatccgtgactacgtagacttcctgttcctggacgaaccggttttcgatcaggcatcctgggacaaggctatgaagccgggtgcagccgagatcctggccgagtacgctgagcgtctggagtctgtggagtggaccccggaagcgctgaagactgcgctggaggaggtgggtgcagctcacggtctcaaactggctaaagctcaggctccggttcgtgtggctgtaaccggtcgtactgtcggtctgccgctgttcgaatctatcgaggtgctgggtcgtgagcgctctctggcacgtgtgcgcgctgcgctggccaagctgtccgcttaaactactttatgtagtctccgccgtgtagcaagaaattgagaagttaatacgactcactataggtcccgtcgtctagaggcctaggacgccgccctctcaaggcggtaacggcggttcgaatccgctcgggactaccaatccttagcgaaagctaaggattttttttaaatttgcacggcaaatttgaattcgaggtttaactacatgttattaatacgactcactataggtcccgtcgtctagaggcctaggacgccgccctctcaaggcggtaacggcggttcgaatccgctcgggactaccaatccttagcgaaagctaaggattttttttaaattctgaatgtatcgaatatgttcgagcttaaatcaaaaccaatttgttaatacgactcactataggtcccgtcgtctagaggcctaggacgccgccctctcaaggcggtaacggcggttcgaatccgctcgggactaccaatccttagcgaaagctaaggatttttttta

SEQ ID NO: 235-236 - skipped (2 entries)

SEQ ID NO: 237 - protein, 52 aa, synthetic construct
MDNAATEATEFADLDIVNLDLPIDEELAAVSVGGLGNTEVGASGFFGRSWLI

SEQ ID NO: 238 - protein, 51 aa, synthetic construct
MDNVVTEAAEFADLDIVDLDLAVDEELAALSVGGLGNTEVGASGWLGSWVI

SEQ ID NO: 239 - protein, 4 aa, synthetic construct
FFGR

SEQ ID NO: 240 - protein, 4 aa, synthetic construct
VKAD

SEQ ID NO: 241 - protein, 4 aa, synthetic construct
HAAH

SEQ ID NO: 242 - protein, 4 aa, synthetic construct
MPRG

SEQ ID NO: 243 - protein, 4 aa, synthetic construct
NERG

SEQ ID NO: 244 - protein, 4 aa, synthetic construct
KLGF

SEQ ID NO: 245 - protein, 4 aa, synthetic construct
GREN

SEQ ID NO: 246 - protein, 4 aa, synthetic construct
AQQH

SEQ ID NO: 247 - protein, 4 aa, synthetic construct
VKAW

SEQ ID NO: 248 - protein, 4 aa, synthetic construct
HWAN

SEQ ID NO: 249 - protein, 5 aa, synthetic construct
KAPGR

SEQ ID NO: 250 - protein, 5 aa, synthetic construct
AGLNR

SEQ ID NO: 251 - protein, 4 aa, synthetic construct
DGWR

SEQ ID NO: 252 - protein, 5 aa, synthetic construct
KGKQA

SEQ ID NO: 253 - protein, 4 aa, synthetic construct
IYHN

SEQ ID NO: 254 - protein, 4 aa, synthetic construct
PMHY

SEQ ID NO: 255 - protein, 4 aa, synthetic construct
HQFK

SEQ ID NO: 256 - protein, 5 aa, synthetic construct
IKNDA

SEQ ID NO: 257 - protein, 5 aa, synthetic construct
QGRNV

SEQ ID NO: 258 - protein, 5 aa, synthetic construct
RGYAL

SEQ ID NO: 259 - protein, 4 aa, synthetic construct
MARY

SEQ ID NO: 260 - protein, 5 aa, synthetic construct
KALEF

SEQ ID NO: 261 - protein, 5 aa, synthetic construct
PRANY

SEQ ID NO: 262 - protein, 5 aa, synthetic construct
FIRNA

SEQ ID NO: 263 - protein, 5 aa, synthetic construct
NAWIK

SEQ ID NO: 264 - protein, 5 aa, synthetic construct
REFAN

SEQ ID NO: 265 - protein, 5 aa, synthetic construct
GYRPH

SEQ ID NO: 266 - protein, 5 aa, synthetic construct
RYAGW

SEQ ID NO: 267 - protein, 5 aa, synthetic construct
EMHQH

SEQ ID NO: 268 - protein, 5 aa, synthetic construct
LKFYH

SEQ ID NO: 269 - protein, 5 aa, synthetic construct
QYKFR

SEQ ID NO: 270 - protein, 5 aa, synthetic construct
YDRQY

SEQ ID NO: 271 - protein, 5 aa, synthetic construct
QGRNR

SEQ ID NO: 272 - protein, 5 aa, synthetic construct
KALER

SEQ ID NO: 273 - protein, 5 aa, synthetic construct
REFAR

SEQ ID NO: 274 - protein, 5 aa, synthetic construct
GYRPR

SEQ ID NO: 275 - protein, 5 aa, synthetic construct
RYAGR

SEQ ID NO: 276 - protein, 5 aa, synthetic construct
YDRQR

SEQ ID NO: 277 - protein, 9 aa, synthetic construct
  VARIANT 3..5: note = X can be any amino acid
CGXXXCWLI

SEQ ID NO: 278 - protein, 7 aa, synthetic construct
  VARIANT 3: note = X can be any amino acid
  REGION >3: note = The amino acid 3 can be repeated three to seven times
CGXCWLI

SEQ ID NO: 279 - protein, 7 aa, synthetic construct
  REGION >3: note = The amino acid 3 can be repeated three to seven times
  VARIANT 3: note = X can be any amino acid
SGXSWLI

SEQ ID NO: 280 - protein, 6 aa, synthetic construct
RVLAEA

SEQ ID NO: 281 - protein, 7 aa, synthetic construct
ENLYFQS

SEQ ID NO: 282 - protein, 18 aa, synthetic construct
GNGNGNGNGNGNGNGNGN

SEQ ID NO: 283 - protein, 11 aa, synthetic construct
  MOD_RES 4: note = Dehydroalanine
  MOD_RES 8: note = Dehydroalanine
VGAXWLTXWVI

SEQ ID NO: 284 - protein, 5 aa, synthetic construct
  SITE 4: note = D-Tyrosine
  REGION 1..5: note = Peptide is circular
RGDYK

SEQ ID NO: 285 - protein, 5 aa, synthetic construct
TEVGA

SEQ ID NO: 286 - protein, 40 aa, synthetic construct
  MOD_RES 32: note = Dehydroalanine
SDLDIVDLDLAVDEELAALSVGGLGNTEVGAXGWLGSGVI

(19) United States
(12) Patent Application Publication
(10) Pub. No.: US 2026/0132172 A1
(43) Pub. Date: May 14, 2026

(54) IN VITRO BIOSYNTHESIS OF DIVERSE PYRIDINE-BASED MACROCYCLIC PEPTIDES

(71) Applicant: The Brd. of Trustees of the Univ. of Illinois, Urbana, IL (US)

(72) Inventors: Douglas Alan Mitchell, Brentwood, TN (US); Dinh Thanh Nguyen, Urbana, IL (US); Wilfred A. Van Der Donk, Champaign, IL (US)

(73) Assignee: The Brd. of Trustees of the Univ. of Illinois, Urbana, IL (US)

(21) Appl. No.: 18/874,931

(22) PCT Filed: Jun. 15, 2023
(86) PCT No.: PCT/US2023/068522

§ 371 Date: Dec. 13, 2024

Related U.S. Application Data
(60) Provisional application No. 63/352,345, filed on Jun. 15, 2022.
(60) Provisional application No. 63/442,530, filed on Feb. 1, 2023.
(60) Provisional application No. 63/455,974, filed on Mar. 30, 2023.

(51) Int. Cl. C07K 14/195; C12N 9/00; C12N 9/88

(52) U.S. Cl. CPC C07K 14/195; C12N 9/88; C12N 9/93; C12Y 402/01046

(57) ABSTRACT

Provided herein are compositions and methods for producing pyridine-based macrocyclic peptides.

### CROSS-REFERENCE TO RELATED APPLICATIONS

This application is a U.S. National Phase application under 35 U.S.C. § 371 of International Application PCT/US23/68522, filed on Jun. 15, 2023, which claims the benefit of U.S. Provisional Patent Application No. 63/352,345, filed Jun. 15, 2022, U.S. Provisional Patent Application No. 63/442,530, filed Feb. 1, 2023, and U.S. Provisional Patent Application No. 63/455,974, filed Mar. 30, 2023, where each application is incorporated by reference herein in their entireties.

### GOVERNMENT SUPPORT

This invention was made with Government support under 2R01GM097142 awarded by the National Institutes of Health. The Government has certain rights in this invention.

### BACKGROUND

Macrocyclic peptide natural products are a privileged class with many members exhibiting potent antibacterial, antifungal, antiviral, anticancer, and immunosuppressive activities. Compared to their linear counterparts, macrocyclic peptides possess desired properties, such as proteolytic stability, increased cell-membrane permeability, and conformational restrictions resulting in reduced entropy upon binding biological targets. These features have inspired interest in accessing macrocyclic peptides through combinatorial display, epitope-grafting, and cyclization of previously identified linear peptides with activity against biological targets. These efforts are greatly aided by versatile macrocyclization methods that tolerate a wide variety of peptide sequences and that can be executed with large-sized libraries.

Ribosomally synthesized and post-translationally modified peptides (RiPPs) routinely have macrocyclic structures. During RiPP biosynthesis, a gene-encoded precursor peptide undergoes modification by enzymes encoded in a biosynthetic gene cluster (BGC). RiPP precursor peptides are commonly composed of an N-terminal leader region responsible for recruiting biosynthetic proteins and a C-terminal core region that undergoes conversion to the mature RiPP. The physical separation of substrate binding from the site(s) of modification is an attractive feature of RiPP biosynthesis, as it facilitates access to a chemically diverse array of variants. Thus, libraries based on RiPP macrocyclic peptides have been constructed to yield analogs with reprogrammed bioactivity.

Thiopeptides are macrocyclic RiPPs associated with several enticing bioactivities of which potent inhibition of bacterial protein translation is the best studied. Structural analysis of thiopeptides reveals three universal functional groups: azole/azoline heterocycles derived from the ATP-dependent backbone cyclodehydration of Cys, Ser, and Thr residues; dehydroalanine/dehydrobutyrine (Dha/Dhb) and their derivatives resulting from the glutamylation and subsequent elimination of Ser and Thr residues; and a class-defining, six-membered nitrogenous heterocycle resulting from a formal [4+2]-cycloaddition of two Dha-like residues that coincides with elimination of water and the leader peptide. Accessing thiopeptide derivatives beyond single amino acid substitutions has been challenging because of the requirement of multiple azoles in the peptide for downstream Dha formation and [4+2] cycloaddition. The only thiopeptide thus far shown to be amenable to multi-site variation is lactazole, for which macrocyclization requires only two azoles and three Dha residues.

A minimalistic, thiopeptide-like BGC from Micromonospora rosaria that encodes two precursor peptides without Cys residues. The BGC also lacks the genes for azol(in)e formation and was predicted to produce a pyridine-based macrocyclic peptide (i.e., pyritide, FIG. 1). Methods are needed in the art to produce variant macrocyclic peptides.

### SUMMARY

An aspect provides a substrate for enzyme synthesis of pyridine-based macrocyclic peptides comprising a leader region and a core region, wherein the leader region comprises;
X₁LDX₂X₃X₄X₅X₆LX7X₈X₉X₁₀X₁₁LX₁₂X₁₃X₁₄X₁₅X₁₆X₁₇GLGNTEVGA (SEQ ID NO:1),
wherein
X₁ is D, S or A;
X₂ is I or V;
X₃ is V, T, M, or A;
X₄ is D, N, or T;
X₅ is L or V;
X₆ is D or E;
X₇ is A or P;
X₈ is V, I, or G;
X₉ is D, E, or S;
X₁₀ is E or D;
X₁₁ is E, L, V, or absent;
X₁₂ is A or V;
X₁₃ is A, E, or K;
X₁₄- is L, V, or A;
X₁₅ is S, L, or V;
X₁₆ is V, I, G, T, or A;
X₁₇ is G or M; and
wherein the core region comprises:
SGX₁SX₄X₂X₃ (SEQ ID NO:10),
   wherein X₁ is three to twenty amino acids, and wherein X₂ is V or L, wherein X₃ is I or V, wherein X₄ is Y, W, F, or H, and wherein the leader and core can be separate polypeptides used in combination, a single fusion protein, or covalently linked polypeptides.

The leader region can comprise: DLDIVX₁LDLX₂X₃DEELAAX₄SVGGLGNTEVGA (SEQ ID NO:2),
wherein;
X₁ is D, N, or T;
X₂ is A or P;
X₃ is V, I, or G; and
X₄ is L, V, or A.

The leader region can also comprise:

(SEQ ID NO: 3)
DLDIVDLDLAVDEELAALSVGGLGNTEVGA
(SEQ ID NO: 4)
DLDIVNLDLPIDEELAAVSVGGLGNTEVGA
(SEQ ID NO: 5)
DLDIVDLDLPIDEELAAVSIGGLGNTEVGA
(SEQ ID NO: 6)
SLDVTTVELPGED LVEALGMGLGNTEVGA
(SEQ ID NO: 7)
SLDVMTVELPGED LVKALGMGLGNTEVGA
(SEQ ID NO: 8)
SLDVATVELPGSDLLVEAVTMGLGNTEVGA
(SEQ ID NO: 9)
ALDVATVELPGSEVLVEAVAMGLGNTEIGA

A core region can comprise:

(SEQ ID NO: 11)
SGX₁SX₃X2I,

wherein X₁ is three to 100 amino acids and wherein the last of the three to 100 amino acids is a positively charged amino acid, and wherein X₂ is V or L, and wherein X₃ is Y, W, F, or H. The core region can comprise: SGFFX₁SWX₂I (SEQ ID NO:12), wherein X₁ is three to 100 amino acids, wherein X₂ is V or L, and wherein X₃ is Y, W, F, or H.

A substrate can further comprise a linker region and a handle region at the C-terminus of the core region. The handle region can be for amplification, detection, or purification. The handle region can comprise a polypeptide or nucleic acid molecule for yeast display, phage display, mRNA display, TRAP display, or ribosome display. The linker can be a flexible linker, a cleavable linker, or a rigid linker.

Another aspect provides a fusion protein comprising:
(a) Micromonospora dehydratase (MroB or MroC or both MroB and MroC) and an affinity tag; or
(b) Micromonospora macrocyclase (MroD) and an affinity tag.

The affinity tag can be a polyhistidine (poly-His) tag, a hemagglutinin (HA) tag, an AviTag protein C tag, a FLAG tag, a Strep-tag II, aTwin-Strep-tag, a glutathione-S-transferase (GST) tag, a C-myc tag, a chitin-binding domain, a streptavidin binding protein (SBP), a maltose binding protein (MBP), a cellulose-binding domain, a calmodulin-binding peptide, or an S-tag. The fusion protein can further comprise a linker.

Yet another aspect provides a method of making a pyridine-based macrocyclic peptide comprising contacting the substrate for enzyme synthesis of pyridine-based macrocyclic peptides as described herein with MroB, MroC, and MroD. The MroB, MroC, and MroD can be fused to an affinity tag. Rings with 14 to 23 members can be made.

Even another aspect provides a substrate for enzyme synthesis of pyridine-based macrocyclic peptides comprising a leader sequence of:
MDNVVTEAAEFADLDIDDFDLAVDEELAALSVGGLGNTEVGA (SEQ ID NO: 36), and core sequence can be SCNCFCYICCSX₁LI (SEQ ID NO:37), wherein X₁ is Y, W, F, or H, or SCX₂CX₂CX₂ICCSX₁LI (SEQ ID NO:43), wherein X₁ is Y, W, F, or H, and wherein X₂ is any amino acid; or
MDNVVTEAAEFADLDIDDFDLAVDEELAALSVGGLGNTEVGA (SEQ ID NO: 36) and a core sequence of SXIN XIF X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof or a core sequence of SX₁X₃ X₁X₃ X₁X₃₁ X₁ X₁SX₂LI, (SEQ ID NO:44) wherein X₂ is Y, W, F, or H, wherein each of X₁ are thiazole, thiazoline, oxazole, oxazoline, methyloxazole, methyloxazoline, or combinations thereof, and wherein X₃ is any amino acid, and wherein the leader and core can be separate polypeptides used in combination, a single fusion protein, or covalently linked polypeptides.

The substrate can further comprise a linker region and a handle region at the C-terminus of the core region.

Even another aspect provides a method of making pyridine-based macrocyclic peptides comprising using a first substrate, wherein the first substrate comprises: a leader region of MDNVVTEAAEFADLDIDDFDLAVDEELAALSVGGLGNTEVGA (SEQ ID NO:36) and a core sequence of SCNCFCYICCSXILI (SEQ ID NO:37), wherein X₁ is Y, W, F, or H or SCX₂CX₂CX₂ICCSX₁LI (SEQ ID NO:43), wherein X₁ is Y, W, F, or H, and wherein X₂ is any amino acid. The first substrate is contacted with thiazole synthetase, TbtE, TbtF, TbtG, or TbtD such that a second substrate is formed as follows:
a leader region of MDNVVTEAAEFADLDIDDFDLAVDEELAALSVGGLGNTEVGA (SEQ ID NO: 36) and a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof, or a core sequence of SX₁X₃ X₁X₃ X₁X₃I X₁ X₁SX₂LI, (SEQ ID NO:44) wherein X₂ is Y, W, F, or H, wherein each of X₁ are thiazole, thiazoline, oxazole, oxazoline, methyloxazole, methyloxazoline, or combinations thereof, and wherein X₃ is any amino acid.

The leader region and core can be separate polypeptides used in combination, a single fusion protein, or covalently linked polypeptides. The second substrate is contacted with MroB, MroC, and MroD to form pyridine-based macrocyclic peptides. The first substrate, the second substrate, or both the first and second substrates can further comprise a linker region and a handle region at the C-terminus of the core region. The MroB, MroC, and MroD can be fused to an affinity tag.

Yet another aspect provides a method of making pyridine-based macrocyclic peptides with a substrate comprising a leader region of:
MDNVVTEAAEFADLDIDDFDLAVDEELAALSVGGLGNTEVGA (SEQ ID NO: 36) and a core sequence of SX₁N X₁F X₁YI X₁ X₁SX₂LI (SEQ ID NO:38), wherein X₂ is Y, W, F, or H, wherein each of X₁ are thiazole, thiazoline, oxazole, oxazoline, methyloxazole, methyloxazoline, or combinations thereof, or a core sequence of SX₁X₃ X₁X₃ X₁X₃I X₁X₁SX₂LI (SEQ ID NO:44), wherein X₂ is Y, W, F, or H, wherein each of X₁ are thiazole, thiazoline, oxazole, oxazoline, methyloxazole, methyloxazoline, or combinations thereof, and wherein X₃ is any amino acid. The leader region and core can be separate polypeptides used in combination, a single fusion protein, or covalently linked polypeptides. The substrate can be contacted with MroB, MroC, and MroD to form pyridine-based macrocyclic peptides. The substrate can further comprises a linker region and a handle region at the C-terminus of the core region. The MroB, MroC, and MroD can be fused to an affinity tag. The pyridine-based macrocyclic peptides can comprise one or more thiazole, thiazoline, oxazole, oxazoline, methyloxazole, or methyloxazoline groups.

Another aspect provides a method of making a pyridine-based macrocyclic peptide. The method comprises contacting a first substrate comprising:
VESLTAGHGMTEVGADhaX1 (SEQ ID NO:41), wherein X₁ is thiazole, thiazoline, oxazole, oxazoline, methyloxazole, or methyloxazoline; and a second substrate comprising: Ac-VX₁X₂DhaX₃Dha (SEQ ID NO:42), wherein X₁ and X₂ and X₃ are thiazole, thiazoline, oxazole, oxazoline, methyloxazole, methyloxazoline, or a combination thereof, with one or more polypeptides comprising 90% or more sequence identity to TbtE, TbtF, TbtG, or TbtD, such that a pyridine-based macrocyclic peptide is made.

Another aspect provides a substrate for enzyme synthesis of pyridine-based macrocyclic peptides comprising a leader and core sequence where the macrocyclic peptides can comprise various permutations of RGD in the core sequence. In some aspects, the core region comprises:

(SEQ ID NO: 45)
SGX₀₋₃RGDX₀₋₃SWLI
or
(SEQ ID NO: 46)
CGX₀₋₃RGDX₀₋₃CWLI.

wherein X is any of the 20 proteinogenic amino acids.

The core sequence can be:

(SEQ ID NO: 47)
CGRGDRCWLI
(SEQ ID NO: 48)
CGFRGDAGCWLI
(SEQ ID NO: 49)
CGRGDFVGCWLI
(SEQ ID NO: 50)
CGRGDFVAGCWLI
(SEQ ID NO: 51)
SGRGDRSWLI
(SEQ ID NO: 52)
SGFRGDAGSCWLI
(SEQ ID NO: 53)
SGRGDFVGSWLI
(SEQ ID NO: 54)
SGRGDFVAGSWLI

The leader sequence that can be used with the RGD in the core can be any suitable leader sequence described herein. In some aspects, the leader sequence comprises:

(SEQ ID NO: 1)
X₁LDX₂X₃X₄X₅X₆LX₇X₈X₉X₁₀X₁₁LX₁₂
X₁₃X14X₁₅X₁₆X₁₇GLGNTEVGA,

wherein
X₁ is D, S or A;
X₂ is I or V;
X₃ is V, T, M, or A;
X₄ is D, N, or T;
X₅ is L or V;
X₆ is D or E;
X₇ is A or P;
X₈ is V, I, or G;
X₉ is D, E, or S;
X₁₀ is E or D;
X₁₁ is E, L, V, or absent;
X₁₂ is A or V;
X₁₃ is A, E, or K;
X₁₄- is L, V, or A;
X₁₅ is S, L, or V;
X₁₆ is V, I, G, T, or A;
X₁₇ is G or M;

A method of making a pyridine-based macrocyclic peptide comprising contacting the substrate for enzyme synthesis of pyridine-based macrocyclic peptides with the RGD core. A method of making pyridine-based macrocyclic peptides comprising:

(SEQ ID NO: 45)
SGX₀₋₃RGDX₀₋₃SWLI
or
(SEQ ID NO: 46)
CGX₀₋₃RGDX₀₋₃CWLI.

a leader region and
   a core sequence of SGX₀₋₃RGDX₀₋₃SWLI (SEQ ID NO:45) or CGX₀₋₃RGDX₀₋₃CWLI (SEQ ID NO:46) wherein X₃ is any amino acid,
with MroB, MroC, and MroD, wherein the leader region and core sequence can be separate polypeptides used in combination, a single fusion protein, or covalently linked polypeptides, such that pyridine-based macrocyclic peptides are made with MroB, MroC, or MroD, and wherein the leader sequence that can be used with the RGD in the core can be any suitable leader sequence or any leader sequence described herein.

### BRIEF DESCRIPTION OF THE DRAWINGS

FIG. 1. Biosynthesis of pyritides. (A) BGC from Micromonospora rosaria and sequences of precursor peptides. (B) Reactions catalyzed by MroB and MroC. (C) Reaction catalyzed by the [4+2] macrocyclase MroD. (D) Structure of pyritide A1 with the class-defining pyridine.

FIG. 2. Substrate scope of MroBCD. Unless otherwise stated, all peaks represent [M+H]⁺. (A) Summary of results from assays in which MroA2 variants reacted with MroBCD (B) Representative MALDI-TOF-MS of MroA2 variants at Phe3, Phe4, Gly5, and Arg6 processed by MroBCD. (C) Macrocycle formation from substrates with conservative substitutions of Gly2, Trp8, Leu9, and Ile10

FIG. 3. Panel of variant pyritides. Variations were made in regions in bold. (A) MALDI-TOF-MS of representative multi-site pyritide variants. (B) MALDI-TOF-MS of a 68-membered pyritide macrocycle through substitution of Gly by (GlyAsn) 9. (C) LC-HR-ESI-MS of a pyritide containing four thiazoles and one thiazoline. Thiazol(in)e residues are bolded and abbreviated as Thz.

FIG. 4. LC-ESI-MS/MS of MroA1 variants treated with MroBC. (A) Product obtained with Δ12MroA1-W7G, showing Ser1 was dehydrated. (B) Product obtained with GlyAla-MroA1 core, showing Ser6 was dehydrated.

FIG. 5. Sequence alignment of M. rosaria tRNAGlu (CUC) and T. bispora tRNAGlu (CUC). The sequence identity is 91%.

FIG. 6. MALDI-TOF-MS analysis of Arg variants of uncyclized precursor peptides after MroB/C/D treatment. The sequences of original uncyclized precursors and their corresponding Arg variants are indicated in each mass spectrum. The sequence of MroA2 precursor peptide with the varied region in bold is shown. All spectra were acquired using reflector positive mode of MALDI-TOF-MS. Unless otherwise stated, all peaks are [M+H]+. The precursor peptides were generated through in vitro translation (see Experimental Methods). The f in the precursor peptide sequence represents a formyl group, which results from formyl-methionine utilized in in vitro translation. The pyritide macrocycles, the ejected leader peptides, and the remaining didehydrated intermediates are annotated accordingly.

FIG. 7. Sequence alignment of pyritide precursor peptides. The sequences were identified from the GenBank database and aligned according to a previously reported bioinformatic protocol.² The table shows all identified pyritide precursor peptides found up to May 2022. The NCBI accessions of the precursor peptides are shown on the left. The box captures the amino acid residues removed to generate Δ12MroA1, which was utilized in fluorescence polarization experiments. The start codon of the last two precursor peptides were potentially misidentified by GenBank.

FIG. 8. LC-HR-ESI-MS/MS analysis of a 32-membered macrocycle produced by MroB/C/D. The product was generated through MroB/C/D reaction with substrate synthesized in a 15 μL scale in vitro translation.

FIG. 9. LC-HR-ESI-MS/MS analysis of a 38-membered macrocycle produced by MroB/C/D. The product was generated through MroB/C/D assays with substrate synthesized in a 15 μL scale in vitro translation.

FIG. 10. LC-HR-ESI-MS/MS analysis of a 62-membered macrocycle produced by MroB/C/D. The product was generated through MroB/C/D assays with substrate synthesized in a 15 μL scale in vitro translation.

FIG. 11. LC-HR-ESI-MS/MS analysis of a 68-membered macrocycle produced by MroB/C/D. The product was generated through MroB/C/D assays with substrate synthesized in a 15 μL scale in vitro translation.

FIG. 12. Large macrocycle sizes produced by MroB/C/D. All results were acquired using reflector positive mode MALDI-TOF-MS. The crystallization matrix utilized in this experiment was Super DHB. Unless otherwise stated, all peaks are [M+H]+. The precursor peptides were generated through in vitro translation (see Experimental Methods). The f in the precursor peptide sequence represents a formyl group, which results from formyl-methionine utilized in in vitro translation. The pyritide macrocycles and the ejected leader peptides are annotated accordingly.

FIG. 13. MroB/C/D produces 62-membered macrocycles with different sequences. The core sequences are indicated next to each mass spectrum. The different region between each sequence is in bold in the full-length precursor peptide (X=random amino acids except Cys, Ser, Thr). All results were acquired using reflector positive mode MALDI-TOF-MS. Unless otherwise stated, all peaks are [M+H]+. The precursor peptides were generated through in vitro translation (see Experimental Methods). The f in the precursor peptide sequence represents a formyl group, which results from formyl-methionine utilized in in vitro translation. The pyritide macrocycles, the ejected leader peptides, the didehydrated intermediates, and the monodehydrated intermediates are annotated accordingly.

FIG. 14. MroBCD produce a pyritide containing thiazol(in)es. (A) Incorporation of critical residues for thiazole-forming enzymes TbtE/F/G to MroA1 leader peptide and the design of the substrate processed by TbtE/F/G and MroB/C/D. Residues critical to TbtE/F/G activity are bolded. 16 Cys residues undergoing cyclodehydration by TbtEFG are bolded, while Ser residues undergoing dehydration by MroBC are bolded. (B) Mass spectral analysis of thiazol(in)e formation, dehydration, and cyclization. The sequence of the utilized precursor peptide is shown (1). The f in the precursor peptide sequence represents a formyl group, which results from formyl-methionine utilized in in vitro translation. All spectra were acquired by reflector positive mode MALDI-TOF-MS. Unless otherwise stated, all peaks are [M+H]+. The top MALDI-TOF mass spectrum shows the unmodified precursor peptide, which underwent five carbamidomethylations after treating with iodoacetamide (IAA). The third spectrum shows that five Cys residues were converted to four thiazoles and one thiazoline after treating with Tbt/E/F/G. This intermediate did not undergo carbamidomethylation after adding IAA (2). The fifth and sixth spectrum demonstrate that the precursor containing thiazol(in)e residues underwent two dehydrations by MroB/C followed by [4+2] cyclization by MroD. The last four spectra are different mass regions of 2 and 2+MroD. CAM=carbamidomethyl.

FIG. 15. Sequence of Mro biosynthetic genes and T. bispora GluRS for optimal E. coli expression. All sequences are provided 5′ to 3′. Restriction sites for cloning are underlined (5′ BamHI, 3′ XhoI). These gene constructs were synthesized by GenScript (Piscataway, NJ, USA).

FIG. 16. Nucleotide sequence of open reading frames inserted into plasmids used in this study.

FIG. 17. RDG epitope grafting. A, shown are MALDI-ToF-MS spectra of various MroA variants with both pyridine-forming serines replaced with cysteine. The “Gly2” position of the ring is retained, as well as the native WLI tail. In bold are the non-native motifs containing the grafted integrin epitopes. B, MALDI-ToF-MS spectrum of FITC-labeled cyclic RGDyK, where the lowercase y indicates D-Tyr. C, Molecular structures that correspond with the MS data.

FIG. 18. TRAP display of pyritides to determine substrate tolerance. Top, a construct is prepared that encodes a library of pyritide precursor peptides featuring the following: N-terminal biotinylation of the leader peptide, replacement of the pyridine-forming serines with cysteine, a variable region between the two cysteines, the WLI tail, and the HA epitope tag. Following the TRAP display procedure, C-terminus is linked to the encoding DNA by puromycin (Puro). After treatment chemical dehydrothiolation, the didehydrated peptide library will be subjected to MroD treatment. Tolerated sequences (i.e., substrates) will form mature pyritide-nucleic acid conjugates with the biotinylated leader peptide eliminated. Non-substrate sequences will retain the biotinylated leader, thus allowing for facile discrimination between of substrates and non-substrates by NovaSeq on the flow-thru and elutants of a streptavidin-based separation, respectively.

FIG. 19. TRAP display to evolve integrin-binding pyritides. A, shown is a TRAP workflow with use of 5-fluorophore labeled oligonucleotide that is complementary to the mRNA encoding for the pyritide. Treatment with the MroD yields mature pyritide-TRAP-fluorophore conjugates while immobilized streptavidin is used to remove non-substrates and the excised leader peptide. The non-biotinylated fraction (desired product) is allowed to bind to the target of interest (TOI) in reconstituted liposomes. FACS collects the fluorescent liposomes which separates binders from non-binders. Post-FACS NovaSeq runs identify binders and non-binders, while PCR amplifies binders for the next round (if desired). Implicit to this design is the ease of alteration to employ MroB/C and a di-serine containing substrate peptide in place of chemical dehydrothiolation. B, shown is an alternative plan that avoids the use of fluorescent labels, liposomes, and FACS. The workflow deviates from panel A in that the TOI is biotinylated and magnetic Dynabeads are used to separate binders from non-binders. We will evaluate which method is superior in this proof-of-concept project.

FIG. 20. Chemical dehydrothiolation to “bypass” MroB/C. The current chemoenzymatic route to obtain pyritide A1 and variants thereof. First, the two serine residues that comprise the pyridine are substituted with cysteine. Upon reaction with methyl 2,5-dibromopentoate, the didehydrated substrate of MroD is obtained.

FIG. 21. Pyritide natural variation. Our bioinformatics software RODEO has identified several dozen bacterial species that encode pyritide BGCs. All unique precursor peptide sequences found are displayed. The invariant GA comprises the end of the leader region while the most N-terminal Ser (bold) represents the start of the core region. The two bold Ser are incorporated into the pyridine, giving rise to natural macrocycles sizes that range from 5-7 residues.

### DETAILED DESCRIPTION

Macrocyclic peptides are sought-after molecular scaffolds for drug discovery and new methods to access diverse libraries are of increased interest. Here, we report the enzymatic synthesis of pyridine-based macrocyclic peptides (pyritides) from linear precursor peptides. Pyritides are a recently described class of ribosomally synthesized and post-translationally modified peptides (RiPPs) and are related to the long-known thiopeptide natural products. RiPP precursors typically contain an N-terminal leader region that is physically engaged by the biosynthetic proteins that catalyze modification of the C-terminal core region of the precursor peptide. We demonstrate that pyritide-forming enzymes recognize both the leader region and a C-terminal tripeptide motif, with each contributing to site-selective substrate modification. Substitutions in the core region were well-tolerated and facilitated the generation of a wide range of pyritide analogs, with variations in macrocycle sequence and size. A combination of the pyritide biosynthetic pathway with azole-forming enzymes are utilized herein to generate a thiazole-containing pyritide (historically known as a thiopeptide) with no similarity in sequence and macrocycle size to the naturally encoded pyritides. The broad substrate scope of the pyritide biosynthetic enzymes serves as a platform for macrocyclic peptide lead discovery and optimization.

Reasoning that the absence of thiazol(in)es would render the pyritide biosynthetic pathway more tolerant of substitutions in the core region, the substrate selectivity of pyritide biosynthesis was used to identify macrocycle-forming biosynthetic enzymes with broad substrate tolerance.

### Substrates for Synthesis of Pyridine-Based Macrocyclic Peptides

Substrates for enzyme synthesis of pyridine-based macrocyclic peptides can comprise a leader region, a core region and, optionally, a linker and/or handle region. In an aspect a substrate for enzyme synthesis of pyridine-based macrocyclic peptides can comprise a leader region wherein the leader region comprises:

(SEQ ID NO: 1)
X₁LDX₂X₃X₄X₅X₆LX₇X₈X₉X₁₀X₁₁LX₁₂
X₁₃X14X₁₅X₁₆X₁₇GLGNTEVGA,

wherein X₁ is D, S or A; X₂ is I or V; X₃ is V, T, M, or A; X₄ is D, N, or T; X₅ is L or V; X₆ is D or E; X₇ is A or P; X₈ is V, I, or G; X₉ is D, E, or S; X₁₀ is E or D; X₁₁ is E, L, V, or absent; X₁₂ is A or V; X₁₃ is A, E, or K; X₁₄ is L, V, or A; X₁₅ is S, L, or V; X₁₆ is V, I, G, T, or A; X₁₇ is G or M.

In an aspect a leader region comprises:

DLDIVX₁LDLX₂X₃DEELAAX₄SVGGLGNTEVGA (SEQ ID NO:2), wherein: X₁ is D, N, or T; X₂ is A or P; X₃ is V, I, or G; and X₄ is L, V, or A.

In an aspect a leader region comprises:

(SEQ ID NO: 3)
DLDIVDLDLAVDEELAALSVGGLGNTEVGA
(SEQ ID NO: 4)
DLDIVNLDLPIDEELAAVSVGGLGNTEVGA
(SEQ ID NO: 5)
DLDIVDLDLPIDEELAAVSIGGLGNTEVGA
(SEQ ID NO: 6)
SLDVTTVELPGEDLVEALGMGLGNTEVGA
(SEQ ID NO: 7)
SLDVMTVELPGEDLVKALGMGLGNTEVGA
(SEQ ID NO: 8)
SLDVATVELPGSDLLVEAVTMGLGNTEVGA
(SEQ ID NO: 9)
ALDVATVELPGSEVLVEAVAMGLGNTEIGA

A core region can comprise: SGX₁SWX₂X₃ (SEQ ID NO:10), wherein X₁ is three to 100 (e.g. 3, 5, 10, 15, 20, 30, 40, 50, 60, 70, 80, 90, 100 or more) amino acids, and wherein X₂ is V or L, wherein X₃ is I or V. The last of the three to 100 amino acids can be a positively charged amino acid. A core region can comprise SGX₁SX₃X₂I (SEQ ID NO:11), wherein X₁ is three to 100 (e.g., 3, 5, 10, 15, 20, 30, 40, 50, 60, 70, 80, 90, 100 or more) amino acids, wherein X₃ is Y, W, F, or H, and wherein X₂ is V or L. The last of the three to 100 amino acids can be a positively charged amino acid. A core region can comprise SGFFX₁SWX₂I (SEQ ID NO:12), wherein X₁ is three to 100 (e.g., 3, 5, 10, 15, 20, 30, 40, 50, 60, 70, 80, 90, 100 or more) amino acids, and wherein X₂ is V or L. The last of the three to 100 amino acids can be a positively charged amino acid. Positively charged amino acids include: H, K, and R.

A leader and a core can be separate polypeptides used in combination as a single fusion protein, or covalently linked polypeptides.

Other substrates that can be used for making for enzyme synthesis of pyridine-based macrocyclic peptides can comprise a leader of:
MDNVVTEAAEFADLDIDDFDLAVDEELAALSVGGLGNTEVGA (SEQ ID NO: 36). A core sequence can be SCNCFCYICCSX₁LI (SEQ ID NO:37), wherein X₁ is Y, W, F, or H. A core sequence can also be SCX₂CX₂CX₂ICCSX₁LI (SEQ ID NO:43), wherein X₁ is Y, W, F, or H, and wherein X₂ is any amino acid. This substrate can be reacted with the heterocycle synthetase TbtE, TbtF, TbtG, TbtD, or combinations thereof, or homologous proteins thereof (e.g., all proteins discussed in the “Thiazole Synthetases” section below) to result in a leader comprising:
MDNVVTEAAEFADLDIDDFDLAVDEELAALSVGGLGNTEVGA (SEQ ID NO: 36) and a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof or a core sequence of SX₁X₃ X₁X₃ X₁X₃I X₁ X₁SX₂LI, (SEQ ID NO:44) wherein X₂ is Y, W, F, or H, wherein each of X₁ are thiazole, thiazoline, oxazole, oxazoline, methyloxazole, methyloxazoline, or combinations thereof, and wherein X₃ is any amino acid.

The substrate can then be reacted with any of MroB, MroC, and/or MroD as described in detail below. These substrates (leader and core) can additionally comprise a linker and/or handle region as described below.

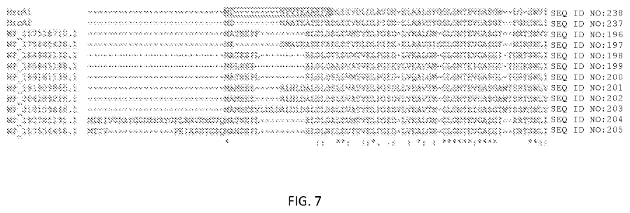

The genome-mining tool known as Rapid ORF Description and Evaluation Online (RODEO) can be used to identify biosynthetic gene clusters based on available genomic information for other RiPP classes including pyritides (and the specialized pyritides formerly known as thiopeptides). We identified that nature encodes a high level of variability in some portions of the pyritide precursor (FIG. 7). Shown in FIG. 7 are all currently identified sequences (redundancy removed). Key regions of note are the conserved DL and LD segments of the leader region, which serve as the recognition sequence for the RiPP precursor

Recognition Element (RRE) domain that comprises one domain of MroB homologs. Also conserved is the most C-terminal portion of the leader, often “TEVGA” SEQ ID NO:285, the two serines comprising the pyridine (with spacing ranging from 5-7 residues), and the most C-terminal portion of the core, often “WLI” or “WLV”. The natural variation within the pyritide macrocycle bodes well for “pyritide display”, as it can be expanded, contracted, and tolerates a wide spectrum of amino acids at most locations.

Other substrates that can be used for making for enzyme synthesis of pyridine-based macrocyclic peptides can comprise various permutations of RGD in the core region. In some aspects the core region comprises:
SGX₀₋₃RGDX₀₋₃SWLI (SEQ ID NO:45) or CGX₀₋₃RGDX₀₋₃CWLI (SEQ ID NO: 46), wherein is X is any of the 20 proteinogenic amino acids.

In other aspects, the core sequence can be:

(SEQ ID NO: 47)
CGRGDRCWLI
(SEQ ID NO: 48)
CGFRGDAGCWLI
(SEQ ID NO: 49)
CGRGDFVGCWLI
(SEQ ID NO: 50)
CGRGDFVAGCWLI
(SEQ ID NO: 51)
SGRGDRSWLI
(SEQ ID NO: 52)
SGFRGDAGSCWLI
(SEQ ID NO: 53)
SGRGDFVGSWLI
(SEQ ID NO: 54)
SGRGDFVAGSWLI.

In some aspects, the RGD core described herein includes any suitable leader sequence or any leader sequence or region described herein.

### Linker and Handle Region

A substrate can further comprise a linker region and a handle region at the C-terminus of the core region. The handle region can be for amplification, detection, or purification. The handle region can comprise a tag, such as an affinity tag, or a detector molecule such as a fluorescent protein, a poly His tag, a GST tag, an epitope tag, a FLAG tag, or a chemical dye. The handle region can comprise a polypeptide or nucleic acid molecule for yeast display, phage display, mRNA display, TRAP display, or ribosome display.

In an example, a handle region can comprise, e.g., Agα1p, Aga2p, Cwp1p, Cwp2p, Tip1p, Flo1p, Sed1p, YCR89w, and Tir1 for yeast display (see Kondo A, Ueda M. Yeast cell-surface display—applications of molecular display. Appl Microbiol Biotechnol. 2004:64:28-40; Cherf G M, Cochran J R. Applications of Yeast Surface Display for Protein Engineering. Methods Mol Biol. 2015:1319:155-75), a phage coat protein (e.g., p3, p6, p7, p8 and p9) for phage display (see Velappan et al., A comprehensive analysis of filamentous phage display vectors for cytoplasmic proteins: an analysis with different fluorescent proteins. Nucleic Acids Res. 2010 March: 38 (4):e22), a covalent bond between a protein and its encoding mRNA via a small molecule puromycin linker for mRNA display. mRNA templates used for mRNA display technology have puromycin (or variant of puromycin) at their 3′ end such that as translation proceeds, the ribosome moves along the mRNA template, and once it reaches the 3′ end of the template, the fused puromycin will enter the ribosome's A site and be incorporated into the nascent peptide, the mRNA-polypeptide fusion is then released from the ribosome. A handle region can be for TRAP display (transcription-translation coupled with association of puromycin linker), which automatically produces a polypeptide library through a series of sequential reactions: transcription, association of puromycin-DNA linker, translation, and conjugation between the nascent polypeptide and puromycin-DNA linker (or variant thereof) (see Ishizawa et al., J. Am. Chem. Soc. 2013, 135, 14, 5433-5440 (2013). This attachment is non-covalent and uses hybridization of two nucleic acids to retain a phenotype-genotype linkage.

### MroB, MroC, and MroD

In some aspects and methods MroB (Micromonospora dehydratase), MroC (Micromonospora dehydratase), and MroD (Micromonospora macrocyclase) enzymes are provided.

A MroB polypeptide can be a MroB polypeptide from Micromonospora rosaria (NCBI accession WP_067368389.1), M. yangpuensis (NCBI accession WP_091433993.1; WP_229688411.1, GGM10370.1), or any other suitable MroB polypeptide. In an aspect, a MroB polypeptide comprises 80, 85, 90, 95, 96, 97, 98, 99% or more sequence identity to Micromonospora rosaria (NCBI accession WP_067368389.1) or M. yangpuensis (NCBI accession WP_091433993.1; WP_229688411.1, GGM10370.1).

A MroC polypeptide can be a MroC polypeptide from Micromonospora rosaria (NCBI accession WP_083978639.1), M. yangpuensis (NCBI accession WP_175440427), or any other suitable MroC polypeptide. In an aspect, a MroC polypeptide comprises 80, 85, 90, 95, 96, 97, 98, 99% or more sequence identity to Micromonospora rosaria (NCBI accession WP_083978639.1) or M. yangpuensis (NCBI accession WP_175440427).

A MroD polypeptide can be a MroD polypeptide from Micromonospora rosaria (NCBI accession WP_067368384.1), Micromonospora fluostatini (NCBI accession TDC02021.1), Micromonospora yangpuensis (NCBI accession WP_091433994.1), or any other suitable MroD polypeptide. In an aspect, a MroD polypeptide comprises 80, 85, 90, 95, 96, 97, 98, 99% or more sequence identity to a MroD polypeptide from Micromonospora rosaria (NCBI accession WP_067368384.1), Micromonospora fluostatini (NCBI accession TDC02021.1) or Micromonospora yangpuensis (NCBI accession WP_091433994.1),

### MroB, MroC, and MroD Fusion Proteins

In some aspects, MroB, MroC, and/or MroD can be present in a fusion protein. A fusion protein can comprise Micromonospora dehydratase (MroB or MroC or both MroB and MroC) and a tag such as an affinity tag and/or Micromonospora macrocyclase (MroD) and a tag, such as an affinity tag.

A tag can be, for example, a polyhistidine (poly-His) tag, a hemagglutinin (HA) tag, an AviTag protein C tag, a FLAG tag, a Strep-tag II, aTwin-Strep-tag, a glutathione-S-transferase (GST) tag, a C-myc tag, a chitin-binding domain, a streptavidin binding protein (SBP), a maltose binding protein (MBP), a cellulose-binding domain, a calmodulin-binding peptide, or an S-tag. A tag can be present at the amino or carboxy terminus of an MroB, MroC, or MroD protein.

A fusion protein can further comprise a linker. For example, a linker can occur between an Mro protein and an affinity tag. In another aspect, a linker can occur at any position in the fusion protein (at the amino or carboxy terminus).

In an aspect, MroB, MroC, and/or MroD (with or without fusion to a tag, such as an affinity tag) can be co-expressed with Thermobispora bispora GluRS ((NCBI accession ADG89504.1) and T. bispora tRNAGlu (CUC) or M. rosaria tRNAGlu (CUC), which share 91% sequence identity. (FIG. 5). In an aspect a GluRS polypeptide can comprise about 70, 80, 85, 90, 95, 96, 97, 98, 99%, or more sequence identity to NCBI accession ADG89504.1. In an aspect a polynucleotide comprises about 70, 80, 85, 90, 95, 96, 97, 98, 99%, or more sequence identity to T. bispora tRNAGlu (CUC) or M. rosaria tRNAGlu (CUC).

### Linkers

As described above, a linker can be present in a substrate (e.g., a substrate can comprise a leader, a core, a linker, and a handle region). A linker can also be present in an MroB, MroC, and/or MroD fusion protein. In some aspects, substrates that comprise a linker and handle, each of the linker and handle are individual elements and can be the same or different elements. In some aspects, substrates that comprise a linker, a handle, and a detector molecule, each of the linker, the handle, and the detector molecule are individual elements and can be the same or different elements. In some aspects, a MroB, MroC, or MroD that comprise a linker and a tag, each of the linker and the tag are individual elements and can be the same or different elements.

A linker can be any suitable linker including, e.g., flexible linkers, rigid linkers, and cleavable linkers. A linker can be a random sequence, e.g., Gly-Ser repeats of varying lengths, an epitope, or affinity tag (e.g., HA, c-myc. FLAG. His-tag, etc.), proteolytic motif (e.g., TEVp. EK, factor Xa, thrombin, precision protease, etc.). Besides the basic role in linking the functional domains together (as in flexible and rigid linkers) or releasing free functional domain in vivo or in vitro (as in cleavable linkers), linkers can improve biological activity, increase expression yield, and provide desirable pharmacokinetic profiles. A linker can be a linker that can increase or stability of the fusion protein or improve protein folding (e.g., scFv (a flexible linker: (GGGGS); (SEQ ID NO:13)), Myc-Est2p (a flexible linker: (Gly)₈) (SEQ ID NO: 14)), albumin-ANF (a flexible linker: (Gly)₆) (SEQ ID NO:15)), virus coat protein (a rigid linker; (EAAAK)₃; (SEQ ID NO:16)), beta-glucanase-xylanase (a rigid linker; (EAAAK)ₙ (n=1-3) (SEQ ID NO:17)). A linker can be a linker that can increase protein expression (e.g., hGH-Tf and Tf-hGH, a rigid linker, A (EAAAK)₄ALEA (EAAAK)₄A (SEQ ID NO:18)), G-CSF-Tf and Tf-G-CSF (a rigid linker, A (EAAAK)₄ALEA (EAAAK)₄A (SEQ ID NO:19)), G-CSF-Tf (flexible linker, (GGGGS)₃, SEQ ID NO:20)), G-CSF-Tf (rigid linker, A(EAAAK)₄ALEA(EAAAK)₄A (SEQ ID NO:21)), HSA-IFN-α2b (flexible linker, GGGGS (SEQ ID NO:22)), HSA-IFN-α2b (rigid linker, PAPAP (SEQ ID NO:23)), HSA-IFN-α2b (rigid linker, AEAAAKEAAAKA (SEQ ID NO:24)), PGA-Rths (a flexible linker, (GGGGS)ₙ (n=1, 2, 4), SEQ ID NO:25), interferon-γ-gp120) (rigid linker, (Ala-Pro)ₙ (10-34 aa) (SEQ ID NO:26)), GSF-S-S-Tf (cleavable linker, disulfide), IFN-α2b-HAS (cleavable, disulfide). A linker can enable targeting such as FIX-albumin (cleavable, VSQTSKLTRIAETVFPDV (SEQ ID NO:27)), LAP-IFN-β (cleavable, PLG↓LWA, SEQ ID NO: 28)), MazE-MazF (cleavable linker, RVLAEA: EDVVCCISMSY (SEQ ID NO:29) GGIEGRIGS (SEQ ID NO:30)), Immunotoxins (cleavable linkers, TRHRQPRIGWE (SEQ ID NO: 31), AGNRVRRISVG (SEQ ID NO:32), RRRRRRRIRIRd (SEQ ID NO:33), Immunotoxin (cleavable, GFLGĮ, SEQ ID NO:34)). A linker can alter a protein's PK, e.g., a dipepetide such as LE, G-CSF-Tf and hGH-Tf (a rigid linker, A(EAAAK)₄ALEA(EAAAK)₄A (SEQ ID NO:35)). See, e.g., Chen X, Zaro J L, Shen W C. Fusion protein linkers: property, design and functionality. Adv Drug Deliv Rev. 2013 October: 65 (10): 1357-69.

### Thiazole Synthetases

Thiazole synthetases, such as TbtE (a FMN-dependent oxidoreductase, NCBI accession WP_013130813.1), TbtF (an ocin-ThiF domain NCBI accession WP_206207102.1), TbtG (a YcaO type cyclodehydratase NCBI accession WP_206207103.1), and/or TbtD (NCBI accession WP_013130812.1: a lantibiotic dehydratase C-terminal domain-containing protein) can be used in the methods described herein. In an embodiment, a thiazole synthetase can comprise 80, 85, 90, 95, 96, 97, 98, 99% or more sequence identity to NCBI accession WP_013130813.1, WP_206207102.1, WP_206207103.1, or WP_013130812.1.

### Methods of Making a Pyridine-Based Macrocyclic Peptide

In an aspect, methods of making pyridine-based macrocyclic peptides are provided. A substrate comprising a leader (e.g., SEQ ID NO:1-9) and core (e.g., SEQ ID NO:10-12) as described herein or a substrate comprising a leader, core, linker, and handle region can be contacted with MroB, MroC, and MroD.

The MroB, MroC, and MroD can be used individually or can be fused to a tag, such as an affinity tag. A substrate can further comprise a linker region and a handle region at the C-terminus of the core region. In an aspect, rings with 14 to 23 members (e.g., 14, 15, 16, 17, 18, 19, 20, 21, 22, or 23 members) can be made.

In an aspect, a method of making pyridine-based macrocyclic peptides is provided. A first substrate comprising a leader, e.g.;

(SEQ ID NO: 36)
MDNVVTEAAEFADLDIDDFDLAVDEELAALSVGGLGNTEVGA,
and

a core sequence, e.g.,
SCNCFCYICCSX₁LI (SEQ ID NO:37), wherein X₁ is Y, W, F, or H or
SCX₂CX₂CX₂ICCSX₁LI (SEQ ID NO:43), wherein X₁ is Y, W, F, or H, and wherein X₂ is any amino acid,
can be contacted with the heterocycle synthetase, TbtE, TbtF, TbtG, TbtD, combinations thereof, or homologous synthetases (e.g., all proteins discussed in the “Thiazole Synthetases” section above). This results in a second substrate that is formed as follows:
a leader of MDNVVTEAAEFADLDIDDFDLAVDEELAALSVGGLGNTEVGA (SEQ ID NO:36); and
a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof, or a core sequence of SX₁X₃ X₁X₃ X₁X₃I X₁ X₁SX₂LI, (SEQ ID NO:44) wherein X₂ is Y, W, F, or H, wherein each of X₁ are thiazole, thiazoline, oxazole, oxazoline, methyloxazole, methyloxazoline, or combinations thereof, and wherein X₃ is any amino acid. The second substrate is contacted with MroB, MroC, and MroD. The first substrate, the second substrate, or both the first and second substrates can further comprise a linker region and a handle region at the C-terminus of the core region. The MroB, MroC, and MroD can be used individually, or each can be fused to a tag, such as an affinity tag.

In an aspect, a method of making pyridine-based macrocyclic peptides comprising contacting a substrate, e.g., a substrate having a leader of:
MDNVVTEAAEFADLDIDDFDLAVDEELAALSVGGLGNTEVGA (SEQ ID NO:36); and a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof, or a core sequence of SX₁X₃ X₁X₃ X₁X₃I X₁ X₁SX₂LI, (SEQ ID NO:44) wherein X₂ is Y, W, F, or H, wherein each of X₁ are thiazole, thiazoline, oxazole, oxazoline, methyloxazole, methyloxazoline, or combinations thereof, and wherein X₃ is any amino acid, with MroB, MroC, and MroD. The substrate can further comprise a linker region and a handle region at the C-terminus of the core region. The MroB, MroC, and MroD can be used individually or each can be fused to a tag, such as an affinity tag. The pyridine-based macrocyclic peptides can comprise one or more thiazole, thiazoline, oxazole, oxazoline, methyloxazole, or methyloxazoline groups.

In an aspect, a substrate can be contacted with MroB, MroC, and/or MroD (each optionally with a tag, such as an affinity tag) in the presence of ATP (e.g., about 4, 5, 6, 7, 8, or 9 mM), L-Glu (e.g., about 0.75, 1.0, or 1.25 mM), GluRS (e.g., from M. rosaria or T. bispora) and tRNAGlu (CUC) (e.g., about 1, 2, 3, 4 or 5 μM from M. rosaria or T. bispora).

The methods can be used in intermolecular cyclization and intramolecular cyclization reactions.

For intermolecular reactions two substrates can be used to generate pyridine-based macrocyclic peptides. In an example a first substrate can be:
VESLTAGHGMTEVGADhaX1 (SEQ ID NO:41), wherein X₁ is thiazole, thiazoline, oxazole, oxazoline, methyloxazole, or methyloxazoline; and a second substrate can be:
Ac-VX₁X₂DhaX₃Dha (SEQ ID NO:42), wherein X₁ and X₂ and X₃ are thiazole, thiazoline, oxazole, oxazoline, methyloxazole, methyloxazoline, or a combination thereof.

In an example, a pyridine-based macrocyclic peptide can be made by contacting a first substrate comprising:
VESLTAGHGMTEVGADhaX1 (SEQ ID NO:41), wherein X₁ is thiazole, thiazoline, oxazole, oxazoline, methyloxazole, or methyloxazoline; and a second substrate comprising:
Ac-VX₁X₂DhaX₃Dha (SEQ ID NO:42), wherein X₁ and X₂ and X₃ are thiazole, thiazoline, oxazole, oxazoline, methyloxazole, methyloxazoline, or a combination thereof with one or more polypeptides comprising 70, 80, 90, 95, 96, 97, 98, 99%, or more sequence identity (including 100 percent sequence identity) to TbtE, TbtF, TbtG, and/or TbtD.

The compounds or compositions formed by the methods disclosed herein can be used to form compositions, which can be used to treat various diseases or conditions. The compositions can be formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, PA. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol. 52:238-311.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow; the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising.” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1 Reconstitution of Enzymatic Pyritide Production

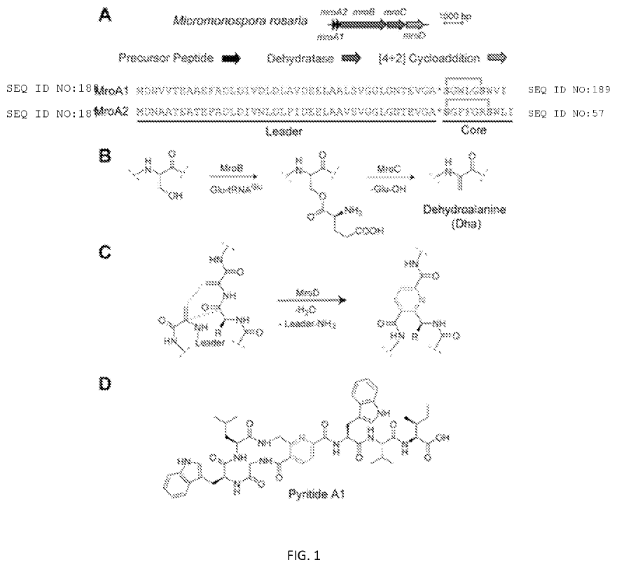

In previous work, native pyritides were accessed via total chemical synthesis or enzymatic [4+2] cycloaddition using a substrate peptide with chemically installed Dha residues.²⁸ Here, to facilitate understanding of the substrate scope of the entire pathway, we focused on the complete enzymatic biosynthesis of pyritides. We first reconstituted the activity of MroB and MroC, a split LanB-like dehydratase pair that forms two Dha residues in the MroA precursor peptides (FIG. 1). Based on membership in InterPro family IPR006827, which includes both dehydratases and enzymes with other tRNA-dependent activities, MroB (NCBI accession identifier WP_067368389.1) was expected to utilize Glu-tRNA^{Glu} to glutamylate the side chain of Ser residues. MroC (NCBI accession IPR023809, WP_083978639.1) was expected to eliminate glutamate to yield Dha. To test this hypothesis, the genes encoding MroB and MroC were cloned and expressed in Escherichia coli with maltose-binding protein (MBP) fused to the N-terminus of each protein. MBP-MroB and MBP-MroC were purified using affinity and size-exclusion chromatography; MBP-MroB was only successfully purified after co-expression with Thermobispora bispora GluRS and tRNA^{Glu} (CUC), which shares 91% sequence identity with M. rosaria tRNA^{Glu}(CUC) (FIG. 5. After purification, the precursor peptides MroA1 and MroA2 were reacted with MBP-MroB and MBP-MroC in the presence of ATP, L-Glu, T. bispora GluRS, and tRNA^{Glu} (CUC). Analysis by matrix-assisted laser desorption/ionization time-of-flight mass spectrometry (MALDI-TOF-MS) and high-resolution electrospray ionization tandem mass spectrometry (HR-ESI-MS/MS) indicated that Ser1 and Ser6/Ser7 (MroA1/MroA2) were dehydrated. Omission of MBP-MroC showed the formation of diglutamylated intermediates of MroA1 and MroA2. Didehydrated MroA1 and MroA2 were then treated with MBP-MroD (like MroC, a member of IPR023809; WP_067368384.1), yielding the expected pyritides and elimination of the leader peptide as a C-terminal carboxamide (leader-NH₂). The high-performance liquid chromatography (HPLC) and MS/MS profiles of enzymatically prepared pyritide A1 and pyritide A2 matched their corresponding standards whose structures were previously verified by ¹H NMR spectroscopy. Tolerance of the pyritide biosynthetic machinery towards single site variation. Having successfully reconstituted the enzymatic biosynthesis of pyritide A1/A2, we next examined whether residues in the core region can be substituted to generate analogs. We first varied each core position of MroA2 with amino acids of different physicochemical properties using in vitro transcription and translation, generating 52 single site variants.

These variants were subjected to the treatment of MroBCD, and the reaction outcomes were analyzed by MALDI-TOF-MS (Table 1). Table 1 summaries the MroB/C/D activity on MroA2 single core peptide variants.

TABLE 1

| | Dha | Relative MroD |
|---|---|---|
| Variation | formation | conversion |
| G2A | 2 Dha | ++ |
| G2N | 2 Dha | + |
| G2L | Mix of 1,2 | − |
| | Dha | |
| G2D | Mix of 1,2 | + |
| | Dha | |
| G2R | 2 Dha | − |
| G2W | Mix of 1,2 | − |
| | Dha | |
| F3A | 2 Dha | +++ |
| F3N | 2 Dha | +++ |
| F3L | 2 Dha | +++ |
| F3D | 2 Dha | +++ |
| F3R | 2 Dha | +++ |
| F3G | 2 Dha | +++ |
| F4A | 2 Dha | +++ |
| F4N | 2 Dha | +++ |
| F4L | 2 Dha | +++ |
| F4D | 2 Dha | +++ |
| F4R | 2 Dha | +++ |
| F4G | 2 Dha | +++ |
| G5A | 2 Dha | +++ |
| G5N | 2 Dha | +++ |
| G5L | 2 Dha | +++ |
| G5D | 2 Dha | +++ |
| G5K | 2 Dha | +++ |
| G5W | 2 Dha | +++ |
| R6A | 2 Dha | +++ |
| R6N | 2 Dha | +++ |
| R6L | 2 Dha | +++ |
| R6G | 2 Dha | +++ |
| R6W | 2 Dha | +++ |
| W8Y | 2 Dha | +++ |
| W8F | 2 Dha | +++ |
| W8L | 2 Dha | ++ |
| W8A | Mix of 1,2 Dha | + |
| W8G | Mix of 1,2 Dha | + |
| W8N | Mix of 1,2 Dha | + |
| W8D | 1 Dha | N/A (No 2 Dha |
| | | formation) |
| W8R | 1 Dha | N/A (No 2 Dha |
| | | formation) |
| L9I | 2 Dha | +++ |
| L9A | 2 Dha | ++ |
| L9G | 2 Dha | − |
| L9N | 2 Dha | − |
| L9W | 2 Dha | − |
| L9D | 2 Dha | − |
| L9R | 2 Dha | − |
| I10L | 2 Dha | +++ |
| I10V | 2 Dha | +++ |
| I10A | Mix of 0, 1, 2 | + |
| | Dha | |
| I10G | Mix of 0, 1, 2 | − |
| | Dha | |
| I10N | Mix of 0, 1, 2 | − |
| | Dha | |
| I10W | Mix of 0, 1, 2 | − |
| | Dha | |
| I10D | Mix of 0, 1, 2 | N/A (No 2 Dha |
| | Dha | formation) |
| I10R | Mix of 0, 1, 2 | − |
| | Dha | |

MroD relative activity was qualitatively estimated by comparing the intensity of leader peptide and remaining didehydrated intermediates. +++ indicates enzyme activity roughly equal to wild-type MroA2 (major species are ejected leader peptides and produced macrocycles; insignificant amount of remaining intermediates are observed); ++ indicates modestly reduced enzyme activity (both significant amount of ejected leader peptides and intermediates are observed); + indicates severely reduced enzyme activity (a high-intensity peak of remaining didehydrated intermediate, a low-intensity peak of ejected leader peptide, and observable produced macrocycles); − indicates no detectable enzyme activity (no macrocycles observed); N/A=not applicable.

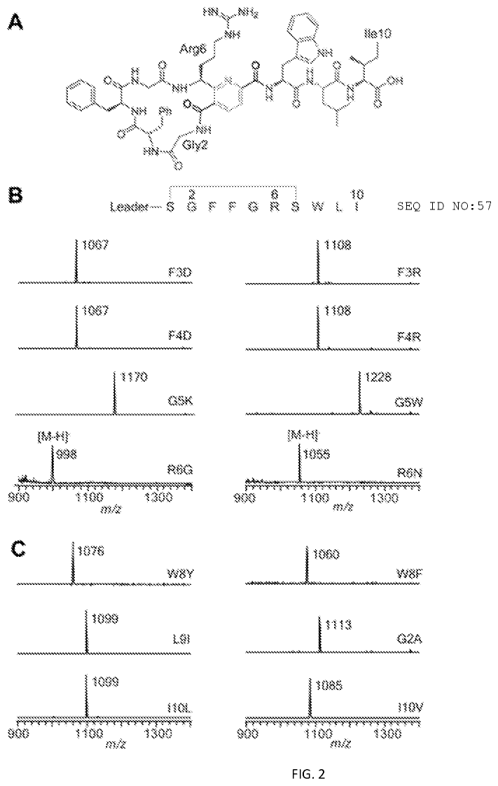

Only conservative substitutions were well tolerated at Gly2 (G2A), Trp8 (W8Y and W8F), Leu9 (L91 in MroA2) and Ile10 (I10L and I10V) (FIG. 2) for overall pyritide biosynthesis. Other Trp8 (W8G, W8A, W8D, W8N, W8R) and Ile10 (I10G, I10A, I10N, I10D, I10W) variants resulted in inefficient dehydration and macrocyclization while didehydrated peptides with non-conservative substitutions at Gly2 (G2D, G2L, G2N, G2W, G2R) and Leu9 (L9D, L9R, L9G, L9W, L9N) were poor substrates for macrocyclization. In contrast, all examined single substitutions of the ring positions (Phe3, Phe4, Gly5, Arg6) yielded the expected macrocycle.

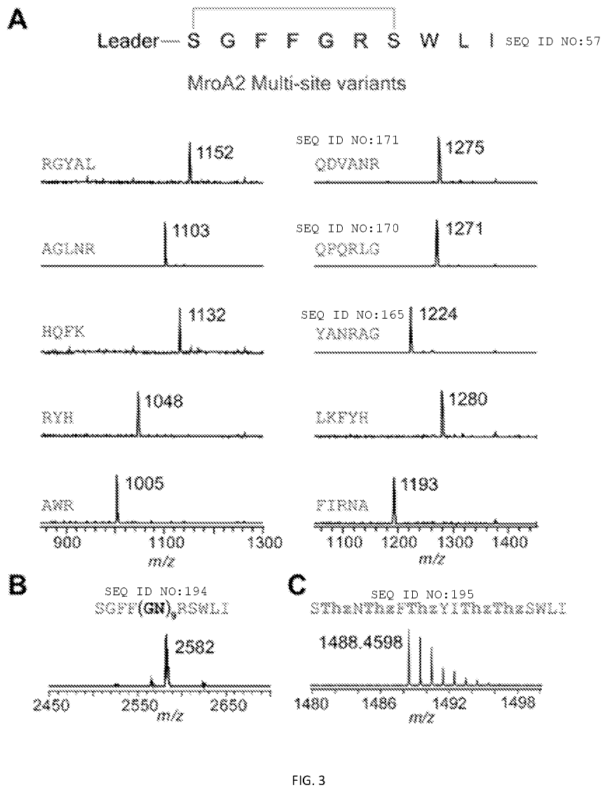

Tolerance of the biosynthetic machinery towards multi-site variation and ring expansion and contraction. Encouraged by the substrate flexibility in the ring, we next expanded the size and sequence of the macrocycle by inserting 56 different sequences varying in length from 3-6 residues between the two Ser residues involved in pyridine formation; Gly2 was retained (FIG. 3, Table 2). These substrate variants were treated with MroBCD, and the products were analyzed by MALDI-TOF-MS (and HR-ESI-MS/MS). Table 2 summarizes the MroB/C/D activity on MroA2 multi-site variants. The varied region is bolded in the precursor peptide sequence shown below:

(SEQ ID NO: 57)
f-MRRRGSMDNVVTEAAEFADLDIVDLDLAVDEELAALSVGGLGNTEV
GA (SEQ ID NO: 56)|SGFFGRSWLI

TABLE 2

| | | | Sequence of | | | | Relative |
|---|---|---|---|---|---|---|---|
| | Core peptide | SEQ | the varied | SEQ | Ring | Dha | MroD |
| Number | sequence | ID NO: | region | ID NO: | size | formation | conversion |
| MroA2 | SGFFGRSWLI | 57 | FFGR | 239 | 17 | 2 Dha | +++ |
| 1 | SGLGHSWLI | 58 | LGH | | 14 | 2 Dha | + |
| 2 | SGRANSWLI | 59 | RAN | | 14 | 2 Dha | − |
| 3 | SGVKFSWLI | 60 | VKF | | 14 | 2 Dha | + |
| 4 | SGVKADSWLI | 61 | VKAD | 240 | 17 | 2 Dha | − |
| 5 | SGPKGSWLI | 62 | PKG | | 14 | 2 Dha | +++ |
| 6 | SGAWRSWLI | 63 | AWR | | 14 | 2 Dha | +++ |
| 7 | SGHAAHSWLI | 64 | HAAH | 241 | 17 | 2 Dha | +++ |
| 8 | SGMPRGSWLI | 65 | MPRG | 242 | 17 | 2 Dha | +++ |
| 9 | SGNERGSWLI | 66 | NERG | 243 | 17 | 2 Dha | +++ |
| 10 | SGKLGFSWLI | 67 | KLGF | 244 | 17 | 2 Dha | + |
| 11 | SGGRENSWLI | 68 | GREN | 245 | 17 | 2 Dha | ++ |
| 12 | SGRYHSWLI | 69 | RYH | | 14 | 2 Dha | +++ |
| 13 | SGAQQHSWLI | 70 | AQQH | 246 | 17 | 2 Dha | +++ |
| 14 | SGVKAWSWLI | 71 | VKAW | 247 | 17 | 2 Dha | +++ |
| 15 | SGHWANSWLI | 72 | HWAN | 248 | 17 | 2 Dha | +++ |
| 16 | SGKAPGRSWLI | 73 | KAPGR | 249 | 20 | 2 Dha | +++ |
| 17 | SGAGLNRSWLI | 74 | AGLNR | 250 | 20 | 2 Dha | +++ |
| 18 | SGDGWRSWLI | 75 | DGWR | 251 | 17 | 2 Dha | +++ |
| 19 | SGKGKQASWLI | 76 | KGKQA | 252 | 20 | 2 Dha | - |
| 20 | SGIYHNSWLI | 77 | IYHN | 253 | 17 | 2 Dha | +++ |
| 21 | SGPMHYSWLI | 78 | PMHY | 254 | 17 | 2 Dha | +++ |
| 22 | SGHQFKSWLI | 79 | HQFK | 255 | 17 | 2 Dha | +++ |
| 23 | SGIKNDASWLI | 80 | IKNDA | 256 | 20 | 2 Dha | +++ |
| 24 | SGQGRNVSWLI | 81 | QGRNV | 257 | 20 | 2 Dha | + |
| 25 | SGRGYALSWLI | 82 | RGYAL | 258 | 20 | 2 Dha | ++ |
| 26 | SGMARYSWLI | 83 | MARY | 259 | 17 | 2 Dha | + |
| 27 | SGDDRDGGSWLI | 84 | DDRDGG | 163 | 23 | Mix of 1, 2 | + |
| | | | | | | Dha | |
| 28 | SGIAKAGQSWLI | 85 | IAKAGQ | 164 | 23 | 2 Dha | 1 |
| 29 | SGKALEFSWLI | 86 | KALEF | 260 | 20 | 2 Dha | − |
| 30 | SGPRANYSWLI | 87 | PRANY | 261 | 20 | 2 Dha | ++ |
| 31 | SGFIRNASWLI | 88 | FIRNA | 262 | 20 | 2 Dha | ++ |
| 32 | SGNAWIKSWLI | 89 | NAWIK | 263 | 20 | 2 Dha | +++ |
| 33 | SGYANRAGSWLI | 90 | YANRAG | 165 | 23 | 2 Dha | +++ |
| 34 | SGERGMGKSWLI | 91 | ERGMGK | 166 | 23 | 2 Dha | +++ |
| 35 | SGREFANSWLI | 92 | REFAN | 264 | 20 | 2 Dha | + |
| 36 | SGAVRFNGSWLI | 93 | AVRFNG | 167 | 23 | 2 Dha | + |
| 37 | SGRVALEASWLI | 94 | RVALEA | 168 | 23 | 2 Dha | + |
| 38 | SGGYRPHSWLI | 95 | GYRPH | 265 | 20 | 2 Dha | − |
| 39 | SGRYAGWSWLI | 96 | RYAGW | 266 | 20 | 2 Dha | − |
| 40 | SGNMHPGKSWLI | 97 | NMHPGK | 169 | 23 | 2 Dha | ++ |
| 41 | SGEMHQHSWLI | 98 | EMHQH | 267 | 20 | Mix of 1, 2 | + |
| | | | | | | Dha | |
| 42 | SGQPQRLGSWLI | 99 | QPQRLG | 170 | 23 | 2 Dha | +++ |
| 43 | SGQDVANRSWLI | 100 | QDVANR | 171 | 23 | 2 Dha | +++ |
| 44 | SGLKFYHSWLI | 101 | LKFYH | 268 | 20 | 2 Dha | +++ |
| 45 | SGGWRVAHSWLI | 102 | GWRVAH | 172 | 23 | 2 Dha | + |
| 46 | SGFDRAGHSWLI | 103 | FDRAGH | 173 | 23 | 2 Dha | + |
| 47 | SGIAKEVQSWLI | 104 | IAKEVQ | 174 | 23 | 2 Dha | − |
| 48 | SGQYKFRSWLI | 105 | QYKFR | 269 | 20 | 2 Dha | +++ |
| 49 | SGHDNQARSWLI | 106 | HDNQAR | 175 | 23 | 2 Dha | +++ |
| 50 | SGYRRRAGSWLI | 107 | YRRRAG | 176 | 23 | 2 Dha | +++ |
| 51 | SGPKRWGHSWLI | 108 | PKRWGH | 177 | 23 | 2 Dha | + |
| 52 | SGLQHGKYSWLI | 109 | LQHGKY | 178 | 23 | 2 Dha | − |
| 53 | SGDAMRWASWLI | 110 | DAMRWA | 179 | 23 | 2 Dha | |
| 54 | SGYHFHNVSWLI | 111 | YHFHNV | 180 | 23 | 2 Dha | − |
| 55 | SGHYKPQASWLI | 112 | HYKPQA | 181 | 23 | 2 Dha | − |
| 56 | SGYDRQYSWLI | 113 | YDRQY | 270 | 20 | 2 Dha | − |
| 24-V7R | SGQGRNRSWLI | 114 | QGRNR | 271 | 20 | 2 Dha | +++ |
| 29-F7R | SGKALERSWLI | 115 | KALER | 272 | 20 | Mix of 1,2 | ++ |
| | | | | | | Dha | |
| 35-N7R | SGREFARSWLI | 116 | REFAR | 273 | 20 | 2 Dha | +++ |
| 38-H7R | SGGYRPRSWLI | 117 | GYRPR | 274 | 20 | 2 Dha | +++ |
| 39-W7R | SGRYAGRSWLI | 118 | RYAGR | 275 | 20 | 2 Dha | +++ |
| 47-Q8R | SGIAKEVRSWLI | 119 | IAKEVR | 182 | 23 | 2 Dha | − |
| 52-Y8R | SGLQHGKRSWLI | 120 | LQHGKR | 183 | 23 | 2 Dha | − |
| 53-A8R | SGDAMRWRSWLI | 121 | DAMRWR | 184 | 23 | 2 Dha | − |
| 54-V8R | SGYHFHNRSWLI | 122 | YHFHNR | 185 | 23 | 2 Dha | − |
| 55-A8R | SGHYKPQRSWLI | 123 | HYKPQR | 186 | 23 | 2 Dha | − |
| 56-Y7R | SGYDRQRSWLI | 124 | YDRQR | 276 | 20 | 2 Dha | +++ |

MroD relative activity was qualitatively estimated by comparing the intensity of leader peptide and remaining didehydrated intermediates. +++ indicates enzyme activity roughly equal to wild-type MroA2 (major species are ejected leader peptides and produced macrocycles: insignificant amount of remaining intermediates are observed); ++ indicates modestly reduced enzyme activity (both significant amount of ejected leader peptides and intermediates are observed); + indicates severely reduced enzyme activity (a high-intensity peak of remaining didehydrated intermediate, a low-intensity peak of ejected leader peptide, and observable produced macrocycles); − indicates no detectable enzyme activity (no macrocycles detected).

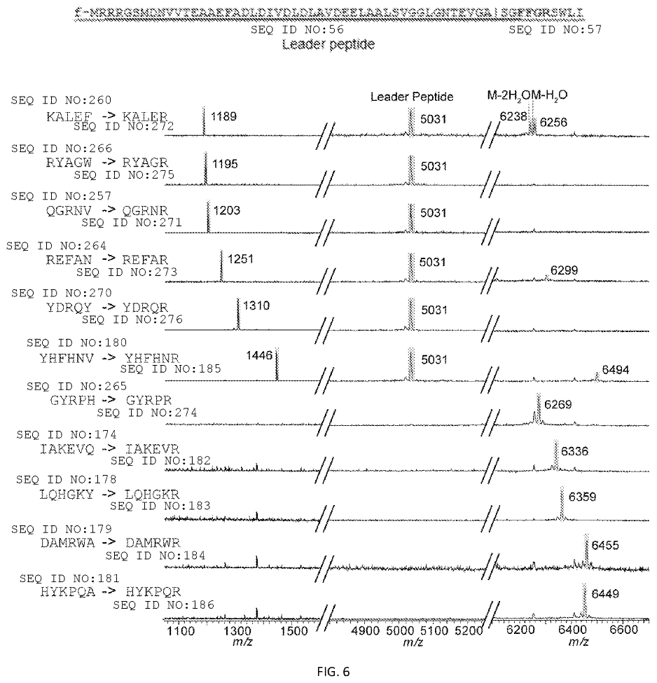

All 56 variants successfully yielded two Dha residues after treatment with MroBC, illustrating the contrast of this enzyme pair compared to dehydratases from thiopeptide BGCs that often require prior introduction of specific azoles.^{24,27,39} Reactions including MroD demonstrated that 44 out of 56 didehydrated substrates were macrocyclized (Table 2). We did not observe trends separating substrates and non-substrates of MroD in our data set, except that all variants containing Arg or Lys immediately upstream of the C-terminal Dha (equivalent to Arg6 in MroA2) were processed. Hence, positively charged residues at this position are beneficial but not essential. To examine whether an Arg residue at this position would turn non-substrates into substrates, Arg was introduced in eleven peptides that previously were poor or non-substrates for macrocyclization (FIG. 6). Six were cyclized, showing that Arg at this position contributes but is not sufficient to render any sequence a substrate.

We then examined whether Thr at this position would be preferred due to its prevalence in natural variants (FIG. 7). In all examined substrates this Thr was bypassed as a site of MroBC-catalyzed dehydration, and six out of ten didehydrated Thr-containing precursors were poor or non-substrates for macrocyclization by MroD. Thus, unlike Arg. Thr preceding the second Ser in the core peptide does not facilitate efficient pyritide formation by MroBCD but may be preferable for catalysis by other natural homologs. Further elucidation of the substrate tolerance of MroD will require structural information on core peptide binding. Nonetheless, our data show that whereas some positions are intolerant to variation, much of the precursor peptide tolerates a wide range of substitution, including multiple positively or negatively charged residues.

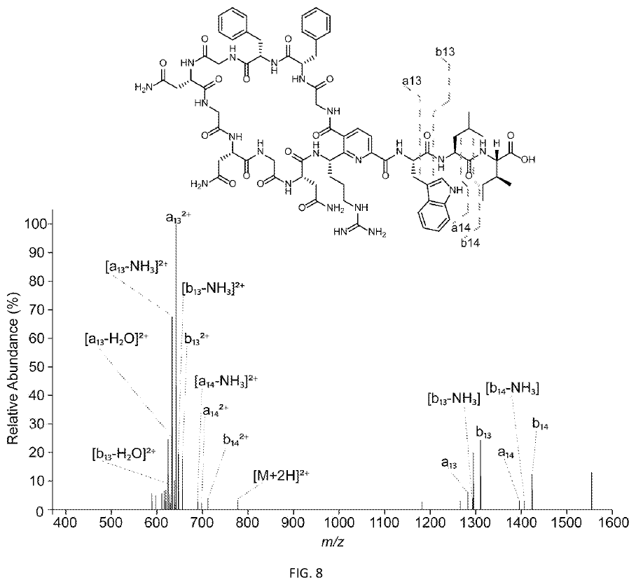

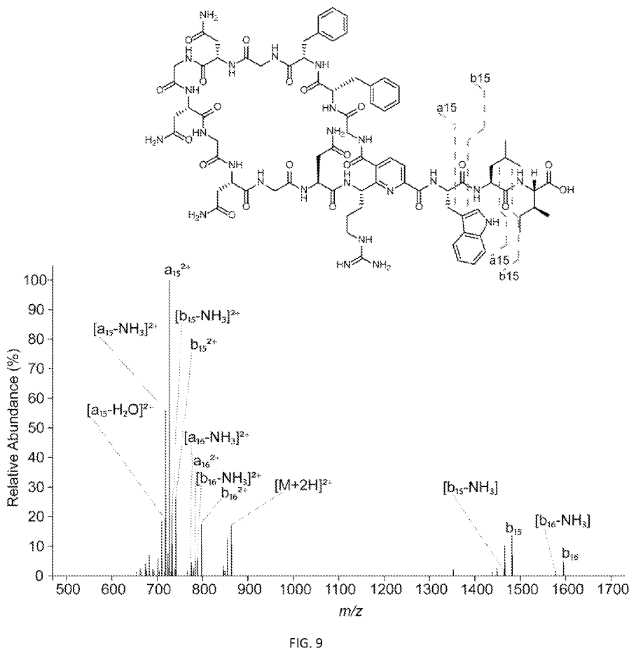

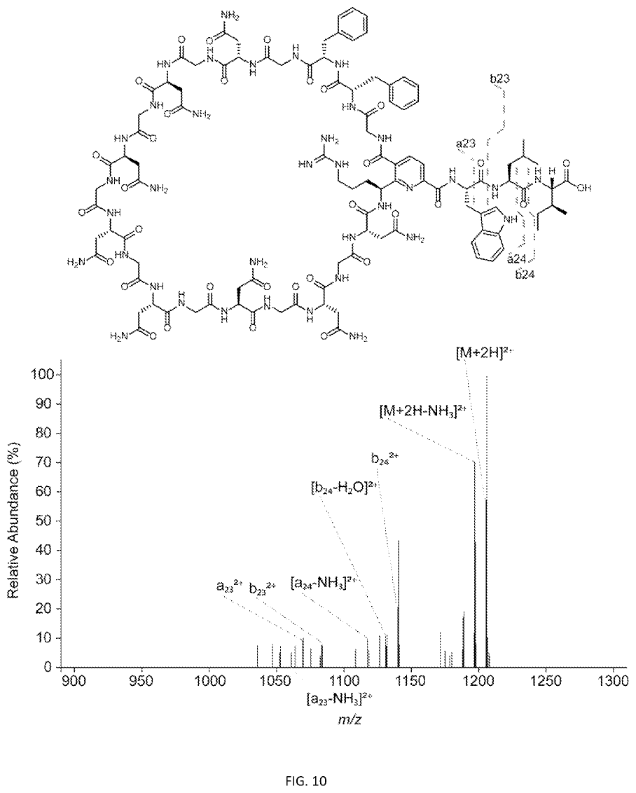

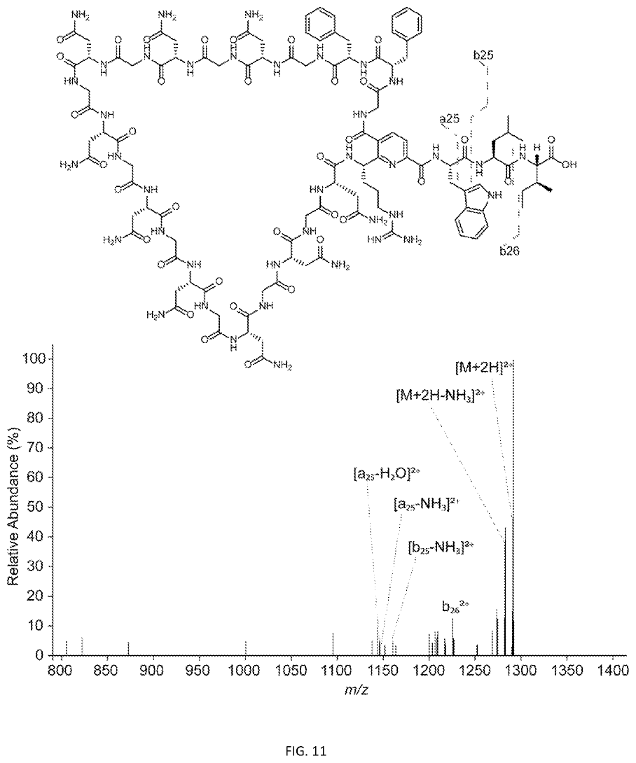

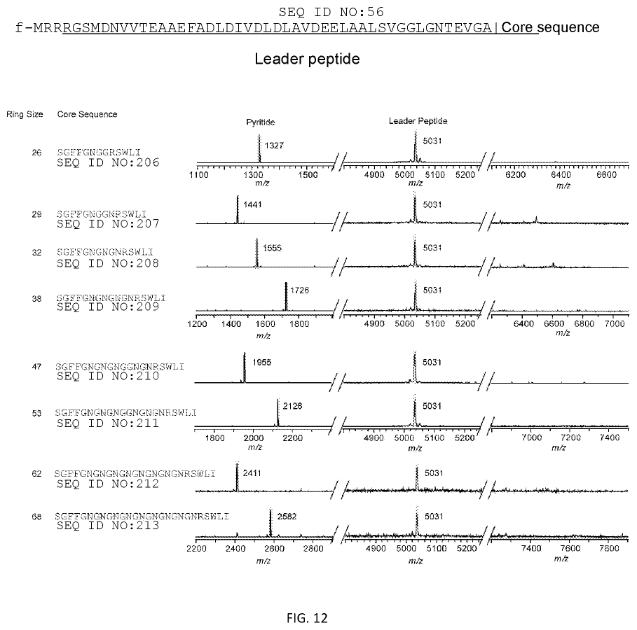

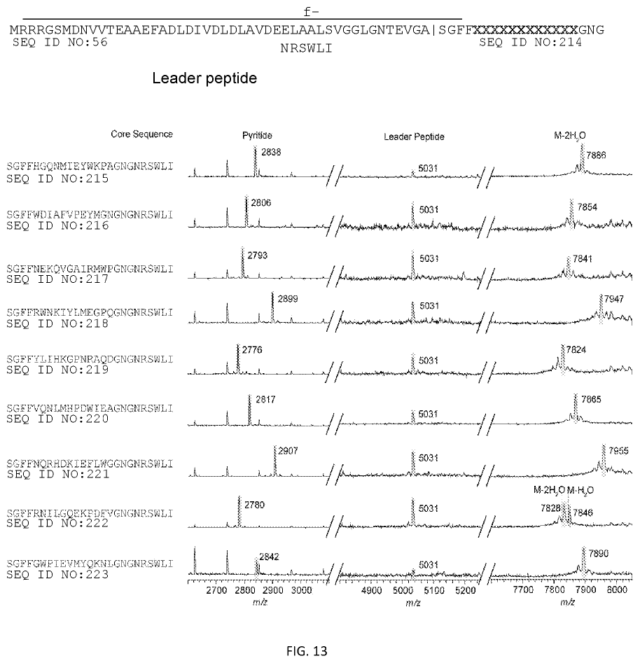

Pyritides A1 and A2 have 14- and 17-membered rings, respectively. Our substrate engineering efforts show MroBCD can form 14-23 membered rings with diverse sequences (Table 1). We examined whether ring size can be further contracted or expanded. Two (Phe4. Gly5) and three residues (Phe3. Phe4. Gly5) could be deleted without effecting the dehydration by MroBC, but MroD did not cyclize the dehydrated intermediates to form 8- and 11-membered rings. Thus, the smallest ring size achieved in our dataset is a 14-membered ring. Conversely, larger ring sizes were readily accessed including a pyritide macrocycle of 68-atoms via a 17-residue insertion of a Gly-Asn repeat, the longest attempted insertion (FIGS. 8-12). Gly-Asn repeats were initially chosen due to their established usage as hydrophilic flexible linkers⁴⁰ and were preferred in this work over popular Gly-Ser repeats⁴¹⁻⁴⁴ as they may lead to extra dehydrations and potentially complicate downstream data analysis. We subsequently examined whether MroBCD tolerates large rings with sequences different from Gly-Asn repeats through randomization. All examined sequences successfully formed 62-membered macrocycles albeit didehydrated intermediates were also detected (FIG. 13).

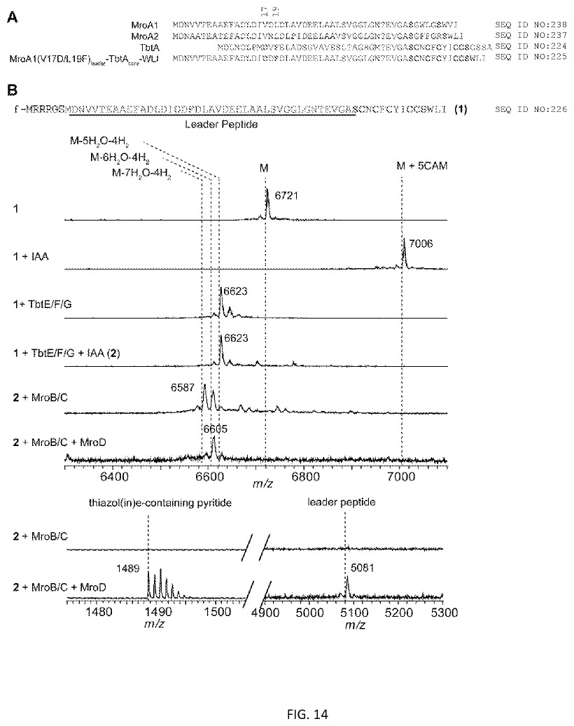

Use of MroBCD and TbtEFG for Thiopeptide formation. We next investigated whether post-translational modification can be performed on residues inside the pyritide macrocycle. We chose thiazole formation from Cys residues to assess the feasibility of using MroBCD as a platform for thiopeptide engineering. Thus, we inserted the core sequence of the thiomuracin macrocycle (with four C-terminal residues deleted) between the MroA1 leader peptide and the three C-terminal MroA residues (Trp-Leu-Ile) that were shown above to be important for MroBCD activity: The resulting core sequence shares no similarity with the wild-type sequence (FIG. 14). In addition, in the leader peptide of this non-natural substrate we incorporated residues previously identified as critical for the thiazole synthetase TbtEFG (NCBI accession identifier TbtE WP_013130813.1. TbtF WP_206207102.1. TbtG WP_206207103.1).²⁴ All Cys residues in the designed substrate peptide were successfully converted to thiazole/thiazoline residues after treatment with TbtEFG and the macrocycle was formed upon reaction with MroBCD (FIG. 3C. FIG. 14), opening possibilities to access diverse chemical space of both thiopeptides and pyritides.

Mechanism of substrate recognition. The broad substrate tolerance, including the ability to significantly expand the size of the macrocycle, combined with the observed importance of the C-terminal tripeptide for catalysis, suggested that MroBCD relies on both the leader region and the C-terminal motif for substrate binding. We tested this hypothesis through analysis of substrate binding to MroB and MroD. Substrate binding to MroC was not investigated as glutamate elimination activity was consistently observed with the substrate variants suggesting elimination activity is not limiting. This finding agrees with recent reports showing MroC homologs recognize glutamylated Ser/Thr rather than a specific peptide sequence.^{39,45} Sequence alignment of pyritide precursor peptides indicated that the first 12 residues in the leader region are not conserved and thus are unlikely to be critical for binding (FIG. 7). Indeed, a variant of MroA1 in which the first 12 residues were deleted (termed Δ12MroA1) SDLDIVDLDLAVDEELAALSVGGLGNTEVGA|SGWLGSWVI (SEQ ID NO: 55) underwent full dehydration and macrocyclization. Fluorescence polarization (FP) measurements indicated that Δ12MroA1 N-terminally labeled with fluorescein (fluorescein-Δ12MroA1) displayed high affinity toward MBP-MroB and MBP-MroD (K_{D}) MroB≈60 nM. K_{D} MroD≈12 nM). Neither the leader nor the core regions efficiently displaced the labeled precursor peptide (Table 1), confirming that MroB and MroD require both for avid binding. We also investigated a panel of MroA1 variants by competition FP assays with fluorescein-412MroA1 (Table 1). The binding data with the variants also confirm the importance of the C-terminal tripeptide for MroB (Trp7) and MroD (Trp7. Val8, and Ile9) binding. To determine if the C-terminal carboxylate is important, we evaluated the binding of MroB to the methyl ester variant of A12MroA1, which resulted in ˜8-fold loss in binding affinity (Table 1). Thus, both binding and activity data point to recognition of the leader peptide as well as the C-terminal tripeptide.

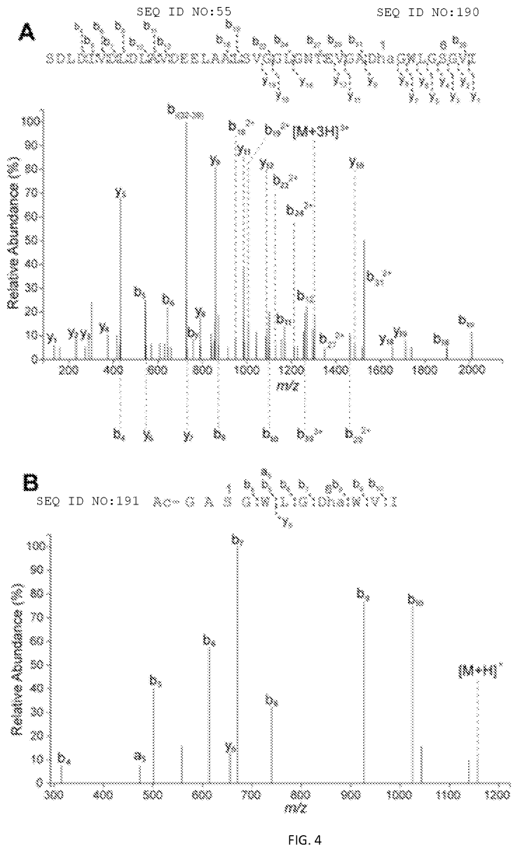

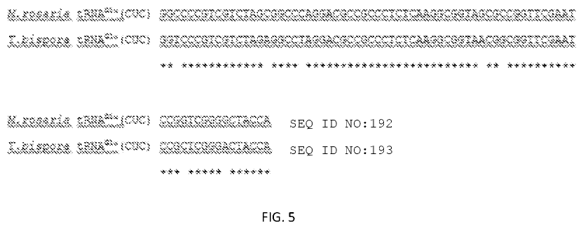

With the support for two-site recognition by MroB, we investigated how each site contributed to the overall dehydration of MroA1 and MroA2. MroBC assays followed by LC-MS/MS analysis revealed that only Ser1 is predominantly dehydrated in Δ12MroA1 W7G while only Ser6 is dehydrated in Gly Ala-MroA1core (FIG. 4). These data suggest that the leader peptide is more important for dehydration at Ser1 and the C-terminal tripeptide is more important for dehydration at Ser6. Analogously, the MroA2 variants S7G/W8G and S7G/110G were completely dehydrated at Ser1, whereas MroA2-S1G/W8G and MroA2-S1G/W10G were inefficiently dehydrated at Ser7. Dehydration of both MroA2-SIG and MroA2-S7G went to completion, indicating that the two dehydrations are independent of one another.

We fully reconstituted enzymatic pyritide biosynthesis in vitro, enabling in-depth characterization of the substrate selectivity of the dehydratase MroBC and the [4+2] cycloaddition enzyme MroD. The enzymatic macrocyclization proved compatible with in vitro translation, presenting a powerful platform for macrocyclic peptide library construction. Our data support a model in which these enzymes recognize both the leader peptide and the C-terminal tripeptide. The leader peptide is more important for dehydration at the N-terminal Ser in the core, whereas the C-terminal tripeptide is more important for dehydration at Ser6/7. By keeping the leader peptide and C-terminal residues invariant, we generated pyritide analogs with diverse ring sequences and sizes (14-68 membered). These data will facilitate future efforts in the bioengineering of macrocyclic peptides with desirable properties.

### Example 2. General Materials and Methods

Reagents used for molecular biology experiments were purchased from New England BioLabs (NEB) (Ipswich, MA). Thermo Fisher Scientific (Waltham. MA), or Gold Biotechnology Inc. (St. Louis, MO). Other chemicals were purchased from Sigma-Aldrich (St. Louis, MO). Escherichia coli DH5a and BL21 (DE3) strains were used for plasmid maintenance and protein overexpression, respectively. Plasmid inserts were sequenced at ACGT Inc. (Wheeling. IL). Matrix-assisted laser desorption/ionization time-of-flight mass spectrometry (MALDI-TOF-MS) analysis was performed using a Bruker UltrafleXtreme MALDI TOF-TOF mass spectrometer (Bruker Daltonics) at the University of Illinois School of Chemical Sciences Mass Spectrometry Laboratory. MALDI-TOF-MS samples were desalted prior to analysis by using a C18 ZipTip (EMD Millipore) prior to co-crystallization in a suitable matrix. High-resolution electrospray ionization (HR-ESI) MS/MS analyses were performed using a ThermoFisher Scientific Orbitrap Fusion ESI-MS using an Advion TriVersa Nanomate 100. Liquid chromatography coupled with ESI-MS/MS (LC-ESI-MS/MS) was performed using a 6545B LC/Q-TOF MS purchased from Agilent. Lyophilization was performed using a Labconco (Kansas City. MO) freeze dryer.

### Example 3. Molecular Biology Techniques for Generation of Plasmids Encoding Precursor Peptides and Proteins

Oligonucleotides were purchased from Integrated DNA Technologies Inc. (Coralville, LA). Sequences of primers used in this study are provided in Table 3. Table 3 shows the Oligonucleotide primers used in plasmid constructions for heterologous expression in E. coli. All sequences are provided 5′ to 3′ (left to right). F indicates a forward primer, while R indicates the reverse primer. Lowercase m indicates 2′ O-methylation of the following residue. Table 3.

| Primer Name | Oligonucleotide Sequence | SEQ ID NO: |
|---|---|---|
| mbp-F | GAGGAAGAGTTGGCGAAAGATCCACGTA | 125 |
| (sequencing) | | |
| T7-F | TAATACGACTCACTATAGGG | 126 |
| (sequencing) | | |
| T7-R | GCTAGTTATTGCTCAGCGG | 127 |
| (sequencing) | | |
| mroB | GAGCGCATTCAGGGTCTGTT | 128 |
| internal | | |
| sequencing 1 | | |
| mroB | TGCTTTGTGCAGCAGGTTCC | 129 |
| internal | | |
| sequencing 2 | | |
| mroC | GTGCTGGTATTCACTGAAGC | 130 |
| internal | | |
| sequencing | | |
| mroD | TGTTGACTCGTTCCCGCTCA | 131 |
| internal | | |
| sequencing | | |
| mroA1 | CTTCCAATCCCGTCGTCGTGGATCCATGGACAACGTCGTG | 132 |
| Gibson-F | ACTGAAGCCGCCG | |
| mroA1 | CAGTGGTGGTGGTGGTGGTGCTCGAGTCAAATCACCCAGC | 133 |
| Gibson-R | tGCCCAGCCAACCG | |
| mroA2 | AAGGATCCATGGACAATGCGGCTACGG | 134 |
| BamHI-F | | |
| mroA2 | AACTCGAGTCAGATTAACCAGGAACGACCAAAAAATC | 135 |
| XhoI-R | | |
| mroB | CAGAATACGTGGTAGAGATTGGACTGCCATGACTCGAGTC | 136 |
| Gibson F- | GACAAGCTTGCGG | |
| Backbone | | |
| mroB | GTGGGCGGTGCCGGCATGGATCCGGATTGGAAGTACAGGT | 137 |
| Gibson R- | TC | |
| Backbone | | |
| mroB | GAACCTGTACTTCCAATCCGGATCCATGCCGGCACCGCCC | 138 |
| Gibson F- | AC | |
| Insert | | |
| mroB | CCGCAAGCTTGTCGACTCGAGTCATGGCAGTCCAATCTCT | 139 |
| Gibson R- | ACCACGTATTCTG | |
| Insert | | |
| mroC | AAGGATCCATGTTACGTCACCGCGGTCAAG | 140 |
| BamHI-F | | |
| mroC XhoI-R | AACTCGAGTTATGGTGTCTCTTGCTCTGGCAC | 141 |
| mroD | AAGGATCCATGACACGTACCCCGCCG | 142 |
| BamHI-F | | |
| mroD XhoI-R | AACTCGAGTCAGCGACGGTCGTTGACAAAC | 143 |
| A12mroA1 | GGATCTGAGAACCTGTACTTCCAATCCGATTTGGACATTG | 144 |
| Gibson-F | TTGATCTGGACCT | |
| A12mroA1 | CAGTGGTGGTGGTGGTGGTGCTCGAGTCAAATCACCCAGC | 145 |
| Gibson-R | TGCCCAGCCAACCG | |
| A12mroA1 | GGATCTGAGAACCTGTACTTCCAATCCGATTTGGACATTG | 146 |
| W7G | TTGATCTGGACCT | |
| Gibson-F | | |
| A12mroA1 | CAGTGGTGGTGGTGGTGGTGCTCGAGTCAAATCACGCCGC | 147 |
| W7G | TGCCCAGCCAACC | |
| Gibson-R | | |
| 412mroA1 | GGATCTGAGAACCTGTACTTCCAATCCGATTTGGACATTG | 148 |
| V8G | TTGATCTGGACCT | |
| Gibson-F | | |
| A12mroA1 | CAGTGGTGGTGGTGGTGGTGCTCGAGTCAAATGCCCCAGC | 149 |
| V8G | TGCCCAGCC | |
| Gibson-R | | |
| 412mroA1 | GGATCTGAGAACCTGTACTTCCAATCCGATTTGGACATTG | 150 |
| 19G Gibson-F | TTGATCTGGACCT | |
| 151412mro | GCTGGGCAGCTGGGTGGGCTGACTCGAGCACCACCACCAC | 151 |
| A1 19G | CACCACTG | |
| Gibson-R | | |
| mroA2 W8G | GATTTTTTGGTCGTTGTGGGTTAATCTGACTCGAGCACCA | 152 |
| (S1C/S7C)-F | CCACCAC | |
| mroA2 W8G | GAGTCAGATTAACCCACAACGACCAAAAAATCCGCACGCA | 153 |
| (S1C/S7C)-R | CC | |
| mroA2 I10G | GTTGTTGGTTAGGCTGACTCGAGCACCACCACCACCACC | 154 |
| (S1C/S7C)-F | | |
| mroA2 I10G | GCTCGAGTCAGCCTAACCAACAACGACCAAAAAATCCGCA | 155 |
| (S1C/S7C)-R | C | |
| T. bispora | AATTCCTGCAGTAATACGACTCACTATAGGTCCCGTCGTC | 156 |
| tRNAGlu(CU | TAGAGGCCTAGGACGC | |
| C)-F | | |
| T. bispora | mUmGGTAGTCCCGAGCGGATTCGAACCGCCGTTACCGCCT | 157 |
| tRNAGlu(CU | TGAGAGGGCGGCGTCCTAGG | |
| C)-R | | |

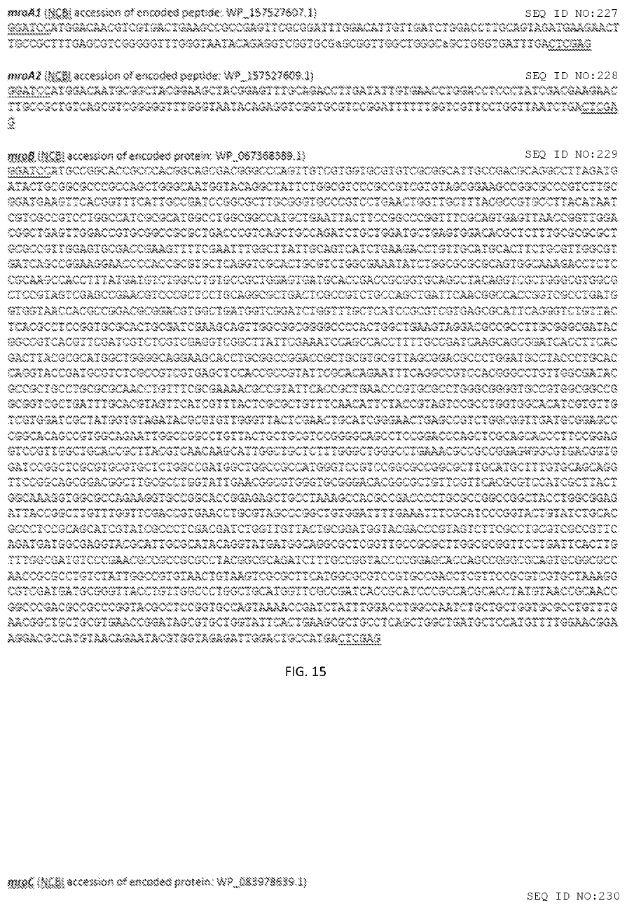

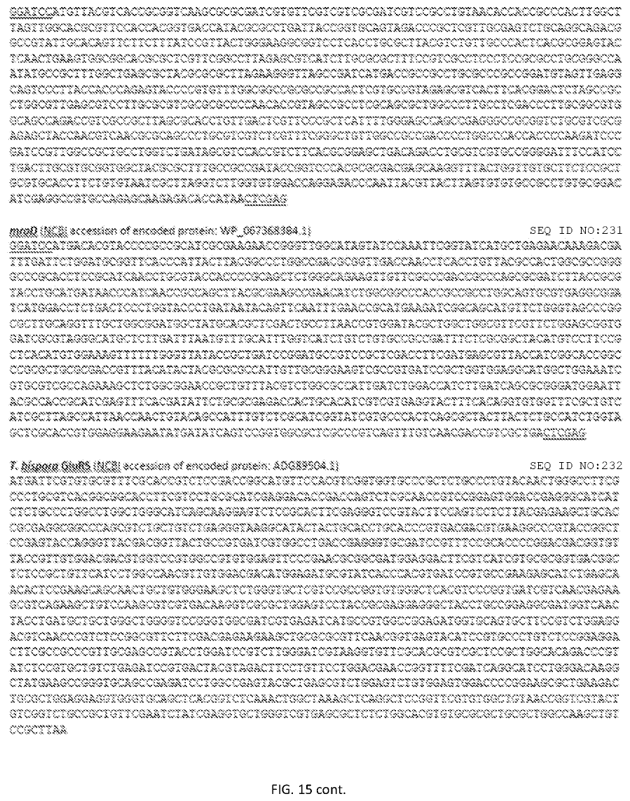

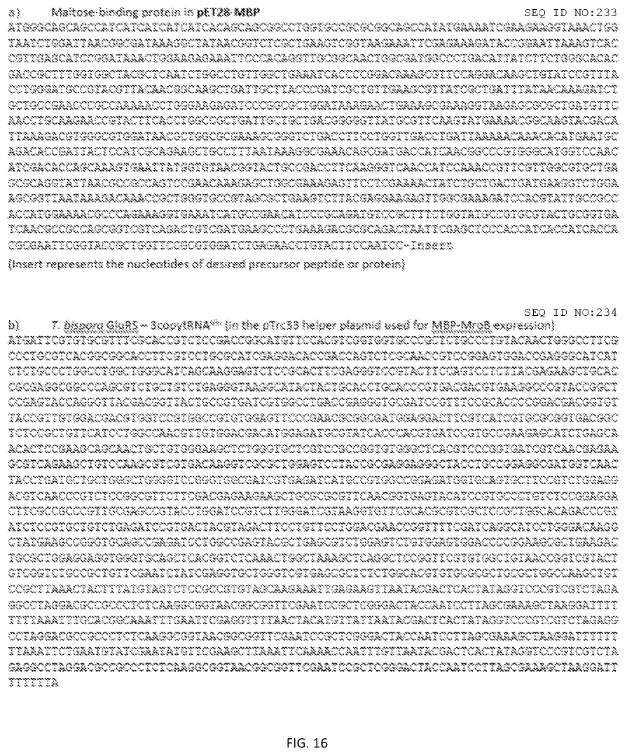

Genes optimized for recombinant expression in Escherichia coli were synthesized by Twist Bioscience in pET28 (kanamycin, Kan) vectors with BamHI and XhoI sites flanking each gene at the 5′ and 3′ ends, respectively. The GenBank locus tag and E. coli optimized sequence for each gene is provided in FIG. 15.

Our general cloning strategy involved generating DNA inserts containing DNA template encoding desired peptide-protein, respectively, and a plasmid backbone for growth in E. coli. The DNA inserts were generated by PCR (50 μL scale, 34 cycles of 95° C. denaturation 30 s, annealing 30 s, and 72° C. extension 30 s) using a Q5 polymerase kit purchased from NEB, with annealing temperature calculated from NEB Tₘ calculator. Most expression vectors derive from a modified pET28 backbone that fuses maltose-binding protein (MBP) to the N-terminus of the protein of interest. The 5′ BamHI and 3′ XhoI restriction sites were used for plasmid linearization with the exception of mroB, where Q5 PCR described above (extension time 5 min) with primer F-Backbone and R-Backbone was used. The amplified DNA inserts and digested plasmid vectors were purified using agarose gel electrophoresis [0.7% (w v)] followed by gel extraction (GeneJET).

The vectors and inserts were ligated using Gibson ligation¹ or T4 DNA ligase. If the primer used to generate inserts create BamHI and XhoI sites (Table3), ligation was done using T4 DNA ligase (NEB). Otherwise, ligation was achieved using Gibson Assembly Master Mix (NEB) at 50° C. for 1 h. Ligation reactions were used to transform chemically competent DH5a cells, which were then plated on Luria-Bertani (LB) agar plates containing 50 μg/mL kanamycin and grown at 37° C. Colonies were picked at random and grown in LB broth for 12-15 h before plasmid isolation using GeneJET Plasmid Miniprep Kit. For all MBP-Tagged precursor peptides and proteins used in this study (except for TbtE), the vector encoded a tobacco etch virus (TEV) protease-cleavable site (ENLYFQS: SEQ ID NO:281) at the N-terminus of the peptides and proteins. All recombinant constructs featuring an MBP-tag were sequenced using a custom MBP forward primer and reverse-sequenced using a T7 reverse primer as well as an internal primer when necessary (Table 3).

mroA2-W8G and mroA2-110G were generated by site-directed mutagenesis using the QuikChange method (Agilent) on the plasmid pET28-MBP-MroA2 (SIC/S7C) developed previously.

### Example 4. Generation of mroA Variants Templates DNA for In Vitro Translation

Linear double-stranded DNA encoding a T7 promoter and ribosome binding site upstream of the mroA open reading frame and mutants were synthesized by one- or multiple-step PCR from single-stranded DNA oligonucleotides using Taq polymerase (NEB). The PCR contains 10 mM Tris-HCl pH 8.3, 50 mM KCl, 1.5 mM MgCl₂, 200 μM each dNTPs, and 1 μM of the appropriate forward and reverse primers.

Generally, the protocol involved three different PCR steps: 1) Primer extension: 2) 5-cycle PCR for lengthening DNA template (multiple PCRs were performed for long DNA template) Final PCR to amplify the final PCR product that will be used for in vitro translation.

The first step involved primer extension to create an extension product with T7 promoter and RBS upstream of mroA1 leader (ExtPrimerF1). Specifically, the primer ExtPrimerF1 was mixed with Leader.R1 in the PCR mixture (100 μL scale), denatured at 95° C. (1 min) in 1 cycle, followed by 5 cycles of 54° C. annealing (1 min) and 72° C. extension (1 min).

The 5-cycle PCR was done as follows: The extension product was diluted 200-fold by the polymerase mixture and amplified using the respective forward and reverse primers (1 μM final concentration each) in a 50 μL reaction. After primer addition, the mixture was subjected to 5 cycles of 95° C. denaturation (40 s), 61° C. annealing (40 s, and 72° C. extension (40 s). Multiple 5-cycle PCRs were needed for long DNA templates.

The final PCR was done as follows: The resulting PCR product from 5-cycle PCRs was diluted 200-fold by the polymerase mixture followed by the addition of the appropriate forward and reverse primers (1 μM in final concentration each) in a 100 μL scale reaction. The new PCR mixture was then subjected to a final PCR reaction with 30 cycles of 95° C. denaturation (40 s), 61° C. annealing (40) s), and 72° C. extension (40 s). The final PCR reaction was carried out directly after the extension reaction for DNA templates that required only one-step PCR. For sequences Leader-SGRGKIQASWLI (SEQ ID NO:39) to Leader-SGANGVKTAWLI (SEQ ID NO:40) (Row 143 to Row 151, sheet MroA2variant-PCR, the final PCRs were done with the following cycle instead: 5 cycles of 95° C. denaturation (40 s), 54° C. annealing (40 s), and 72° C. extension (40 s), followed by 30 cycles of 95° C. denaturation (40 s), 61° C. annealing (40) s), and 72° C. extension (40 s).

The amplified DNA template was purified by ethanol precipitation. Specifically, in a 100 μL PCR, 10 μL of 3 M NaCl and 220 μL of EtOH was added, left on ice for 1 h, and subjected to centrifugation at 13,000×g for 20 min at 4° C. The supernatant was removed, and 500 μL of 70% EtOH was added to the resulting pellet, followed by centrifugation at 13,000×g for 10 min at 4° C. The supernatant was removed entirely, and the resulting pellet was dried by opening the cap of the Eppendorf tube (loosely covered by a Kimwipe) for 10 min. H₂O (10 μL) was then used to dissolve the DNA pellet, and this DNA solution was used for in vitro transcription/translation reactions.

### Example 5. MBP-Tagged Peptide Overexpression and Purification

E. coli BL21 (DE3) cells were transformed with a pET28 plasmid encoding the MBP-tagged peptide of interest. Cells were grown for 14-16h on LB agar plates containing 50 μg/mL kanamycin at 37° C. Single colonies were used to inoculate 10 mL of Terrific Broth (24 g/L yeast extract, 12 g/L tryptone, 0.4% glycerol (v/v), 17 mM KH₂PO₄, and 72 mM K₂HPO₄) containing 50 μg/mL kanamycin and grown at 30° C. for 14-18 h. This culture was used to inoculate 1 L of Terrific Broth (TB) containing 50 μg/mL kanamycin and grown to an optical density at 600 nm (OD₆₀₀) of 1.5-1.7. Protein expression was induced by addition of 0.4 mM isopropyl β-D-1-thiogalactopyranoside (IPTG, final) for 16 h at 16° C. At the time of induction, the culture was also supplemented with 2 mM MgCl₂ and 100 μg/mL FeSO₄·7H₂O as final concentrations. Cells were harvested by centrifugation at 4,500×g for 15 min, washed with phosphate-buffered saline (PBS: 137 mM NaCl, 2.7 mM KCl, 10 mM Na₂HPO₄, and 1.8 mM KH₂PO₄), and subjected to a second round of centrifugation. The cell pellet was flash-frozen and stored at −80° C. for a maximum of two weeks before use.

Cell pellets were resuspended in lysis buffer (50 mM HEPES-NaOH pH 7.5, 500 mM NaCl, 5% glycerol (v v), and 0.1% Triton X-100) containing 4 mg/ml lysozyme, 2 μM leupeptin, 2 μM benzamidine, and 2 μM E64 in 50 mL tubes. The tubes were placed in an ice-water bath, homogenized by sonication (30 s on, 10 s off, continued with another 30 s on, followed by 10 min periods of gentle rocking at 4° C.). Sonication was repeated another two rounds for a total of three. Insolublecellular debris was removed by centrifugation at 30,000×g for 30 min. The supernatant was then applied to a pre-equilibrated Ni-NTA resin (His-Pur, Thermo Scientific, 2 mL of resin per L of cells). The column was washed with 10 column volumes (CV) of lysis buffer followed by 15 CV of wash buffer (50 mM HEPES-NaOH pH 7.5, 1M NaCl. 30 mM imidazole. 5% glycerol). The MBP-tagged peptides were eluted using 6 CV of elution buffer (50) mM HEPES-NaOH pH 7.5, 300 mM NaCl. 250 mM imidazole. 5% glycerol. The eluent was concentrated using a 30 kDa molecular weight cut-off (MWCO) Amicon Ultra centrifugal filter (EMD Millipore) and buffer-exchanged into protein storage buffer [(50 mM HEPES pH 7.5, 300 mM NaCl. 2.5% glycerol (v/v)] using a PD-10 (Cytiva Life Sciences). Protein concentrations were estimated using 280 nm absorbance (theoretical extinction coefficients were calculated using the ExPASy ProtParam tool; web.expasy.org/protparam/protpar-ref). For cysteine-containing precursors, lysis, wash, elution, and storage buffers were supplemented with 0.5 mM tris-(2-carboxyethyl)-phosphine (TCEP).

### Example 6. Purification of Precursor Peptides after Affinity Chromatography

MBP-tagged precursor peptides (in 50 mM HEPES. 300 mM NaCl, 2.5% glycerol) were treated with TEV protease (L56V/S135G/S219V) 5 (50 mM HEPES, 300 mM NaCl, 2.5% glycerol, and 0.5 mM TCEP) with a 10:1 substrate to protease ratio at room temperature for 1 h. The mixture was then loaded to a C18 solid-phase extraction column (HyperSep C18 cartridges. Thermo Scientific) that was preequilibrated using 5 CV of acetonitrile and 5 CV of 20 mM NH₄OAc. The column was washed with 5 CV of 20 mM NH₄OAc before eluting with 80% acetonitrile. 4 mM NH₄OAc. For the 2000 mg and 5000 mg columns. 15 and 25 mL of elution were used, respectively. The collected eluant was then lyophilized and dissolved in 10-15 mL 150 mM NH₄HCO₃, subjected to centrifugation at 18.000×g for 20 min at room temperature to remove any insoluble debris before injecting on an HPLC equipped with a preparative C18 column. (VP HPLC column (preparative). NUCLEODUR C18 HTec, 5 μm, 250×10 mm). Solvent A was 20 mM NH₄OAc while solvent B was acetonitrile. The gradient was as follows: 2-30% B in 5 min. 30-70% B in 20 min. 70-2% B in 1 min. 2% B in 5 min before ending the run. The desired fractions were collected, lyophilized, resuspended in H₂O, vortex, and lyophilized again to remove any residual NH₄OAc. Before further use in FP or in vitro assays, the lyophilized powder was dissolved in 0.5× storage buffer (25 mM HEPES, 150 mM NaCl, 1.25% glycerol, and 0.25 mM TCEP, pH 7.5). The concentration of each peptide was assayed using 280 nm absorbance (theoretical extinction coefficients were calculated using the ExPASy ProtParam tool; web.expasy.org/protparam/protpar-ref) or Pierce Quantitative Colorimetric Peptide Assay (Thermo Scientific).

### Example 7. MBP-Tagged MroB Overexpression and Purification

E. coli BL21 (DE3) cells were transformed with pET28-MBP-tagged MroB and a pTrc33 plasmid encoding GluRS and three copies of tRNA^{Glu} (CUC) from Thermobispora bispora bearing a chloramphenicol marker. The GluRS and each copy of tRNA^{Glu} gene were preceded by a T7 promoter. Cells were for grown for 16-18 h on LB agar plates containing 50 μg/mL kanamycin and 25 μg/mL chloramphenicol at 37° C. Single colonies were used to inoculate 10 mL of LB or TB containing 50 μg/mL kanamycin and 25 μg/mL chloramphenicol and grown at 30° C. for 14-18 h. This culture was used to inoculate 1 L of LB or TB containing 50 μg/mL kanamycin and 25 μg/mL chloramphenicol grown to an optical density at 600 nm (OD₆₀₀) of 0.6-0.8 for LB and 1.5-1.7 for TB. Protein expression was induced by adding 0.5 mM IPTG and supplemented with 2 mM MgCl₂ as the final concentrations and proceeded for 18 h at 18° C.

Cell pellets were resuspended in lysis buffer containing 4 mg/mL lysozyme. 2 μM leupeptin. 2 μM benzamidine, and 2 μM E64 on 50 ml falcon tubes. Cells on the falcon tubes were then put on an ice-water bath, homogenized by sonication (25 s on, 10 s off, continued with another 25 s on, followed by 10 min nutation periods at 4° C.). The sonication was repeated another two times, resulting in a total of three times sonication. For cultures larger than 3 L, the cells were lysed using a high-pressure homogenizer (Avestin. Inc.). Insoluble debris was removed by centrifugation at 30,000×g for 30 min. The supernatant was then applied to a pre-equilibrated Ni-NTA resin (His-Pur. Thermo Scientific. 1 mL of resin per L of cell cultures). The column was washed with 10 column volumes (CV) of lysis buffer containing 0.5 mM TCEP, followed by 16 CV of wash buffer 1 (50 mM HEPES-NaOH pH 7.5, 1 M NaCl. 30 mM imidazole. 5% glycerol. 0.5 mM TCEP). The Ni-NTA column was then further washed by 5 CV of wash buffer 2 (50 mM HEPES-NaOH pH 7.5, 300 mM NaCl. 50 mM imidazole, 5% glycerol. 0.5 mM TCEP). MBP-MroB was eluted from the column twice, first time using 5 CV of pre-elution buffer (50) mM HEPES-NaOH pH 7.5, 300 mM NaCl. 125 mM imidazole. 5% glycerol. 0.5 mM TCEP) and 5 CV of elution buffer (50 mM HEPES-NaOH pH 7.5, 300 mM NaCl, 250 mM imidazole, 5% glycerol. 0.5 mM TCEP). As the fraction from the elution buffer contained less impurity visualized through SDS-PAGE gel, this fraction was concentrated further using a 30 kDa MWCO Amicon Ultra centrifugal filter (EMD Millipore). A buffer exchange with 1000× volume of protein storage buffer (50 mM HEPES pH 7.5, 300 mM NaCl. 2.5% glycerol (v/v). 0.5 mM TCEP) was performed. The buffer-exchanged protein batch was further purified with size exclusion chromatography by injecting it to an AKTA FPLC system equipped with a HiLoad 16/60 Superdex 200 pg column purchased from Cytiva Life Sciences. The column was preequilibrated and ran in the protein storage buffer. All fractions containing the proteins were identified using SDS-PAGE gel, collected, and concentrated using 30 kDa MWCO Amicon Ultra centrifugal filter (EMD Millipore) to 20 mg/mL. Protein concentrations were assayed using 280 nm absorbance (theoretical extinction coefficients were calculated using the ExPASy ProtParam tool; web.expasy.org/protparam/protpar-ref).

### Example 8. MBP-Tagged MroC Overexpression and Purification

E. coli BL21 (DE3) cells were transformed with pET28-MBP-tagged MroC and a chloramphenicol-resistant pACYC-Duet plasmid containing Cpn10 and Cpn60, which are chaperones from Oleispira antarctica.³ Cells were for 16-18 h on LB agar plates containing 50 μg/mL kanamycin and 25 μg/mL chloramphenicol at 37° C. Single colonies were used to inoculate 10 mL TB containing 50) μg/mL kanamycin and 25 μg/mL chloramphenicol grown at 30° C. for 14-18 h. This culture was used to inoculate 1 L of TB containing 50 μg/mL kanamycin and 25 μg/mL chloramphenicol grown to an optical density at 600 nm (OD₆₀₀) of 0.6-0.8 for LB and 1.5-1.7 for TB. The expression was then induced by adding 0.5 mM IPTG and supplemented with 2 mM MgCl₂ as the final concentrations, and proceeded for 18 h at 18° C.

The purification of MBP-MroC was done similarly to MBP-MroB with a few modifications. After the first wash with 10 CV of lysis buffer, the second wash was only done with 10 CV instead of 16 CV of wash buffer 1. 10 CV of chaperone-wash buffer (50 mM HEPES-NaOH pH 7.5, 10 mM MgCl₂, 7.5 mM ATP, and 150 mM KCl) was then applied to the column. The column was then allowed to nutate at 4° C. on a nutator for 2 h to break the interaction between the chaperone and MBP-MroC.⁴ 10 CV of wash buffer was then applied, followed by 5 CV of pre-elution buffer and 6 CV of elution buffer. Three rounds of buffer exchange from elution buffer to protein storage buffer (50 mM HEPES pH 7.5, 300 mM NaCl. 2.5% glycerol (v/v), 0.5 mM TCEP) using 10× volume of protein storage buffer in each round was performed before size exclusion chromatography by Amicon 30 kDa 15 mL. The buffer-exchanged protein batch was further purified with size exclusion chromatography in a similar manner as MBP-MroB.

### Example 9. MBP-Tagged MroD Overexpression and Purification

E. coli BL21 (DE3) cells were transformed with pET28-MBP-tagged MroD and a chloramphenicol-resistant pACYC-Duet plasmid containing Cpn10) and Cpn60, which are chaperones from Oleispira antarctica.³ The expression, affinity chromatography, and SEC were performed similar to MBP-MroC, but without applying chaperone-wash or pre-elution buffer. The wash steps included 10 CV of lysis buffer. 15 CV of wash buffer, and elution was done using 6 CV of elution buffer.

### Example 10. Expression and Purification of Thermobispora bispora GluRS

E. coli BL21 (DE3) cells were transformed with pRSF-His6-T. bispora GluRS plasmid bearing a kanamycin-resistant marker. Expression and affinity chromatography was done similarly to MBP-tagged precursor peptide. Every buffer in this purification contained 0.5 mM TCEP.

### Example 11. Expression and Purification of TEV Protease

E. coli BL21 (DE3) cells were transformed with pK793-TEV (L56V/S135G/S219V) plasmid bearing an ampicillin-resistant marker.⁵ Expression and affinity chromatography were done similarly to MBP-tagged precursor peptide, with 100 μg/mL of ampicillin or carbenicillin used instead of kanamycin. Every buffer in this purification contained 0.5 mM TCEP. The overnight preculture was subjected to centrifugation of 4000×g for 15 min, and supernatant (media) was removed, followed by resuspension in the same amount of fresh TB media prior to expression.

### Example 12. Expression and Purification of MBP-LahS_{B}

E. coli BL21 (DE3) cells were transformed with pET28a-MBP-LahS_{B} plasmids bearing a kanamycin-resistant marker.⁶ Expression and affinity chromatography were done similarly to MBP-tagged precursor peptide. Every buffer in this purification contained 0.5 mM TCEP.

### Example 13. Expression and Purification of MBP-TbtE, MBP-TbtF, and MBP-TbtG

The expression and purification of these proteins are performed according to a previously reported protocol.⁷

### Example 14. In Vitro Transcription of T. Bispora tRNA^{Glu} (CUC)

The protocol was done following a previous publication first describing the usage of T. bispora tRNA^{Glu} (CUC) in the thiopeptide thiomuracin biosynthesis.⁷ Briefly, the tRNA^{Glu} dsDNA template was generated from two overlapping synthetic deoxyoligonucleotides with sequences provided in Table 3. To make dsDNA template for in vitro transcription. 5′ overhangs were assembled using this reaction condition: NEB Buffer 2 (1×), primers (4 μM each), dNTP (100 μM each). DNA polymerase I large (Klenow) fragment (1 U/μg DNA) in a final volume of 50 μL. The reaction was incubated at 25° C. for 15 min. quenched with EDTA (10 mM) at 75° C. for 25 min, and dsDNA tRNA^{Glu} template was precipitated with cold EtOH overnight. The DNA template was then washed twice with 75% cold EtOH, and the supernatant was removed through centrifugation for 20 min at 13000×g. The pellet was then air-dried for 15 min before being dissolved in H₂O. For a 50 μL PCR scale. 10 μL H₂O was used to dissolve the DNA pellet. In vitro transcription was performed using this reaction condition: 100 mM HEPES-KOH PH 7.5, 36 mM MgCl₂·50 mM DTT, 7.5 mM each rNTPs. 2 mM spermidine-HCl. 0.1 mg/mL bovine serum albumin (RNase-Free). 0.8 U/μL Ribolock RNAse Inhibitor. 0.5 mU/μL E. coli inorganic phosphatase. (Thermo Scientific). 100 ng/μL DNA template, and 10 U/μL T7 RNA polymerase. The reaction was incubated overnight in an air chamber at 37° C.

The transcribed tRNA^{Glu} was then purified by acidic phenol extraction. Specifically. 0.05 U/μL of RNase-free DNase was added to the transcription mixture and incubated for 30 min at 37° C. The reaction mixture was then buffer exchanged with 1000× volume of 100 mM HEPES pH 7.5 by Amicon filter (30 kDa) to remove residual rNTPs. The RNA was then extracted with an equal volume of acidic phenol. The phenol phase was then back extracted with an equal volume of 300 mM NaOAc pH 5.2 and combined with the aqueous phase. This extraction was repeated one more time before the aqueous phase was extracted twice with a mixture of chloroform and isoamyl alcohol (24:1). The aqueous phase (top phase) was then collected and precipitated with 2.5 times volumes of EtOH. The supernatant was removed after 13,000×g centrifugation 15 min, and the pellet was then washed twice with 75% EtOH. The supernatant was again removed after 13,000×g centrifugation for 10 min, and air-dried for 15 min. The pellet was then redissolved in 2 mM NaOAc pH 5.2, and the concentration was assayed using 260 nm absorbance. This tRNA^{Glu} (CUC) was then used in the MroBC-catalyzed dehydration assay.

### Example 15. In Vitro Translation and Enzymatic Assays (Dehydration and Cyclization)

In a 0.65 mL protein low-binding Eppendorf tube placed on ice. 0.75 μL of purified mroA variant template DNA was mixed with 0.75 μL of Solution B and 1 μL of Solution A of PURExpress In vitro Protein Synthesis Kit (E6800L) purchased from NEB (total volume of reaction is 2.5 μL). The translation reactions were performed at 37° C. for 1 h on an aluminum block.

For a full MroBCD substrate scope investigation, a total translation volume for each variant of 7.5 μL was performed. 1.5 μL of 90 mM iodoacetamide (IAA) was added to quench dithiothreitol (DTT) in the translation reaction mixture, a thiol-based nucleophile that can react with electrophilic dehydroalanines generated from MroBC-catalyzed dehydration assays. The translation product was split into two parts with these corresponding volumes: 3 μL and 6 μL. In the 6 μL part. 10.2 μL of the enzyme mix containing enzymes and cofactors was added along with 1.8 μL 90 mM ATP (pH 7.5), which was used to initiate the reaction. The enzyme mix was made by adding these components in the following order: HEPES pH 7.5, MgCl₂, glutamate, T. bispora tRNA^{Glu} (CUC), thermostable inorganic pyrophosphatase (TIPP), TEV (L56V/S135G/S219V) (w/w ratio with a total amount of MBP-tagged protein=1:10), MBP-MroB, MBP-MroC, and T. bispora GluRS. The enzyme mix was incubated 25 min at room temperature before adding to the translation product to facilitate in situ TEV-catalyzed cleavage of MBP from MBP-MroB and MBP-MroC. The enzymatic reaction proceeded for 1 h at room temperature. Overall, the concentration of components in the 18 μL reaction mix is as follows: 50 mM HEPES pH 7.5, 5 mM MgCl₂. L-glutamate 1 mM, 3 μM T. bispora tRNA^{Glu}, 1 μM T. bispora GluRS, 2 μM MBP-MroB, 2 μM MBP-MroC, 6 mM ATP, 0.027 U/μL TIPP, and 5 mM IAA. In addition, the 3 μL part was incubated with the same buffer lacking enzymes and tRNA as a translation control.

After incubation, the enzyme reaction (18 μL) was split into two equal parts. One part was treated with MBP-MroD such that the final concentration of MBP-MroD is 3 μM, and the other part was added the same volume but with buffer lacking MBP-MroD (50 mM HEPES, 300 mM NaCl, 2.5% glycerol, 0.5 mM TCEP). The reaction further proceeded for 1.5 h at room temperature. All reaction mixture was then desalted with solid-phase extraction using Ziptip with 0.6 μL C18 resin (EMD Millipore) and crystallized on an MTP384 steel target plate using either saturated sinapic acid or 50 mg/mL Super DHB [dissolved in 60% acetonitrile and 0.1% trifluoroacetic acid (TFA)] as the matrix. The crystallized spots were then analyzed using MALDI-TOF-MS (Bruker Ultraflex). The external standard for MALDI-TOF-MS analysis is ProteoMass Peptide and Protein MALDI-MS Calibration Kit (Sigma).

In this work. TEV was used in every variant tested. However, the dehydration and cyclization of wild-type substrate (MroA2) were also observed without TEV in the assay.

### Example 16. Enzymatic Assays (Dehydration and Cyclization) with Purified Substrates

The reactions were performed similarly as described above, with 10 μM substrate concentration and without IAA. The total volume of each reaction is 100 μL.

### Example 17. Computational Generation of Random Sequences

ExPASy RandSeq tool (web.expasy.org/randseq/) was utilized to generate random peptide sequences for assessing the substrate scope of large ring formation. The composition of amino acids in the peptide sequences was specified to be 5.88% for each 17 canonical amino acids (Ala, Arg, Asp, Asn, Gln, Glu, Gly, His, Ile, Leu, Lys, Met, Phe Pro, Trp, Tyr, Val) and 0% for Cys, Ser, and Thr.

### Example 18. C-Terminal O-Methylation Using LahMet

100 μM MBP-tagged 412MroA1 was incubated with 20 μM MBP-tagged LahS_{B} in the presence of 1 mM S-adenosyl methionine (SAM) and 50 mM HEPES pH 7.5 at room temperature for 16 h.⁶ The peptides generated from a 25 ml reaction were then subjected to TEV cleavage followed by solid-phase extraction and HPLC purification, as mentioned above. Only the fractions containing the methylated peptide were collected after purification.

### Example 19. Dehydrothiolation of Cysteines in Precursor Peptides to Generate Dehydroalanines

The MBP-tagged MroA2 precursor peptide and variants (0.1 mM) after overexpression and affinity chromatography were each incubated with TEV protease (0.01 mM) for 15 min at room temperature, followed by the addition TCEP (0.2 mM), potassium carbonate (0.1 mM), and N. N-dimethylformamide (DMF) (one half of the final reaction volume). Upon addition of DMF, cleaved MBP precipitated from solution. The mixture was incubated at 37° C. with agitation for 15 min. Methyl-2.5-dibromopentanoate (100 mM) was then added, and the reaction proceeded for 3 h with agitation, after which full dehydrothiolation was observed.⁸ The reaction was centrifuged for 5 min at 17.000×g, and the supernatant was subsequently collected to remove precipitated MBP from the mixture. Six times the reaction volume of diethyl ether was added, and the mixture was vortexed for 10 sec. The mixture was then centrifuged for 15 s at 6,000×g before removing the top ether layer. This wash was repeated a second time before the tubes were incubated at 37° C. for 10 min with the cap open to remove excess diethyl ether. The dehydrothiolated substrates were then dried by a SpeedVac Vacuum concentrator (Thermo Scientific) and resuspended in 50 mM HEPES pH 7.5 prior to the [4+2] cyclization assay.

### Example 20. In Vitro Translation and Thiazol(in)es-Containing Pyritide Biosynthesis by TbtE/F/G and MroB/C/D

The in vitro translation was performed as mentioned above in a 15 μL scale reaction. After the substrate was generated, the mixture was generated into two parts: 5 and 10 μL part. The 10 μL part was incubated with 2 μM MBP-TbtE (with no TEV cleavage site). 2 μM MBP-TbtF. 2 μM MBP-TbtG. 20 mM of MgCl₂, 6 mM ATP. 50 mM HEPES pH 7.5, and TEV (L56V/S135G/S219V) (w/w ratio with total amount of MBP-tagged protein=1:7) for 18 h at room temperature in a 20 μL reaction. MBP-TbtF and MBP-TbtG contain a TEV cleavage site (ENLYFQS: SEQ ID NO:281) between MBP and the protein of interest. The 5 μL part was incubated with the same mixture but did not contain any enzymes as a negative control. 2.5 out of 5 μL was incubated with 5 mM IAA for 1 h at room temperature.

The 20 μL Tbt/E/F/G reaction was then divided equally into two parts. The first half (10) μL) was incubated with 2 μM MBP-MroB. 2 μM MBP-MroC. 6 mM ATP. 50 mM HEPES pH 7.5, 1 μM of T. bispora GluRS. L-glutamate 1 mM, 3 μM T. bispora tRNA^{Glu} (CUC), 0.027 U μL-1 TIPP, and 5 mM IAA for 1.5 h at room temperature in a 30 μL reaction. In the second half (10 μL), 5 μL was incubated with the same mixture but did not contain any enzymes and tRNA as a negative control. The remaining 5 μL underwent a similar incubation process but without any IAA.

The 30-μL reaction MroB/C reaction was then divided equally into two parts. One part was treated with MBP-MroD such that the final concentration of MBP-MroD is 3 μM, and the other part was added the same volume but with buffer lacking MBP-MroD (50 mM HEPES, 300 mM NaCl, 2.5% glycerol, 0.5 mM TCEP). The reaction further proceeded for 1.5 h at room temperature. All reaction mixture was then desalted with solid-phase extraction using Ziptip and analyzed with MALDI-TOF-MS as mentioned above.

For LC-HR-ESI-MS analysis, a 30 μL scale in vitro translation reaction was performed and incubated with Tbt/E/F/G and MroB/C/D as mentioned above but without any splitting, resulting in a 185 μL reaction after adding all enzymes and necessary components. This mixture was then desalted with solid-phase extraction using an 8 mg Pierce C18 Spin Column (Thermo-Fisher Scientific). The desalting protocol was performed following the manufacturer's instructions but omitted TFA. The eluant (in 80% acetonitrile) was centrifuged at 13,000×g. Then, the supernatant was collected and directly injected onto LC-MS.

### Example 21. LC-HR-ESI-MS/MS Analysis of Dehydration and Cyclization Assays

Enzymatic assays were desalted using solid-phase extraction prior to LC-ESI-MS/MS analysis. Specifically, except for the thiazol(in)es-containing pyritide (which utilize 8 mg Pierce C18 Spin Column), the samples were applied to Toptip C18 (10-200 μL, Glygen Corp) wetted with 50 μL of 80% acetonitrile (0.1% formic acid) and equilibrate with 150 μL of 0.1% formic acid following the manufacturer instructions. The C18 columns (tips) are then washed with 150 μL of 0.1% formic acid and eluted using 100 μL 80% acetonitrile (0.1% formic acid). The samples were then dried utilizing lyophilization and redissolved in 25% acetonitrile (80 μL for enzymatic assays of in vitro-translated substrates and 200 μL for enzymatic assays of purified substrates). Then 20 μL of each sample was injected into an Agilent AdvanceBio Peptide Plus column (2.1×150 mm, 2.7 μm) equipped with an Agilent 6545B Q-TOF interfaced with an Agilent 1290 Infinity II LC system. Mobile phase solvents were composed of H₂O, 0.1% formic acid (Solvent A), and acetonitrile 0.1% formic acid (Solvent B). The column compartment was maintained at 35° C. during all experiments. The column was equilibrated with 5 column volumes of starting mobile phase (95% A and 5% B) between injections. The gradient of all LC runs was as follows: 0-2 min: 95% A 5% B, 2-3 min: 70% A 30% B, 3-18 min: 20% A 80% B, 18-20 min: 5% A 95% B. The samples were run to waste for the first 3 min before applying to the mass spectrometer. Mass range was set from 100 to 1700 m/z (except for 412MroA1 W7G: 100-3000 m/z). MS parameters were as follows: gas, 320° C. at 8 L/min: nebulizer, 35 psig: nozzle voltage, 1000 V: sheath gas, 350° C. at 11 L/min: capillary, 3500 V: fragmentor, 125 V: skimmer, 65 V: MS scan rate (10) spectra/s): MS-MS scan rate (5 spectra/s); and isolation width (MS/MS), 1.3 m z. The MS was operated in positive ionization mode for all samples analyzed, and fragmentation was performed using collision-induced dissociation (CID) at 25 eV. For the thiazol(in)es-containing pyritides, the nozzle voltage utilized was 0) V. Data analysis was conducted using Agilent MassHunter Qualitative Analysis 10.0. The exact mass lists are exported and analyzed using IPSA⁹ and mMass.¹⁰

For Thr-containing substrates, in vitro translation reactions (12.5 μL) were performed followed by MroB/C/D assays as mentioned above and GluC (NEB) digestion (500 ng) in NEB's GluC Reaction Buffer for 12 h at room temperature. These mixtures were then desalted using solid-phase extraction with Toptip C18 as mentioned above prior to LC-MS/MS analysis. Fragmentation was performed as mentioned above using CID at 22 or 25 eV. For GluC-digested dehydrated MroA1 G5T (VGADhaWLTDhaWVI: SEQ ID NO:283), fragmentation was performed using CID at 10 eV instead.

### Example 22. HR-ESI-MS/MS (Non-LC) Analysis of Dehydration and Cyclization Assays

MroB/C and MroB/C/D assays of triArg-MroA1 and triArg-MroA2 were analyzed with high-resolution tandem-mass spectrometry without liquid chromatography on a ThermoFisher Scientific Orbitrap Fusion ESI-MS using an Advion TriVersa Nanomate 100. The assays were desalted using C18 Ziptip (EMD Millipore) and eluted using 80% acetonitrile with 1% acetic acid. The MS was calibrated and tuned with Pierce LTQ Velos ESI Positive Ion Calibration Solution (ThermoFisher). Samples were directly infused into a ThermoFisher Scientific Orbitrap Fusion ESI-MS using an Advion TriVersa Nanomate 100. The MS was calibrated and tuned with Pierce LTQ Velos ESI Positive Ion Calibration Solution (ThermoFisher). The MS was operated using the following parameters: mass range, 100-2000 m/z; resolution, 120,000; isolation width (MS/MS), 1 m/z; normalized collision energy (MS/MS), 30 (didehydrated MroA1, MroA1 and MroA2 ejected leader peptide) or 70 (didehydrated MroA2); activation q value (MS/MS), 0.4; activation time (MS/MS), 30 ms. Fragmentation was performed using collision-induced dissociation (CID) at 30% or 70%. Data analysis was conducted using the Qualbrowser application of Xcalibur software (Thermo-Fisher Scientific). The exact mass lists are exported and analyzed using IPSA⁹ and mMass.¹⁰

### Example 23. Solid-Phase Peptide Synthesis (SPPS) Protocol of Gly-Ala-MroA1 Core Peptide

Manual fluorenylmethyloxycarbonyl (Fmoc) SPPS was performed at room temperature using a 25 mL fritted glass funnel as a reaction vessel, dimethylformamide (DMF) as a solvent, 2-(6-Chloro-1-H-benzotriazole-1-yl)-1,1,3,3-tetramethylaminium hexafluorophosphate (HCTU) as an activator, 20:80 N-methylmorpholine:DMF as coupling solution, 20:80 piperidine:DMF as deprotection solution, and 60:40 acetic anhydride:pyridine as a capping solution. The peptide was synthesized on a 0.05 mmol scale starting from the Fmoc-Ile Wang resin. The resin was bubbled twice with 5 mL of deprotection solution for each coupling cycle, followed by washing five times with DMF. Next, a 5-molar equivalence of Fmoc-amino acid and HCTU was dissolved in a 5-mL coupling solution and added to the resin. Coupling was performed for 15-20 min, followed by washing 5 times with DMF. After the last amino acid was coupled, the peptide was deprotected and capped with 5 mL of capping solution for 30 min. Finally, the resin was washed with DMF and dichloromethane then dried under vacuum. For global deprotection and cleavage from the linker, the resin was resuspended in 5 mL of deprotection solution (TFA:Triisopropylsilane:H₂O 95:2.5:2.5) for 2 h at room temperature. The solution was filtered by passing through a glass wool-packed pipet, then gently dried under nitrogen to ˜1 mL final volume and added dropwise to 10 mL of ice-cold diethyl ether to precipitate the peptide. The precipitate was collected by centrifugation, dissolved in ˜5 mL of DMF, and further purified by RP-HPLC (Shimadzu LC system) using the following condition:

TABLE 4

| | |
|---|---|
| Column | Macherey Nagel-C18 HTec, 250 × 10 mm, 5 μm |
| Solvent | A: 0.1% TFA in H₂O |
| | B: 0.1% TFA in acetonitrile |
| Gradient | 1 mL injection, 4 mL/min |
| | 0-15 min: 2-30% B |
| | 15-45 min: 30-60% B |

Under these conditions, the MroA1 core peptide elutes around 32-34 min.

### Example 24. Protocol to Produce Fluorescein-Labeled Δ12MroA1

HPLC-purified Δ12MroA1 was dissolved in 50 μL of 100 mM sodium borate pH 8.4 to 0.5-2 mM. To this solution, 50 μL of 5/6-carboxyfluorescein succinimidyl ester (Thermo Fisher) in DMF (10 mg/mL) was slowly added. The reaction was quickly mixed and protected from light. After 2-4 h, the reaction progress was checked with MALDI-TOF MS (successful labeling was indicated by an +358 adduct). The reaction was diluted 10-fold with 100 mM Tris pH 8, then subjected to centrifugation to remove insoluble materials. The supernatant was injected onto the RP-HPLC Phenomenex Luna C5 column (250-10 cm, 100 Angstrom, 5 microns) connected to an HPLC system (Shimadzu) running at 4 mL/min of solvent A (H₂O+20 mM ammonium acetate) and solvent B (acetonitrile). The following gradient was used: 0-15 min: 2-30% B. 15-45 min: 30-60% B. HPLC fractions were monitored by MALDI-TOF MS (Bruker Ultraflex). Labeled peptide elutes around 22-25 min. These fractions were collected, protected from light, and lyophilized to dryness.

### Example 25. Fluorescence Polarization to Measure K_{D} of MroA1 with MroB or MroD

All proteins and peptides were prepared in the 0.5× storage buffer before concentration/FP measurement. Experiments were done in triplicates. Stock fluorescein-labeled peptide was measured concentration using A₄₉₀ (ε: 70,000 M⁻¹ cm⁻¹). Initial sample was prepared: 5 nM labelled Δ12MroA1 (for MroD; for MroB, only 2 nM labelled Δ12MroA1 was used), 10 μM MBP-MroB (A₂₈₀ ε: 178,885 M⁻¹ cm⁻¹) or 10 μM MBP-MroD (A₂₈₀ ε:115,740 M⁻¹ cm⁻¹). In a black 96-well plate (Corning 3686), 50 μL of initial sample was added to the first well, followed by 11 3-fold dilutions into subsequent wells containing 5 nM labeled Δ12MroA1. The plate was covered from light and incubated at room temperature for 1 h, and then fluorescence polarization was measured (Biotek Synergy H4 hybrid reader) using the following filter (Excitation: Emission—485 nm/20 nm: 518 nm/20 nm). The obtained data was converted to anisotropy value and plotted against protein concentration. Using the OriginPro software, the data were fitted to receptor depletion equation:¹¹

$$y = A 1 + ( A 2 - A 1 ) \frac{( Lt + Kd + x ) - \sqrt{{( Lt + Kd + x )}^{2} - 4 * Lt * x}}{2 Lt}$$

Where: y=anisotropy value, A1=minimum anisotropy, A2=maximum anisotropy, Lt=probe concentration, and x=total enzyme concentration.

### Example 26. Competition Fluorescence Polarization

The initial sample contained 80 nM enzyme, 5 nM labeled Δ12MroA1 peptide, and competitor peptide (concentration is from 20 μM to 100 μM, depending on the experiment). In a black 384-well plate (Corning 3575), 30 μL of initial sample was added to the first well, followed by 13 2-fold dilutions into subsequent wells containing 80 nM MBP-MroB and 5 nM labeled Δ12MroA1. The plate was covered from light and incubated at room temperature for 10 min, and then fluorescence polarization was measured (Biotek Synergy H4 hybrid reader) using the following filter (Ex: Em—485 nm/20 nm: 518 nm/20 nm). The obtained data was converted to anisotropy value and plotted against peptide concentration, and fitted to dose-response function:

$$y = A 1 + \frac{( A 2 - A 1 )}{1 + {( \frac{IC 50}{x} )}^{p}}$$

Where: y=anisotropy value, A1=minimum anisotropy, A2=maximum anisotropy, p=Hill's coefficient, x=competitor concentration. The obtained IC₅₀ was used to calculate inhibitor constant Krusing the following equation: 12

$$Ki = \frac{IC 50}{1 + \frac{Lt ( y + 2 )}{2 * Kd ( y + 1 )}} - Kd ( \frac{y}{y + 2} )$$

Where: Lt is labeled peptide concentration, y=initial bound/free ratio of the labeled peptide before adding competitor, and Kd is the binding constant.

In cases where competitor concentration was not high enough to achieve a plateau, the competition curve was fitted with minimum anisotropy fixed to be the same value as the anisotropy value of the free probe solution. This calculation assumed that the labeled peptide was completely displaced from the enzyme binding pocket at a very high concentration of the competitor. Hence, the anisotropy value was similar to the sample with only the labeled peptide. The specific minimum anisotropy used in this experiment for both MroB and MroD was 65.

TABLE 5
Binding of MroA1 Variants to MroB and MroD. Ac = N-acetyl.

| | MroA1 | | IC 50 MroB | IC50 MroD |
|---|---|---|---|---|
| SEQ ID NO | Variants | Sequence | (μM) | (μM) |
| 55 | Δ12MroA1 | SDLDIVDLDLAVDEELAALSVGGLGNTE | 0.68 ± 0.04 | 0.9 ± 0.02 |
| | | VGASGWLGSWVI | | |
| 159 | Δ12MroA1 | SDLDIVDLDLAVDEELAALSVGGLGNTE | 19.4 ± 1.6 | 16.0 ± 4.4 |
| | Leader | VGA | | |
| 158 | GlyAla- | Ac-GASGWLGSWVI | 27.2 ± 1.9 | 40.9 ± 5.0 |
| | MroA1 core | | | |
| 160 | Δ12MroA1- | SDLDIVDLDLAVDEELAALSVGGLGNTE | 8.1 ± 3.9 | 2.4 ± 0.3 |
| | W7G | VGASGWLGSGVI | | |
| 161 | Δ12MroA1- | SDLDIVDLDLAVDEELAALSVGGLGNTE | 1.1 ± 0.2 | 1.7 ± 0.4 |
| | V8G | VGASGWLGSWGI | | |
| 162 | Δ12MroA1- | SDLDIVDLDLAVDEELAALSVGGLGNTE | 2.0 ± 0.6 | 0.91 ± 0.09 |
| | I9G | VGASGWLGSWVG | | |
| 55 | Δ12MroA1- | SDLDIVDLDLAVDEELAALSVGGLGNTE | 4.2 ± 1.7 | 0.40 ± 0.06 |
| | COOMe | VGASGWLGSWVI-COOMe | | |

TABLE 6
Amino Acid Abbreviations
Amino Acid Abbreviations

| | | |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |
| Dehydroalanine | Dha | |
| Dehydrobutyrine | Dhb | |

### Example 27 RGD-Binding Integrin Avb3

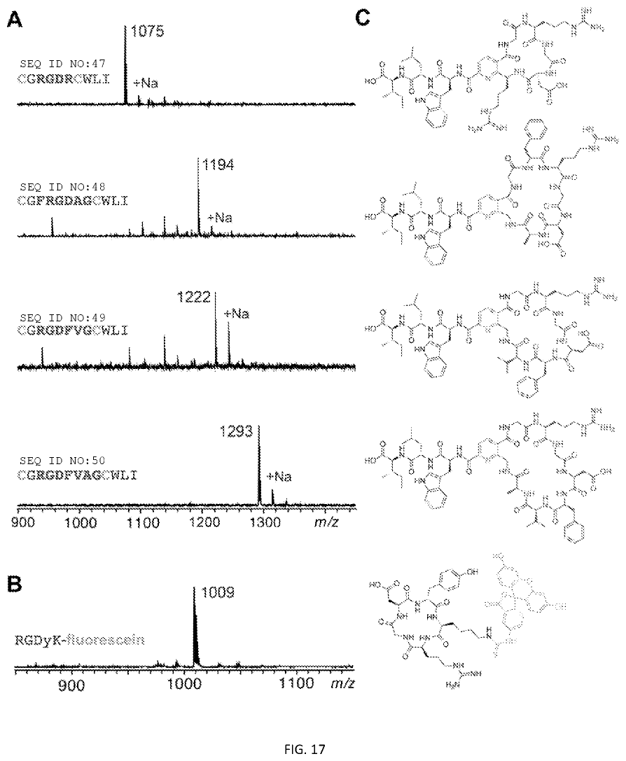

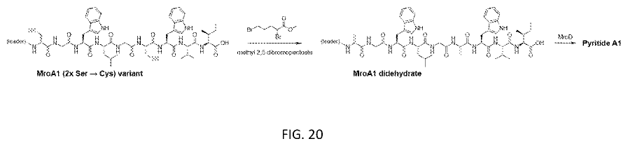

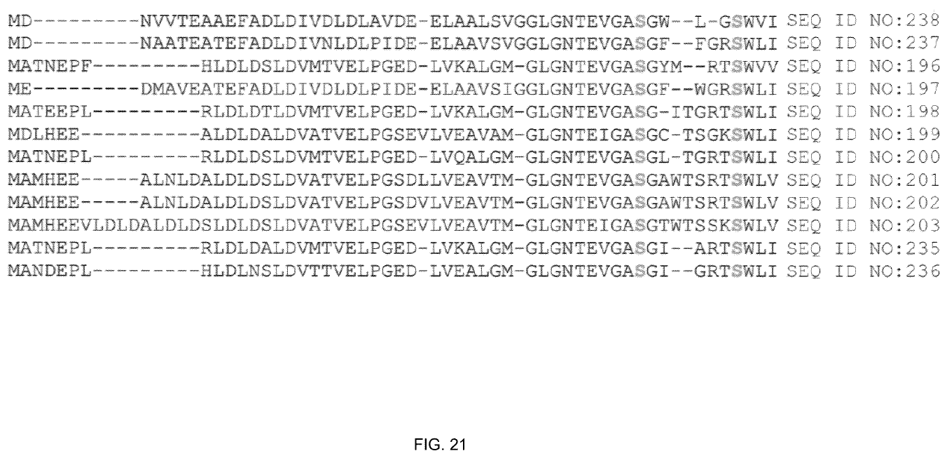

Several pyritide precursor peptides were designed with various permutations of RGD-binding integrin avb3 in the core region (currently all in the L-stereochemical configuration, FIG. 17). To evaluate if these RDG epitope grafts were compatible with pyridine synthase MroD, we cloned, expressed, and purified the precursor peptides as C-terminal fusions to maltose-binding protein (MBP) in E. coli. The MroD substrates were prepared using the chemoenzymatic route depicted (FIG. 20). Dehydrothiolation reagents and byproducts are removed by ether extraction prior to treatment with recombinantly expressed and purified MBP-MroD (3 h at 37° C.). The samples were then desalted with C18 ZipTips and subjected to MALDI-TOF mass spectrometry without further purification. All four RGD-grafted pyritides were successfully converted into the expected pyritide, regardless of the RGD start position (2^{nd} or 3^{rd}), identify of residues flanking the RGD motif, and macrocycle size (5 to 8 residues total, FIG. 17). These compounds are now ready for a preparative-scale production followed by HPLC purification.

We have synthesized FITC-labeled cyclic RGDyK (v. D-Tyr) and this peptide will be HPLC purified to allow for competition fluorescence polarization (FP) studies against commercially available integrins aᵥb3, aᵥb5, and a_{IIb}b3. ELISA will also be used to quantify any interactions measured to be tighter than ˜25 nM.

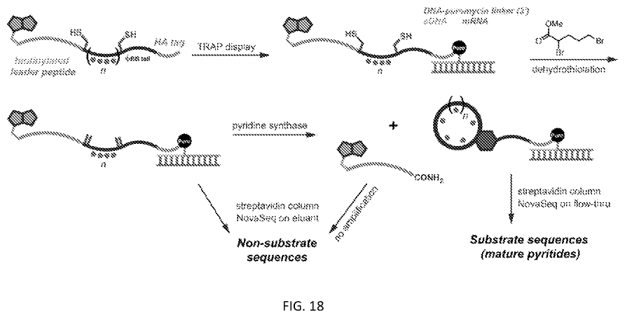

Substrate tolerance (MroD only). The Mro biosynthetic pathway (FIG. 3 and FIG. 17) has good tolerance for substrates. The pathway has high tolerance towards sequence variation in the ring while the tail tripeptide region is much more restrictive. The lower tolerance of MroB/C may impact the availability of substrates for MroD (fully enzymatic versus dehydrothiolation “bypass” methods shown in FIG. 20). Further, the size ranges and possible epistatic interactions noted in other RiPP pathways may complicate outcomes. For these reasons, we will use the WLI tail primarily as a tether for transcription-translation coupled with association of puromycin linker (TRAP) display, an improvement over standard mRNA display methodology. The overall TRAP display experiment to evaluate MroD substrate tolerance (i.e., using chemical dehydrothiolation to bypass MroB/C) on a broad scale is depicted in FIG. 18.

As illustrated, the proposed pyritide TRAP display procedure is highly modular (FIG. 18). After chemical dehydrithiolation and MroD-catalyzed pyridine formation, any unreacted precursor peptides and the leader region of processed substrates are removed by streptavidin affinity chromatography. As an alternative, any orthogonal N-terminal affinity tag will suffice for this separation, including His6 with Ni-NTA-based removal. The dots represent the location for variation within the library, which will be generated from five parallel constructs. The first has a core sequence of: CGX₃CWLI (SEQ ID NO:277), where X is any of the 20 common proteinogenic amino acids for a library size of 203=8 k unique sequences (encoded by the NNK codon). The next four constructs each expand the “X” region by one amino acid, thus consecutively increasing macrocycle size by one residue for library sizes of: 20⁴=160 k: 20⁵=3.2M: 20⁶=64M; and 20⁷=1.28B (total unique sequences ˜1.35B). The substrate and non-substrate cohorts will be subjected to NovaSeq 6000 sequencing using the S1 flow cell. Acquiring reads in the 2×150 bp format should give 400-500 Gb of data, which allows for a confident read-depth for this experiment and the analysis described below. Standard data processing and bioinformatic workflows will be used to pattern match any discernible preferences of substrate versus non-substrate sequences.

Approximately 20% of the sequences will encode a premature stop codon since each usage of NNK introduces a 1 in 32 probability of a premature stop codon. We do not view this as a problem since none of those sequences will contain an amplification handle and thus cannot be amplified or sequenced. Another 1 in 32 per NNK codon probability we will encounter is where additional cysteine residues are introduced. These will be chemically dehydrothiolated to Dha and could result in a mixture of isobaric products with variable ring sizes. Based on earlier work, the positioning of the C-terminal Dha to WLI appears important, so we are led to believe that the desired macrocycle will be kinetically preferred; nevertheless, we will address potential isobaric outcomes in later characterization steps.

While the workflow presents substrates and non-substrates as a “binary”, we expect these bins to partially overlap. The level should correlate with MroD efficiency and duration of treatment. We expect efficient MroD substrates will be underrepresented in the non-substrate samples. The inverse should also be true (i.e., inefficient MroD substrates should be overrepresented in the non-substrate bin). The ratio of read depths may further indicate how “good” a particular MroD substrate is, providing a spectrum of tolerability rather than a qualitative yes/no. This is the principal justification for using the S1 flow cell over the SP. If necessary, S2 or S4 flow cells can be used, although these are associated with substantial cost increases. This level of resolution on substrate tolerance across a dataset this large is not available for any RiPP. Such data will become increasingly important as AI algorithms are applied to predict the outcomes for RiPP biosynthetic pathway tolerances. As such, we will test these features with several positive controls that robustly yield the expected pyritide and several negative controls that do not form pyritides under any condition. Intermediate level substrates will also be assessed to determining if read depth ratios carry quantitative rather than qualitative information about substrate tolerance.

Substrate tolerance (MroB/C/D). The experimental plan of described above (Substrate tolerance (MroD only) will be modified to assess the substrate tolerance for the fully enzymatic production of pyritides where MroB/C are used to afford the didehydrated peptide substrate for MroD. The library constructs had core sequences of CGX₃₋₇CWLI (SEQ ID NO: 278), which now will be replaced with SGX₃₋₇SWLI (SEQ ID NO:279) (total of ˜1.35B sequences). While the stop codon issue will remain the same as described in the section. Substrate tolerance (MroD only), situations when X=cysteine will not pose an issue since MroB/C are specific for serine.⁴ As above. NovaSeq 6000 using the S1 flow cell will be used to sequence the substrate and non-substrate cohorts. Pattern matching will proceed as above but with an additional layer of scrutiny to assess the impact of chemical versus enzymatic dehydrothiolation on pathway scope.

Substrate validation. To corroborate the sequencing results from the Substrate tolerance (MroD only) and Substrate tolerance (MroB/C/D) sections (chemoenzymatic and fully enzymatic routes, respectively), and to apply a high level of rigor in accord with our past practices²⁹, we will prepare gene constructs for 20 randomly chosen pyritide precursor peptides from the Substrate tolerance (MroD only) and Substrate tolerance (MroB/C/D) substrate cohorts. A second set of 20 pyritide precursor peptides will be randomly selected from the Substrate tolerance (MroD only) and Substrate tolerance (MroB/C/D) sections non-substrate cohorts. These 80 peptides will be expressed using cell-free biosynthesis methods and treated with MroD (4.1 cohort) or MroB/C/D (4.2 cohort) and analyzed by MALDI-TOF mass spectrometry (MS). Further characterization of the product-forming sequences will be assessed through high-resolution and tandem MS (HRMS/MS) using an ThermoFisher Q-Exactive orbitrap instrument. We anticipate a high level of agreement with the NovaSeq results, assuming read-depth is adequate across the set.

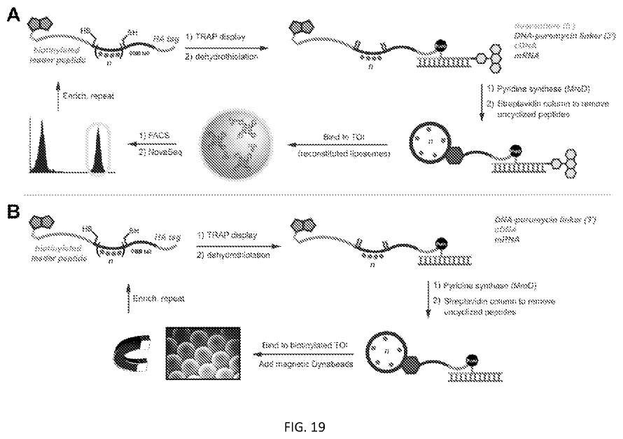

Selection of integrin binders. In a manner analogous to that shown in Substrate tolerance (MroD only) section, the TRAP display method is modified, this time with a 5′-fluorophore labeled DNA oligonucleotide (FIG. 19A). Formation of the pyritide-TRAP-fluorophore conjugates will either proceed by using MroB/C/D or the chemical dehydrothiolation “bypass” method. Separately, the desired integrin (starting with aᵥb3) will be reconstituted in liposomes with ˜250 nm diameters. Empty liposomes of the same dimensions will also be prepared. Any pyritide-TRAP-fluorophore conjugates that bind to empty liposomes will be removed in a first round of negative selection using fluorescence activated cell sorting (FACS). Non-binding pyritide-TRAP-fluorophore conjugates are far too small to be “sorted” by FACS. After amplification of the non-binders by PCR, a second round of selection will allow binding of the “pre-cleared” pyritide-TRAP-fluorophore conjugates to integrin aᵥb3-loaded liposomes under generous binding conditions. Pyritides with respectable affinity to the integrin will be positively selected by FACS, and a preliminary analysis of the enriched sequences will be evaluated by NovaSeq. During subsequent rounds, binding stringency will be increased if the recovered nucleic acid and sequence diversity is judged to be sufficient; if insufficient, the binding stringency will be adjusted.

An alternative TRAP setup (FIG. 19B) replaces the fluorophore, liposomes, and FACS, with a biotinylated target of interest and magnetic streptavidin-functionalized beads.

The sequences encoding for pyritides that survive binding under the most stringent conditions will be assessed and the upwards of 10 will be prepared by in vitro biosynthetic methods. Binding affinity will be assessed against integrins aᵥb3, aᵥb5, and a_{IIb}b3 using RGDyK-fluorescein (FIG. 17) using a convenient, competitive FP assay. ELISA will be used for any compounds binding tighter than ˜25 nM.

Less than <10 rounds of selection will be necessary to obtain the desired pyritide binding activities. The synthesis of the tightest binder, if desired, can be scaled up to the mg scale for characterization by NMR spectroscopy. The compound can also be assessed for binding activity against any other desired integrins that are commercially available.

An alternative method to identify RGD binding molecules will be identified by preparing a library with SGX₀₋₃RGDX₀₋₃SWLI (SEQ ID NO:46) (the two serines may be cysteines, depending on which synthetic route is chosen). The total “RDG-biased” library contains 67,368 unique members. The selection method, analysis, scaleup and characterization will parallel that described above.

The relevant methods used are as described in Examples 2-26.

### DOCUMENTS CITED

(1) Bockus, A. T.; McEwen, C. M.; Lokey, R. S. Form and function in cyclic peptide natural products: a pharmacokinetic perspective Curr. Top. Med. Chem 2013, 13, 821.
(2) Choi, J. S.; Joo, S. H. Recent trends in cyclic peptides as therapeutic agents and biochemical tools Biomol. Ther. 2020, 28, 18.
(3) Nielsen, D. S.; Shepherd, N. E.; Xu, W.; Lucke, A. J.; Stoermer, M. J.; Fairlie, D. P. Orally absorbed cyclic peptides Chem. Rev 2017, 117, 8094.
(4) Vinogradov, A. A.; Yin, Y.; Suga, H. Macrocyclic peptides as drug candidates: recent progress and remaining challenges J. Am. Chem. Soc. 2019, 141, 4167.
(5) Sohrabi, C.; Foster, A.; Tavassoli, A. Methods for generating and screening libraries of genetically encoded cyclic peptides in drug discovery Nat. Rev. Chem. 2020, 4, 90.
(6) Wang, C. K.; Gruber, C. W.; Cemazar, M.; Siatskas, C.; Tagore, P.; Payne, N.; Sun, G.; Wang, S.; Bernard, C. C.; Craik, D. J. Molecular grafting onto a stable framework yields novel cyclic peptides for the treatment of multiple sclerosis ACS Chem. Biol. 2014, 9, 156.
(7) Veber, D. F.; Freidlinger, R. M.; Perlow, D. S.; Paleveda, W. J., Jr.; Holly, F. W.; Strachan, R. G.; Nutt, R. F.; Arison, B. H.; Homnick, C.; Randall, W. C.; Glitzer, M. S.; Saperstein, R.; Hirschmann, R. A potent cyclic hexapeptide analogue of somatostatin Nature 1981, 292, 55.
(8) Chow, H. Y.; Zhang, Y.; Matheson, E.; Li, X. Ligation technologies for the synthesis of cyclic peptides Chem. Rev 2019, 119, 9971.
(9) Reguera, L.; Rivera, D. G. Multicomponent reaction toolbox for peptide macrocyclization and stapling Chem. Rev 2019, 119, 9836.
(10) Liu, D.; Rubin, G. M.; Dhakal, D.; Chen, M.; Ding, Y. Biocatalytic synthesis of peptidic natural products and related analogues iScience 2021, 24, 102512.
(11) Montalbán-López, M.; Scott, T. A.; Ramesh, S.; Rahman, I. R.; van Heel, A. J.; Viel, J. H.; Bandarian, V.; Dittmann, E.; Genilloud, O.; Goto, Y.; Grande Burgos, M. J.; Hill, C.; Kim, S.; Koehnke, J.; Latham, J. A.; Link, A. J.; Martinez, B.; Nair, S. K.; Nicolet, Y.; Rebuffat, S.; Sahl, H.-G.; Sareen, D.; Schmidt, E. W.; Schmitt, L.; Severinov, K.; Süssmuth, R. D.; Truman, A. W.; Wang, H.; Weng, J.-K.; van Wezel, G. P.; Zhang, Q.; Zhong, J.; Piel, J.; Mitchell, D. A.; Kuipers, O. P.; van der Donk, W. A. New developments in RiPP discovery, enzymology and engineering Nat. Prod. Rep. 2021, 138, 130

(12) Schmitt, S.; Montalban-Lopez, M.; Peterhoff, D.; Deng, J.; Wagner, R.; Held, M.; Kuipers, O. P.; Panke, S. Analysis of modular bioengineered antimicrobial lanthipeptides at nanoliter scale Nat. Chem. Biol. 2019, 15, 437.
(13) Yang, X.; Lennard, K. R.; He, C.; Walker, M. C.; Ball, A. T.; Doigneaux, C.; Tavassoli, A.; van der Donk, W. A. A lanthipeptide library used to identify a protein-protein interaction inhibitor Nat. Chem. Biol. 2018, 14, 375.
(14) Hetrick, K. J.; Walker, M. C.; van der Donk, W. A. Development and application of yeast and phage display of diverse lanthipeptides ACS Cent. Sci. 2018, 4, 458.
(15) Urban, J. H.; Moosmeier, M. A.; Aumüller, T.; Thein, M.; Bosma, T.; Rink, R.; Groth, K.; Zulley, M.; Siegers, K.; Tissot, K.; Moll, G. N.; Prassler, J. Phage display and selection of lanthipeptides on the carboxy-terminus of the gene-3 minor coat protein Nat. Commun. 2017, 8, 1500.
(16) Reyna-Gonzalez, E.; Schmid, B.; Petras, D.; Süssmuth, R. D.; Dittmann, E. Leader peptide-free in vitro reconstitution of microviridin biosynthesis enables design of synthetic protease-targeted libraries Angew. Chem. Int. Ed. 2016, 55, 9398.
(17) Vinogradov, A. A.; Suga, H. Introduction to thiopeptides: biological activity, biosynthesis, and strategies for functional reprogramming Cell Chem. Biol. 2020, 27, 1032.
(18) Burkhart, B. J.; Schwalen, C.; Mann, G.; Naismith, J. H.; Mitchell, D. A. YcaO-dependent posttranslational amide activation: biosynthesis, structure, and function Chem. Rev. 2017, 117, 5389.
(19) Ortega, M. A.; Hao, Y.; Zhang, Q.; Walker, M. C.; van der Donk, W. A.; Nair, S. K. Structure and mechanism of the tRNA-dependent lantibiotic dehydratase NisB Nature 2015, 517, 509.
(20) Hudson, G. A.; Zhang, Z.; Tietz, J. I.; Mitchell, D. A.; van der Donk, W. A. In vitro biosynthesis of the core scaffold of the thiopeptide thiomuracin J. Am. Chem. Soc. 2015, 137, 16012.
(21) Wever, W. J.; Bogart, J. W.; Baccile, J. A.; Chan, A. N.; Schroeder, F. C.; Bowers, A. A. Chemoenzymatic synthesis of thiazolyl peptide natural products featuring an enzyme-catalyzed formal [4+2] cycloaddition J. Am. Chem. Soc. 2015, 137, 3494.
(22) Bowers, A. A.; Acker, M. G.; Koglin, A.; Walsh, C. T. Manipulation of thiocillin variants by prepeptide gene replacement: structure, conformation, and activity of heterocycle substitution mutants J. Am. Chem. Soc. 2010, 132, 7519.
(23) Luo, X.; Zambaldo, C.; Liu, T.; Zhang, Y.; Xuan, W.; Wang, C.; Reed, S. A.; Yang, P. Y.; Wang, R. E.; Javahishvili, T.; Schultz, P. G.; Young, T. S. Recombinant thiopeptides containing noncanonical amino acids Proc. Natl. Acad. Sci. USA 2016, 113, 3615.
(24) Zhang, Z.; Hudson, G. A.; Mahanta, N.; Tietz, J. I.; van der Donk, W. A.; Mitchell, D. A. Biosynthetic timing and substrate specificity for the thiopeptide thiomuracin J. Am. Chem. Soc. 2016, 138, 15511.
(25) Bogart, J. W.; Bowers, A. A. Thiopeptide pyridine synthase TbtD catalyzes an intermolecular formal aza-diels-alder reaction J. Am. Chem. Soc. 2019, 141, 1842.
(26) Wever, W. J.; Bogart, J. W.; Bowers, A. A. Identification of pyridine synthase recognition sequences allows a modular solid-phase route to thiopeptide variants J. Am. Chem. Soc. 2016, 138, 13461.
(27) Vinogradov, A. A.; Shimomura, M.; Goto, Y.; Ozaki, T.; Asamizu, S.; Sugai, Y.; Suga, H.; Onaka, H. Minimal lactazole scaffold for in vitro thiopeptide bioengineering Nat. Commun. 2020, 11, 2272.
(28) Hudson, G. A.; Hooper, A. R.; DiCaprio, A. J.; Sarlah, D.; Mitchell, D. A. Structure prediction and synthesis of pyridine-based macrocyclic peptide natural products Org. Lett. 2021, 23, 253.
(29) Hegemann, J. D.; Bobeica, S. C.; Walker, M. C.; Bothwell, I. R.; van der Donk, W. A. Assessing the flexibility of the prochlorosin 2.8 scaffold for bioengineering applications ACS Synth. Biol 2019, 8, 1204.
(30) Si, Y.; Kretsch, A. M.; Daigh, L. M.; Burk, M. J.; Mitchell, D. A. Cell-free biosynthesis to evaluate lasso peptide formation and enzyme-substrate tolerance J. Am. Chem. Soc. 2021, 143, 5917.
(31) Precord, T. W.; Mahanta, N.; Mitchell, D. A. Reconstitution and substrate specificity of the thioether-forming radical S-adenosylmethionine enzyme in freyrasin biosynthesis ACS Chem. Biol. 2019, 14, 1981.
(32) Ruffner, D. E.; Schmidt, E. W.; Heemstra, J. R. Assessing the combinatorial potential of the RiPP cyanobactin tru pathway ACS Synth. Biol. 2015, 4, 482.
(33) Houssen, W. E.; Bent, A. F.; McEwan, A. R.; Pieiller, N.; Tabudravu, J.; Koehnke, J.; Mann, G.; Adaba, R. I.; Thomas, L.; Hawas, U. W.; Liu, H.; Schwarz-Linek, U.; Smith, M. C.; Naismith, J. H.; Jaspars, M. An efficient method for the in vitro production of azol(in)e-based cyclic peptides Angew. Chem. Int. Ed. 2014, 53, 14171.
(34) Czekster, C. M.; Ludewig, H.; McMahon, S. A.; Naismith, J. H. Characterization of a dual function macrocyclase enables design and use of efficient macrocyclization substrates Nat. Commun. 2017, 8, 1045.
(35) Ludewig, H.; Czekster, C. M.; Queis, E.; Munday, E. S.; Arshad, M.; Synowsky, S. A.; Bent, A. F.; Naismith, J. H. Characterization of the fast and promiscuous macrocyclase from plant PCY1 enables the use of simple substrates ACS Chem. Biol. 2018, 13, 801.
(36) Nguyen, G. K.; Wang, S.; Qiu, Y.; Hemu, X.; Lian, Y.; Tam, J. P. Butelase 1 is an Asx-specific ligase enabling peptide macrocyclization and synthesis Nat. Chem. Biol. 2014, 10, 732.
(37) Ting, C. P.; Funk, M. A.; Halaby, S. L.; Zhang, Z.; Gonen, T.; van der Donk, W. A. Use of a scaffold peptide in the biosynthesis of amino acid-derived natural products Science 2019, 365,280.
(38) Shimizu, Y.; Inoue, A.; Tomari, Y.; Suzuki, T.; Yokogawa, T.; Nishikawa, K.; Ueda, T. Cell-free translation reconstituted with purified components Nat. Biotechnol. 2001, 19, 751.
(39) Vinogradov, A. A.; Shimomura, M.; Kano, N.; Goto, Y.; Onaka, H.; Suga, H. Promiscuous enzymes cooperate at the substrate level en route to lactazole A J. Am. Chem. Soc. 2020, 142, 13886.
(40) Zhao, R.; Goldstein, S. A. N. Tethered peptide toxins for ion channels Methods Enzymol. 2021, 654, 203.
(41) Gräwe, A.; Ranglack, J.; Weyrich, A.; Stein, V. iFLinkC: an iterative functional linker cloning strategy for the combinatorial assembly and recombination of linker peptides with functional domains Nucleic Acids Res. 2020, 48, e24.
(42) Mack, M.; Riethmüller, G.; Kufer, P. A small bispecific antibody construct expressed as a functional single-chain molecule with high tumor cell cytotoxicity Proc. Natl. Acad. Sci. U.

S. A. 1995, 92, 7021.
(43) Lee, H. S.; Shu, L.; De Pascalis, R.; Giuliano, M.; Zhu, M.; Padlan, E. A.; Hand, P. H.; Schlom, J.; Hong, H. J.; Kashmiri, S. V. Generation and characterization of a novel single-gene-encoded single-chain immunoglobulin molecule with antigen binding activity and effector functions Mol. Immunol. 1999, 36, 61.
(44) Oman, T. J.; Knerr, P. J.; Bindman, N. A.; Vélasquez, J. E.; van der Donk, W. A. An engineered lantibiotic synthetase that does not require a leader peptide on its substrate J. Am. Chem. Soc. 2012, 134, 6952.
(45) Vinogradov, A. A.; Nagano, M.; Goto, Y.; Suga, H. Site-specific nonenzymatic peptide S/O-glutamylation reveals the extent of substrate promiscuity in glutamate elimination domains J. Am. Chem. Soc. 2021, 143, 13358.
(46) Huo, L.; Zhao, X.; Acedo, J. Z.; Estrada, P.; Nair, S. K.; van der Donk, W. A. Characterization of a dehydratase and methyltransferase in the biosynthesis of ribosomally synthesized and post-translationally modified peptides in Lachnospiraceae ChemBioChem 2020, 21, 190.
47. Gibson, D. G.; Young, L.; Chuang, R. Y.; Venter, J. C.; Hutchison, C. A., 3rd; Smith, H. O., Enzymatic assembly of DNA molecules up to several hundred kilobases. Nat. Methods 2009, 6 (5), 343.
48. Hudson, G. A.; Hooper, A. R.; DiCaprio, A. J.; Sarlah, D.; Mitchell, D. A., Structure prediction and synthesis of pyridine-based macrocyclic peptide natural products. Org. Lett. 2021, 23 (2), 253.
49. Ferrer, M.; Chernikova, T. N.; Timmis, K. N.; Golyshin, P. N., Expression of a temperature-sensitive esterase in a novel chaperone-based Escherichia coli strain. Appl Environ. Microbiol 2004, 70 (8), 4499.
50. Joseph, R. E.; Andreotti, A. H., Bacterial expression and purification of interleukin-2 tyrosine kinase: single step separation of the chaperonin impurity. Protein. Expr. Purif 2008, 60 (2), 194.
51. Cabrita, L. D.; Gilis, D.; Robertson, A. L.; Dehouck, Y.; Rooman, M.; Bottomley, S. P., Enhancing the stability and solubility of TEV protease using in silico design. Protein Sci 2007, 16 (11), 2360.
52. Huo, L.; Zhao, X.; Acedo, J. Z.; Estrada, P.; Nair, S. K.; van der Donk, W. A., Characterization of a dehydratase and methyltransferase in the biosynthesis of ribosomally synthesized and post-translationally modified peptides in Lachnospiraceae. ChemBioChem 2020, 21 (1-2), 190.
53. Hudson, G. A.; Zhang, Z.; Tietz, J. I.; Mitchell, D. A.; van der Donk, W. A., In vitro biosynthesis of the core scaffold of the thiopeptide thiomuracin. J. Am. Chem. Soc. 2015, 137 (51), 16012.
54. Morrison, P. M.; Foley, P. J.; Warriner, S. L.; Webb, M. E., Chemical generation and modification of peptides containing multiple dehydroalanines. Chem. Commun. 2015, 51 (70), 13470.
55. Brademan, D. R.; Riley, N. M.; Kwiecien, N. W.; Coon, J. J., Interactive peptide spectral annotator: A versatile web-based tool for proteomic applications*. Mol. Cell. Proteom. 2019, 18 (8, Supplement 1), S193.
56. Niedermeyer, T. H.; Strohalm, M., mMass as a software tool for the annotation of cyclic peptide tandem mass spectra. PLOS One 2012, 7 (9), e44913.
57. Lundblad, J. R.; Laurance, M.; Goodman, R. H., Fluorescence polarization analysis of protein-DNA and protein-protein interactions. J. Mol. Endocrinol. 1996, 10 (6), 607.
58. Munson, P. J.; Rodbard, D., An exact correction to the “cheng-prusoff” correction. J. Recept. Res. 1988, 8 (1-4), 533.
59. Paizs, B.; Suhai, S., Fragmentation pathways of protonated peptides. Mass Spectrom. Rev 2005, 24 (4), 508.
60. Kaufmann, R.; Kirsch, D.; Spengler, B., Sequenching of peptides in a time-of-flight mass spectrometer: evaluation of postsource decay following matrix-assisted laser desorption ionisation (MALDI). Int. J. Mass Spectrom. Ion Processes 1994, 131, 355.
61 Cohen, S. L., Ozone in ambient air as a source of adventitious oxidation. a mass spectrometric study. Anal. Chem. 2006, 78 (13), 4352.
62. Zhang, Z.; Hudson, G. A.; Mahanta, N.; Tietz, J. I.; van der Donk, W. A.; Mitchell, D. A., Biosynthetic timing and substrate specificity for the thiopeptide thiomuracin. J. Am. Chem. Soc. 2016, 138 (48), 15511.
63. Hudson, G. A., Hooper, A. R., DiCaprio, A. J., Sarlah, D. & Mitchell, D. A. Structure Prediction and Synthesis of Pyridine-Based Macrocyclic Peptide Natural Products. Org. Lett.
23, 253-256 (2021).
64. Montalbán-López. M. et al. New developments in RiPP discovery, enzymology and engineering. Nat. Prod. Rep. 38, 130-239 (2021).
65. Vitaku, E., Smith, D. T. & Njardarson, J. T. Analysis of the Structural Diversity. Substitution Patterns, and Frequency of Nitrogen Heterocycles among U.S. FDA Approved Pharmaceuticals. J. Med. Chem. 57, 10257-10274 (2014).
66. Nguyen, D. T. et al. Accessing Diverse Pyridine-Based Macrocyclic Peptides by a Two-Site Recognition Pathway. J. Am. Chem. Soc. 144, 11263-11269 (2022).
67. Ishizawa, T., Kawakami, T., Reid, P. C. & Murakami, H. TRAP Display: A High-Speed Selection Method for the Generation of Functional Polypeptides. J. Am. Chem. Soc. 135, 5433-5440) (2013).
68. Mas-Moruno, C., Rechenmacher, F. & Kessler, H. Cilengitide: The First Anti-Angiogenic Small Molecule Drug Candidate, Design, Synthesis and Clinical Evaluation. Anti-Cancer Agents Med. Chem. 10, 753-768.
69. Ongpipattanakul, C. et al. Mechanism of Action of Ribosomally Synthesized and Post-Translationally Modified Peptides. Chem. Rev. 122, 14722-14814 (2022).
70. Burkhart, B. J., Schwalen, C. J., Mann, G., Naismith, J. H. & Mitchell. D. A. YcaO-Dependent Posttranslational Amide Activation: Biosynthesis, Structure, and Function. Chem. Rev. 117, 5389-5456 (2017).
71. Schwalen, C. J., Hudson, G. A., Kille, B. & Mitchell. D. A. Bioinformatic Expansion and Discovery of Thiopeptide Antibiotics. J. Am. Chem. Soc. 140, 9494-9501 (2018).
72. Tietz, J. I. et al. A new genome-mining tool redefines the lasso peptide biosynthetic landscape. Nat. Chem. Biol. 13, 470-478 (2017).
73. Melby, J. O., Nard, N. J. & Mitchell, D. A. Thiazole/oxazole-modified microcins: complex natural products from ribosomal templates. Curr. Op. Chem. Biol. 15, 369-78 (2011).
74. Hudson, G. A. & Mitchell, D. A. RiPP antibiotics: biosynthesis and engineering potential. Curr. Op. Microbiol. 45, 61-69 (2018).
75. Burkhart, B. J., Kakkar, N., Hudson, G. A., van der Donk, W. A. & Mitchell, D. A. Chimeric Leader Peptides for the Generation of Non-Natural Hybrid RiPP Products. ACS Cent. Sci. 3, 629-638 (2017).
76. Hudson, G. A., Zhang, Z., Tietz, J. I., Mitchell, D. A. & van der Donk, W. A. In Vitro Biosynthesis of the Core Scaffold of the Thiopeptide Thiomuracin. J. Am. Chem. Soc. 137, 16012-16015 (2015).
77. Zhang, Z. et al. Biosynthetic Timing and Substrate Specificity for the Thiopeptide Thiomuracin. J. Am. Chem. Soc. 138, 15511-15514 (2016).
78. Cogan. D. P. et al. Structural insights into enzymatic [4+2] aza-cycloaddition in thiopeptide antibiotic biosynthesis. Proc. Natl. Acad. Sei. USA 114, 12928-12933 (2017).
79. Mahanta, N., Zhang, Z., Hudson, G. A., van der Donk, W. A. & Mitchell, D. A. Reconstitution and Substrate Specificity of the Radical S-Adenosyl-methionine Thiazole C-Methyltransferase in Thiomuracin Biosynthesis. J. Am. Chem. Soc. 139, 4310-4313 (2017).
80. Rice, A. J. et al. Enzymatic Pyridine Aromatization during Thiopeptide Biosynthesis. J. Am. Chem. Soc. 144, 21116-21124 (2022).
81. Burkhart, B. J., Hudson, G. A., Dunbar, K. L. & Mitchell, D. A. A prevalent peptide-binding domain guides ribosomal natural product biosynthesis. Nat. Chem. Biol. 11, 564-570 (2015).
82. Ortega, M. A. et al. Structure and mechanism of the tRNA-dependent lantibiotic dehydratase NisB. Nature 517, 509-512 (2015).
83. Morrison, P. M., Foley, P. J., Warriner, S. L. & Webb, M. E. Chemical generation and modification of peptides containing multiple dehydroalanines. Chem. Commun. 51, 13470-13473 (2015).
84. Ludwig, B. S., Kessler, H., Kossatz, S. & Reuning, U. RGD-Binding Integrins Revisited: How Recently Discovered Functions and Novel Synthetic Ligands (Re-)Shape an Ever-Evolving Field. Cancers 13, 1711 (2021).
85. Knappe, T. A. et al. Introducing lasso peptides as molecular scaffolds for drug design: Engineering of an integrin antagonist. Angew. Chem. Int. Ed. 50, 8714-7 (2011).
24. Hegemann, J. D. et al. Rational Improvement of the Affinity and Selectivity of Integrin Binding of Grafted Lasso Peptides. J. Med. Chem. 57, 5829-5834 (2014).
86. Hruby, V. J. Conformational restrictions of biologically active peptides via amino acid side chain groups. Life Sci. 31, 189-199 (1982).
87. Kessler, H. Conformation and Biological Activity of Cyclic Peptides. Angew. Chem. Int. Ed. 21, 512-523 (1982).
88. Eisele, G. et al. Cilengitide treatment of newly diagnosed glioblastoma patients does not alter patterns of progression. J. Neurooncol. 117, 141-145 (2014).
89. Vinogradov, A. A., Chang, J. S., Onaka, H., Goto, Y. & Suga, H. Accurate Models of Substrate Preferences of Post-Translational Modification Enzymes from a Combination of mRNA Display and Deep Learning. ACS Cent. Sci. 8, 814-824 (2022).
90. Si, Y., Kretsch, A. M., Daigh, L. M., Burk, M. J. & Mitchell, D. A. Cell-Free Biosynthesis to Evaluate Lasso Peptide Formation and Enzyme-Substrate Tolerance. J. Am. Chem. Soc. 143, 5917-5927 (2021).
91. Kondo, T. et al. cDNA TRAP display for rapid and stable in vitro selection of antibody-like proteins. Chem. Commun. 57, 2416-2419 (2021).
92. Brousseau, M. E. et al. Identification of a PCSK9-LDLR disruptor peptide with in vivo function. Cell Chem. Biol. 29, 249-258.e5 (2022).

1. A substrate for enzyme synthesis of pyridine-based macrocyclic peptides comprising a leader region and a core region, wherein the leader region comprises:
(SEQ ID NO: 1)
X₁LDX₂X₃X₄X₅X₆LX₇X₈X₉X₁₀X₁₁LX₁₂X₁₃X₁₄X₁₅X₁₆X₁₇GLGNTEVGA,
wherein
X₁ is D, S or A;
X₂ is I or V;
X₃ is V, T, M, or A;
X₄ is D, N, or T;
X₅ is L or V;
X₆ is D or E;
X₇ is A or P;
X₈ is V, I, or G;
X₉ is D, E, or S;
X₁₀ is E or D;
X₁₁ is E, L, V, or absent;
X₁₂ is A or V;
X₁₃ is A, E, or K;
X₁₄ is L, V, or A;
X₁₅ is S, L, or V;
X₁₆ is V, I, G, T, or A;
X₁₇ is G or M; and
wherein the core region comprises:
(SEQ ID NO: 10)
SGX₁SX₄X₂X₃,
wherein X₁ is three to twenty amino acids, and wherein X₂ is V or L, wherein X₃ is I or V, wherein X₄ is Y, W, F, or H, and wherein the leader and core can be separate polypeptides used in combination, a single fusion protein, or covalently linked polypeptides.

2. The substrate of claim 1, wherein the leader region comprises:
(SEQ ID NO: 2)
DLDIVX₁LDLX₂X₃DEELAAX₄SVGGLGNTEVGA,
wherein;
X₁ is D, N, or T;
X₂ is A or P;
X₃ is V, I, or G; and
X₄ is L, V, or A.

3. The substrate of claim 1, wherein the leader region comprises:
(SEQ ID NO: 3)
DLDIVDLDLAVDEELAALSVGGLGNTEVGA
(SEQ ID NO: 4)
DLDIVNLDLPIDEELAAVSVGGLGNTEVGA
(SEQ ID NO: 5)
DLDIVDLDLPIDEELAAVSIGGLGNTEVGA
(SEQ ID NO: 6)
SLDVTTVELPGEDLVEALGMGLGNTEVGA
(SEQ ID NO: 7)
SLDVMTVELPGEDLVKALGMGLGNTEVGA
(SEQ ID NO: 8)
SLDVATVELPGSDLLVEAVTMGLGNTEVGA
(SEQ ID NO: 9)
ALDVATVELPGSEVLVEAVAMGLGNTEIGA

4. The substrate of claim 1, wherein the core region comprises:
SGX1SX3X21(SEQ ID NO:11),
wherein X₁ is three to 100 amino acids and wherein the last of the three to 100 amino acids is a positively charged amino acid, and wherein X₂ is V or L, and
wherein X₃ is Y, W, F, or H.

5. The substrate of claim 1, wherein the core region comprises: SGFFX₁SWX₂₁ (SEQ ID NO:12), wherein X₁ is three to 100 amino acids, and wherein X₂ is V or L, and wherein Xa is Y, W, F, or H.

6. The substrate of claim 1, further comprising a linker region and a handle region at the C-terminus of the core region.

7. The substrate of claim 6, wherein the handle region is for amplification, detection, or purification.

8. The substrate of claim 6, wherein the handle region comprises a polypeptide or nucleic acid molecule for yeast display, phage display, mRNA display, TRAP display, or ribosome display.

9. The substrate of claim 6, wherein the linker is a flexible linker, a cleavable linker or a rigid linker.

10. A fusion protein comprising:
(a) Micromonospora dehydratase (MroB or MroC or both MroB and MroC) and an affinity tag; or
(b) Micromonospora macrocyclase (MroD) and an affinity tag.

11. The fusion protein of claim 10, wherein the affinity tag is a polyhistidine (poly-His) tag, a hemagglutinin (HA) tag, an AviTag protein C tag, a FLAG tag, a Strep-tag II, aTwin-Strep-tag, a glutathione-S-transferase (GST) tag, a C-myc tag, a chitin-binding domain, a streptavidin binding protein (SBP), a maltose binding protein (MBP), a cellulose-binding domain, a calmodulin-binding peptide, or an S-tag.

12. The fusion protein of claim 10 further comprising a linker.

13. A method of making a pyridine-based macrocyclic peptide comprising contacting the substrate for enzyme synthesis of pyridine-based macrocyclic peptides of claim 1 with MroB, MroC, and MroD.

14. The method of claim 13, wherein the MroB, MroC, and MroD are fused to an affinity tag.

15. The method of claim 13, wherein rings with 14 to 23 members are made.

16. A substrate for enzyme synthesis of pyridine-based macrocyclic peptides comprising a leader region of:
MDNVVTEAAEFADLDIDDFDLAVDEELAALSVGGLGNTEVGA (SEQ ID NO:36).
and core sequence can be SCNCFCYICCSX₁LI (SEQ ID NO:37), wherein X₁ is Y, W, F, or H, or SCX₂CX₂CX₂ICCSX₁LI (SEQ ID NO:43), wherein X₁ is Y, W, F, or H, and wherein X₂ is any amino acid; or
(SEQ ID NO: 36)
MDNVVTEAAEFADLDIDDFDLAVDEELAALSVGGLGNTEVGA,
and
a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof or a core sequence of SX₁X₃ X₁X₃ X₁X₃₁ X₁ X₁SX₂LI, (SEQ ID NO: 44) wherein X₂ is Y, W, F, or H, wherein each of X₁ are thiazole, thiazoline, oxazole, oxazoline, methyloxozole methyloxazole, methyloxazoline, or combinations thereof, and wherein X₃ is any amino acid, and wherein the leader region and core can be separate polypeptides used in combination, a single fusion protein, or covalently linked polypeptides.

17. The substrate of claim 16, further comprising a linker region and a handle region at the C-terminus of the core region.

18. A method of making pyridine-based macrocyclic peptides comprising contacting a first substrate comprising:
a leader region of
(SEQ ID NO: 36)
MDNVVTEAAEFADLDIDDFDLAVDEELAALSVGGLGNTEVGA,
and
a core sequence of SCNCFCYICCSX₁LI (SEQ ID NO:37), wherein X₁ is Y, W, F, or H, or SCX₂CX₂CX₂ICCSX₁LI (SEQ ID NO:43), wherein X₁ is Y, W, F, or H, and wherein X₂ is any amino acid,
with thiazole synthetase, TbtE, TbtF, TbtG, or TbtD such that a second substrate is formed comprising:
a leader region of
(SEQ ID NO: 36)
MDNVVTEAAEFADLDIDDFDLAVDEELAALSVGGLGNTEVGA,
and a core sequence of SX₁N X₁F X₁YI X₁ X₁SX₂LI, (SEQ ID NO:38) wherein X₂ is Y, W, F, or H, wherein each of X₁ are thiazole, thiazoline, oxazole, oxazoline, methyloxazole, methyloxazoline, or combinations thereof, or a core sequence of SX₁X₃ X₁X₃ X₁X₃₁ X₁ X₁SX₂LI, (SEQ ID NO:44) wherein X₂ is Y, W, F, or H,
wherein each of X₁ are thiazole, thiazoline, oxazole, oxazoline, methyloxazole, methyloxazoline, or combinations thereof, and wherein X₃ is any amino acid,
wherein the leader region and core sequence can be separate polypeptides used in combination, a single fusion protein, or covalently linked polypeptides; and contacting the second substrate with MroB, MroC, and MroD such that pyridine-based macrocyclic peptides are made.

19. The method of claim 18, wherein the first substrate, the second substrate, or both the first and second substrates further comprise a linker region and a handle region at the C-terminus of the core region.

20. The method of claim 18, wherein the MroB, MroC, and MroD are fused to an affinity tag.

21.-41. (canceled)
